(19) United States
(12) Patent Application Publication
(10) Pub. No.: US 2026/0183285 A1
(43) Pub. Date: Jul. 2, 2026

(54) SUBSTITUTED MACROCYCLIC AMINE MODULATORS OF OREXIN RECEPTOR 2

(71) Applicant: Vertex Pharmaceuticals Incorporated, Boston, MA (US)

(72) Inventors: Matthew Volgraf, Boston, MA (US); Liang Zhao, Boston, MA (US); Melissa LeBlanc, Boston, MA (US); Robin LaRouche-Gauthier, Boston, MA (US); Curtis Eugene Colwell, Boston, MA (US); Samir Bouayad-Gervais, Boston, MA (US); Todd J.A. Ewing, Boston, MA (US)

(73) Assignee: Vertex Pharmaceuticals Incorporated, Boston, MA (US)

(21) Appl. No.: 19/132,020

(22) PCT Filed: Nov. 22, 2023
(86) PCT No.: PCT/US2023/080929

§ 371 Date: May 22, 2025

Related U.S. Application Data
(60) Provisional application No. 63/427,672, filed on Nov. 23, 2022.
(60) Provisional application No. 63/471,848, filed on Jun. 8, 2023.

(51) Int. Cl. A61K 31/501; A61K 31/439; A61K 31/444; A61K 31/497; C07D 498/08

(52) U.S. Cl. CPC A61K 31/501; A61K 31/439; A61K 31/444; A61K 31/497; C07D 498/08

(57) ABSTRACT

Disclosed are compounds, compositions, and methods useful for preventing or treating a disease which is at least partially mediated by orexin receptor 2.

### CROSS REFERENCE TO RELATED APPLICATION

The present application claims benefit of U.S. Provisional Application No. 63/471,848, filed on Jun. 8, 2023, and U.S. Provisional Application No. 63/427,672, filed on Nov. 23, 2022, all of the contents of which are incorporated herein by reference.

### FIELD OF THE DISCLOSURE

The present disclosure generally relates to modulating (and more specifically, agonist or antagonist) compounds of orexin receptor 2 (OX2R) and methods of their use in treating conditions at least partially mediated by OX2R.

### BACKGROUND

Orexins are hypothalamic neuropeptides that are implicated in, for example, sleep/wake control and body weight homeostasis. The peptides act on two G protein-coupled receptors termed Orexin receptor type 1 (Ox1R) and Orexin receptor type 2 (Ox2R). Orexin producing neurons are exclusively localized in the lateral hypothalamic area, but their receptors are expressed in many areas of the brain.

Narcolepsy is a socially debilitating disorder characterized by an inability to properly maintain wakefulness (excessive daytime sleepiness, sleep attacks), and a pathological intrusion of signs of REM sleep into wakefulness (cataplexy, hypnagogic hallucination, sleep paralysis, etc.). Narcolepsy affects an estimated 1 in every 2,000 individuals, and is a non-progressive, life-long condition. A deficiency in orexin/Ox2R signaling causes the sleep disorder narcolepsy in humans, mice and dogs. A vast majority (>90%) of human narcoleptics lack detectable levels of orexin peptides in the cerebrospinal fluid due to a highly specific (probably autoimmune) degeneration of orexin neurons, indicating that human narcolepsy is an “orexin deficiency syndrome.”

Compounds having Ox2R agonism activity have previously been described (see, e.g., Nagahara, et al., J. Med. Chem., 2015, 58, 7931-37), however those compounds may possess some undesirable properties (e.g., poor pharmacokinetic properties and/or central nervous system penetration, poor selectivity for Ox2R, etc.) Thus, there remains a need for Ox2R agonist compounds having improved properties.

### SUMMARY

One aspect of the invention provides compounds, compositions, and methods useful for preventing or treating a disease that is at least partially mediated by orexin receptor 2. It was surprisingly discovered that the compounds disclosed and claimed herein demonstrate, among other things, high potency and selectivity for Ox2R, and improved drug-like properties, such as central nervous system penetration (e.g., as determined by a MDR1-MDCK permeability assay) and a favorable pharmacokinetic profile.

Accordingly, provided herein in some embodiments is a compound having the structure of Formula (I):

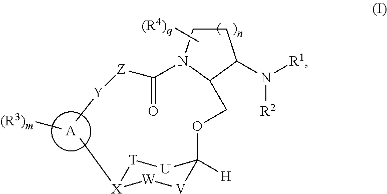

deuterated derivative, or a pharmaceutically acceptable salt or prodrug thereof;
wherein:
A is selected from the group consisting of phenyl, pyridinyl, pyridazinyl, pyrimidinyl, and pyrazinyl;
n is 0, 1, 2, or 3;
R¹ is (C₁-C₆)alkyl, (C₃-C₈)cycloalkyl, phenyl, 4- to 7-membered heterocycloalkyl, or 5- to 6-membered heteroaryl; wherein each (C₁-C₆)alkyl is optionally substituted with one or more R^{1a}, C(O)NR^{1b}R^{1c}, halo, cyano, hydroxy, or (C₁-C₆)alkoxy; and each (C₃-C₈)cycloalkyl, phenyl, 4- to 7-membered heterocycloalkyl, and 5- to 6-membered heteroaryl, is optionally substituted with one or more halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl;
R^{1a} is (C₃-C₈)cycloalkyl, phenyl, 4- to 7-membered heterocycloalkyl, or 5- to 6-membered heteroaryl, each of which is optionally substituted with one or more halo, cyano, hydroxy, (C₁-C₆)alkyl, (C₁-C₆)haloalkyl, (C₁-C₆)hydroxyalkyl, (C₁-C₆)alkoxy(C₁-C₆)alkyl, or (C₁-C₆)alkoxy;
R^{1b} and R^{1c} are independently H or (C₁-C₆)alkyl, wherein each (C₁-C₆)alkyl is optionally substituted with one or more halo, cyano, hydroxy, or (C₁-C₆)alkoxy; or R^{1a} and R^{1b} taken together with the nitrogen to which they are attached form a 3- to 7-membered heterocyclic ring;
R² is H:
R³ is halo, cyano, (C₁-C₆)alkyl, or (C₁-C₆)haloalkyl;
m is 0, 1, 2, 3, or 4;
q is 0, 1, or 2;
R⁴ is halo, (C₁-C₆)alkyl, or (C₁-C₆)haloalkyl;
X is CH;
Y is O, or absent (i.e., a bond);
Z is O, or (CR⁷R⁸)ₚ;
p is 1, 2, 3, or 4;
R⁷ and R⁸ are independently for each occurrence H, (C₁-C₃)alkyl, or (C₁-C₃)haloalkyl; or R⁷ and R⁸ taken together with the carbon to which they are attached form a 3- to 6-membered cycloalkyl;
T is CR⁹R¹⁰ or absent
U is CR¹¹R¹²;
V is CR¹³R¹⁴;
W is CR¹⁵R¹⁶ or absent, provided that only one of T or W is absent; and
R⁹, R¹⁰, R¹¹, R¹², R¹³, R¹⁴, R¹⁵, and R¹⁶ are each independently H or fluoro.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### DETAILED DESCRIPTION

### Definitions

For convenience, before further description of the present invention, certain terms employed in the specification, examples and appended claims are provided here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ³C- or ¹⁴C-enriched carbon are within the scope of this invention. As used herein, “deuterated derivative(s)” refers to a compound having the same chemical structure as a reference compound, with one or more hydrogen atoms replaced by a deuterium atom. In some embodiments, the one or more hydrogens replaced by deuterium are part of an alkyl group. In some embodiments, the one or more hydrogens replaced by deuterium are part of a methyl group. In chemical structures, deuterium may be represented as “D.”

The term “prodrug,” as used herein, encompasses compounds that, under physiological conditions, are converted into therapeutically active agents. Such a transformation can be affected, for example, by hydrolysis in blood or enzymatic transformation of the prodrug form to the parent form in blood or tissue. In other embodiments, the prodrug is converted by an enzymatic activity of the host animal.

A common method for making a prodrug is to include selected moieties that are hydrolyzed under physiological conditions to reveal the desired molecule. Prodrugs of the compounds of the invention may be, for example, amides. Amides that may be utilized as prodrugs in the present invention are phenyl amides, aliphatic (C₁-C₂₄) amides, acyloxymethyl amides, ureas, carbamates, and amino acid amides. For example, a compound of the invention that contains an NH group may be acylated at this position in its prodrug form. Other prodrug forms include esters, such as, for example phenyl esters, aliphatic (C₁-C₂₄) esters, acyloxymethyl esters, carbonates, carbamates, and amino acid esters. A thorough discussion of prodrugs is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated in its entirety herein by reference. In some embodiments, the present invention features a prodrug of any one of the formulas or compounds listed herein.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched (linear) carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted. Some examples include cyclopropyl, cyclobutyl, cyclopentyl, cyclohexyl, cycloheptyl, and cyclooctyl groups.

As used herein, the term “halocycloalkyl” refers to a cycloalkyl group as hereinbefore defined substituted with at least one halogen.

“Cycloheteroalkyl” refers to a cycloalkyl moiety, as hereinbefore defined, which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably, have 4-6 carbons and heteroatoms in the ring structure. Cycloheteroalkyl groups may be substituted or unsubstituted. Some examples include piperidinyl, piperazinyl, tetrahydrofuranyl, and tetrahydropyranyl groups.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched (linear) unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carbocyclic aryl groups include benzene, naphthalene, phenanthrene, phenol, and aniline groups, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine groups, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” or “heterocycloalkyl” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic, and may be saturated or unsaturated. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation (e.g., activating or enhancing a response) and down-regulation (e.g., inhibiting or deactivating a response).

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabeled pharmaceutical agent, for example, a radiolabeled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

One aspect of the invention relates to a compound of Formula (A):

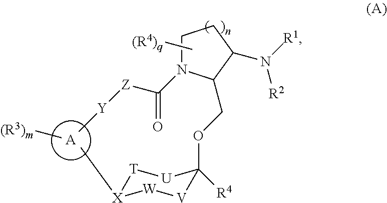

or deuterated derivative, or a pharmaceutically acceptable salt or prodrug thereof;
wherein:
A (i.e., ring A) is selected from the group consisting of phenyl, pyridinyl, pyridazinyl, pyrimidinyl, and pyrazinyl;
n is 0, 1, 2, or 3;
R¹ is (C₁-C₆)alkyl, (C₃-C₈)cycloalkyl, phenyl, 4- to 7-membered heterocycloalkyl, or 5- to 6-membered heteroaryl; wherein each (C₁-C₆)alkyl is optionally substituted with one or more R^{1a}, C(O)NR^{1b}R^{1c}, halo, cyano, hydroxy, or (C₁-C₆)alkoxy; and each (C₃-C₈)cycloalkyl, phenyl, 4- to 7-membered heterocycloalkyl, and 5- to 6-membered heteroaryl, is optionally substituted with one or more halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl;
R^{1a} is (C₃-C₈)cycloalkyl, phenyl, 4- to 7-membered heterocycloalkyl, or 5- to 6-membered heteroaryl, each of which is optionally substituted with one or more halo, cyano, hydroxy, (C₁-C₆)alkyl, (C₁-C₆)haloalkyl, (C₁-C₆)hydroxyalkyl, (C₁-C₆)alkoxy(C₁-C₆)alkyl, or (C₁-C₆)alkoxy;
R^{1b} and R^{1c} are independently H or (C₁-C₆)alkyl, wherein each (C₁-C₆)alkyl is optionally substituted with one or more halo, cyano, hydroxy, or (C₁-C₆)alkoxy; or R^{1a} and R^{1b} taken together with the nitrogen to which they are attached form a 3- to 7-membered heterocyclic ring;
R² is H:
R³ is halo, cyano, (C₁-C₆)alkyl, or (C₁-C₆)haloalkyl;
m is 0, 1, 2, 3, or 4;
q is 0, 1, or 2;
R⁴ is, in each instance, independently selected from hydrogen atom (H), halo, (C₁-C₆)alkyl, and (C₁-C₆)haloalkyl;
X is CH;
Y is O, or absent (i.e., a bond);
Z is O, or (CR⁷R⁸)ₚ;
p is 1, 2, 3, or 4;
R⁷ and R⁸ are independently for each occurrence H, (C₁-C₃)alkyl, or (C₁-C₃)haloalkyl; or
R⁷ and R⁸ taken together with the carbon to which they are attached form a 3- to 6-membered cycloalkyl;
T is CR⁹R¹⁰ or absent
U is CR¹¹R¹²;
V is CR¹³R¹⁴;
W is CR¹⁵R¹⁶ or absent, provided that only one of T or W is absent;
and
R⁹, R¹⁰, R¹¹, R¹², R¹³, R¹⁴, R¹⁵, and R¹⁶ are each independently H or fluoro.

One aspect of the invention relates to a compound of Formula (I):

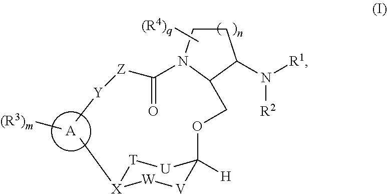

or deuterated derivative, or a pharmaceutically acceptable salt or prodrug thereof;
wherein:
A is selected from the group consisting of phenyl, pyridinyl, pyridazinyl, pyrimidinyl, and pyrazinyl;
n is 0, 1, 2, or 3;
R¹ is (C₁-C₆)alkyl, (C₃-C₈)cycloalkyl, phenyl, 4- to 7-membered heterocycloalkyl, or 5- to 6-membered heteroaryl; wherein each (C₁-C₆)alkyl is optionally substituted with one or more R^{1a}, C(O)NR^{1b}R^{1c}, halo, cyano, hydroxy, or (C₁-C₆)alkoxy; and each (C₃-C₈)cycloalkyl, phenyl, 4- to 7-membered heterocycloalkyl, and 5- to 6-membered heteroaryl, is optionally substituted with one or more halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl;
R^{1a} is (C₃-C₈)cycloalkyl, phenyl, 4- to 7-membered heterocycloalkyl, or 5- to 6-membered heteroaryl, each of which is optionally substituted with one or more halo, cyano, hydroxy, (C₁-C₆)alkyl, (C₁-C₆)haloalkyl, (C₁-C₆)hydroxyalkyl, (C₁-C₆)alkoxy(C₁-C₆)alkyl, or (C₁-C₆)alkoxy;
R^{1b} and R^{1c} are independently H or (C₁-C₆)alkyl, wherein each (C₁-C₆)alkyl is optionally substituted with one or more halo, cyano, hydroxy, or (C₁-C₆)alkoxy; or R^{1a} and R^{1b} taken together with the nitrogen to which they are attached form a 3- to 7-membered heterocyclic ring;
R² is H:
R³ is halo, cyano, (C₁-C₆)alkyl, or (C₁-C₆)haloalkyl;
m is 0, 1, 2, 3, or 4;
q is 0, 1, or 2;
R⁴ is halo, (C₁-C₆)alkyl, or (C₁-C₆)haloalkyl;
X is CH;
Y is O, or absent (i.e., a bond);
Z is O, or (CR⁷R⁸)ₚ;
p is 1, 2, 3, or 4;
R⁷ and R⁸ are independently for each occurrence H. (C₁-C₃)alkyl, or (C₁-C₃)haloalkyl; or R⁷ and R⁸ taken together with the carbon to which they are attached form a 3- to 6-membered cycloalkyl;
T is CR⁹R¹⁰ or absent
U is CR¹¹R¹²;
V is CR¹³R¹⁴;
W is CR¹⁵R¹⁶ or absent, provided that only one of T or W is absent; and
R⁹, R¹⁰, R¹¹, R¹², R¹³, R¹⁴, R¹⁵, and R¹⁶ are each independently H or fluoro. For clarity, in Formula (I) and other formulae described herein, the presence of the capital letter h (“H”) in a structure is intended to refer to a hydrogen atom.

As X is CH, Formula (I) may also be represented as follows:

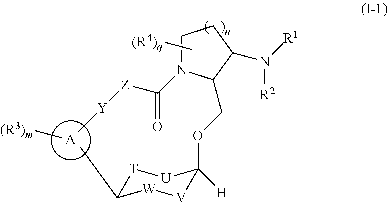

or deuterated derivative, or a pharmaceutically acceptable salt or prodrug thereof.

The compounds of Formulae (A), (I) or (I-1) may also be an enriched or isolated chiral form (e.g., enantiomeric or diastereomeric form) or a racemic mixture of two or more chiral forms.

In some embodiments, the compound of Formula A is an enriched or isolated chiral form having the structure of Formula Ia:

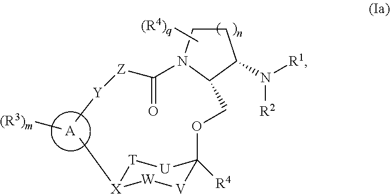

or deuterated derivative, or a pharmaceutically acceptable salt or prodrug thereof, while in other embodiments, the invention is a compound of Formula Ib:

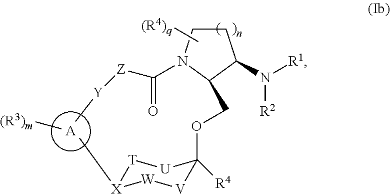

or deuterated derivative, or a pharmaceutically acceptable salt or prodrug thereof. Racemic mixtures of the above are also contemplated.

In some embodiments, the compound of Formula I is an enriched or isolated chiral form having the structure of Formula Ia-1:

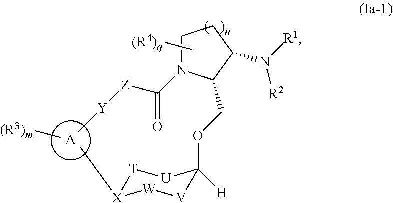

or deuterated derivative, or a pharmaceutically acceptable salt or prodrug thereof, while in other embodiments, the invention is a compound of Formula Ib-1:

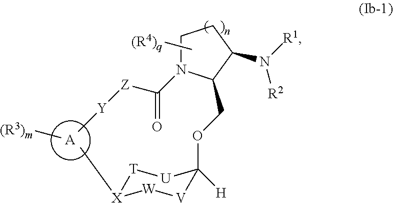

or deuterated derivative, or a pharmaceutically acceptable salt or prodrug thereof. Racemic mixtures of the above are also contemplated.

In particular embodiments of Formula (I), the compounds of the invention are of Formula I-2:

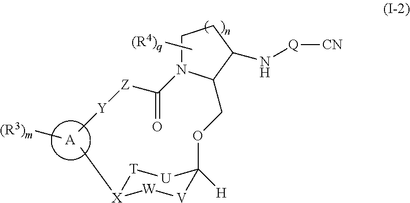

or deuterated derivative, or a pharmaceutically acceptable salt or prodrug thereof;
wherein Q is selected from (C₁-C₆)alkyl, (C₃-C₈)cycloalkyl, phenyl, 4- to 7-membered heterocycloalkyl, and 5- to 6-membered heteroaryl, as provided above for R¹, including any of the examples and optional substitutions provided above for R¹. In some embodiments, Q is (C₁-C₆)alkyl substituted with a (C₃-C₈)cycloalkyl. In the above formula, R² is taken as H. The group CN is a nitrile group.

As X is CH, Formula (I-2) can also be represented as follows:

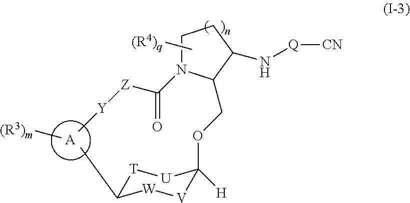

or deuterated derivative, or a pharmaceutically acceptable salt or prodrug thereof.

Chiral forms of Formulas (I-2) and (I-3) are also considered herein, such as represented by the following formulas:

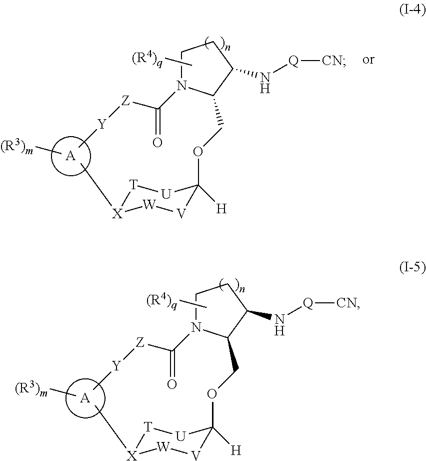

or deuterated derivatives or a pharmaceutically acceptable salts or prodrugs thereof. Racemic mixtures of the above are also contemplated.

Since X is CH, Formulas (I-4) and (I-5) can also be represented as follows:

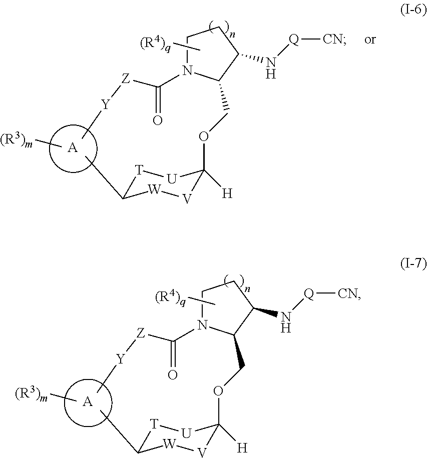

or deuterated derivatives, or pharmaceutically acceptable salts or prodrugs thereof.

In some embodiments, for any of the above formulas, n is 1, while in other embodiments, n is 2.

In some embodiments, for any of the above formulas, A (i.e., ring A) is phenyl.

In some embodiments, for any of the above formulas, m is 0, while in other embodiments, m is 1, and in still other embodiments, m is 2.

In some embodiments, for any of the above formulas, R³ is halo. In certain preferred embodiments, R³ is fluoro.

In some embodiments, for any of the above formulas, A is

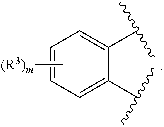

In some embodiments, for any of the above formulas, A is,

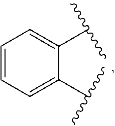 [SMILES: *c1ccccc1*]

while in other embodiments, A is

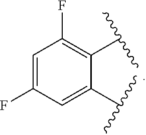 [SMILES: *c1cc(F)cc(F)c1*]

In some embodiments, for any of the above formulas, R¹ is (C₁-C₆)alkyl optionally substituted with one or more fluoro, cyano, hydroxy, R^{1a}, or C(O)NR^{1b}R^{1c}. In some embodiments, R¹ is (C₁-C₆)alkyl substituted with at least one fluoro or cyano. In some embodiments, R¹ is (C₁-C₆)alkyl substituted with cyano. In other embodiments, R¹ is (C₁-C₆)alkyl substituted with a (C₃-C₈)cycloalkyl group.

In some embodiments, for any of the above formulas, R¹ is methyl optionally substituted with one or more fluoro, cyano, hydroxy, R^{1a}, or C(O)NR^{1b}R^{1c}.

In some embodiments, for any of the above formulas, R^{1a} is (C₃-C₈)cycloalkyl, phenyl, 4- to 5-membered heterocycloalkyl, or 5- to 6-membered heteroaryl, each of which is optionally substituted with one or more of fluoro, cyano, (C₁-C₃)alkyl, (C₁-C₃)fluoroalkyl, or (C₁-C₃)alkoxy. In more particular embodiments, R^{1a} is cyclopropyl, phenyl, 4- to 5-membered heterocycloalkyl, or 5- to 6-membered heteroaryl, each of which is optionally substituted with one or more of fluoro, cyano, cyclopropyl, methyl, or trifluoromethyl. In some embodiments, R^{1a} is cyclopropyl, cyclobutyl, phenyl, oxetanyl, tertrahydrofuranyl, or tetrahydropyranyl, each of which is optionally substituted with one or more of fluoro, cyano, cyclopropyl, methyl, or trifluoromethyl.

In some embodiments, for any of the above formulas, R¹ is (C₁-C₆)alkyl substituted with C(O)NR^{1b}R^{1c}. In particular embodiments, R^{1b} and R^{1c} are each H.

In particular embodiments, R is:

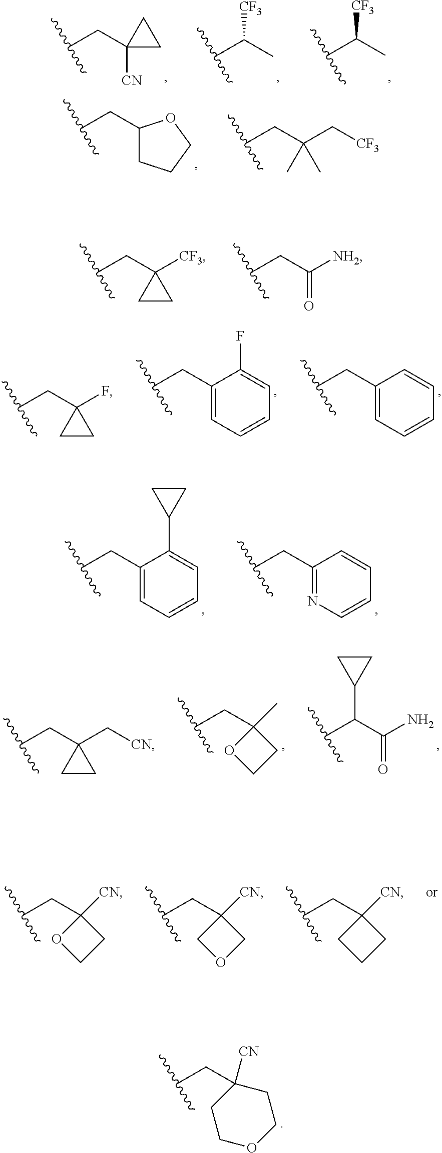

In further particular embodiments, R¹ is:

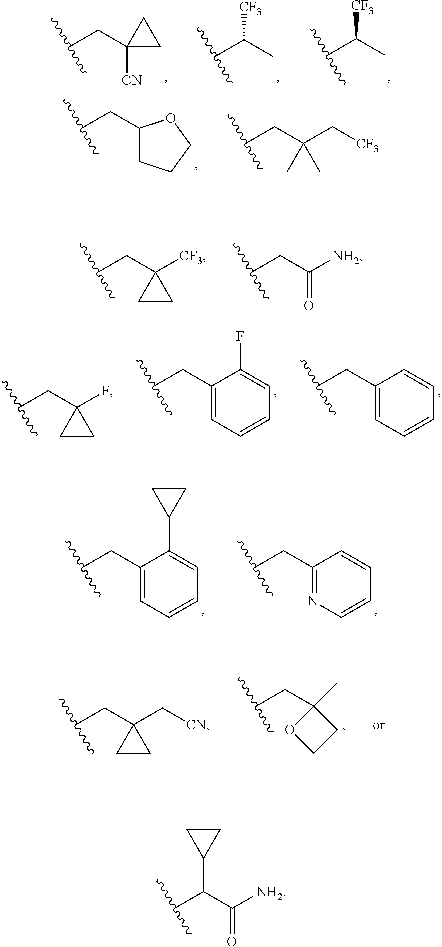

In other embodiments, R¹ is phenyl, 2-pyridyl, 3-pyridyl, 4-pyridyl, pyridazinyl, pyrazinyl, or diazoyl, each of which is optionally substituted with one or more of halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl.

In some embodiments, R¹ is phenyl, 2-pyridyl, 3-pyridyl, 4-pyridyl, pyridazinyl, pyrazinyl, or diazoyl, each of which is optionally substituted with one or more of fluoro, cyano, hydroxy, (C₁-C₃)alkyl, (C₁-C₃)fluoroalkyl, or (C₁-C₃)alkoxy. In more particular embodiments, R¹ is phenyl, 2-pyridyl, 3-pyridyl, 4-pyridyl, or diazoyl, each of which is optionally substituted with one or more of fluoro, cyano, hydroxy, (C₁-C₃)alkyl, (C₁-C₃)fluoroalkyl, (C₁-C₃)alkoxy or 4- to 7-membered heterocycloalkyl. In still more particular embodiments, R¹ is phenyl, 2-pyridyl, 3-pyridyl, 4-pyridyl, or diazoyl, each of which is optionally substituted with one or more of fluoro, cyano, methyl, trifluoromethyl, methoxy or oxetane.

In some embodiments, R¹ is:

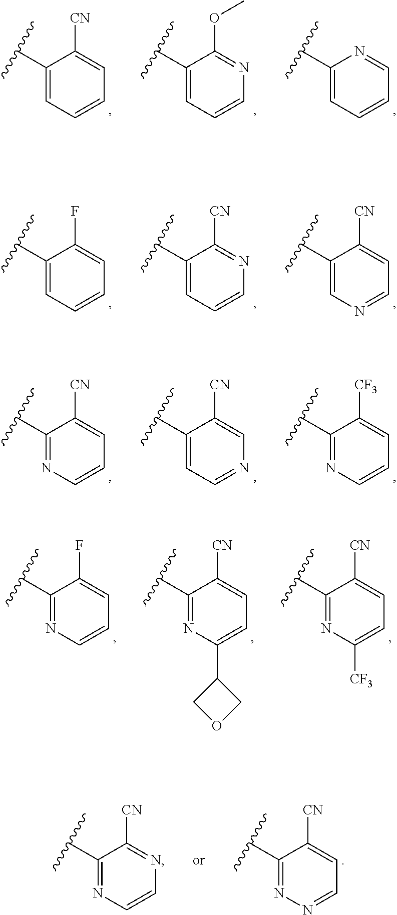

In some embodiments, R¹ is:

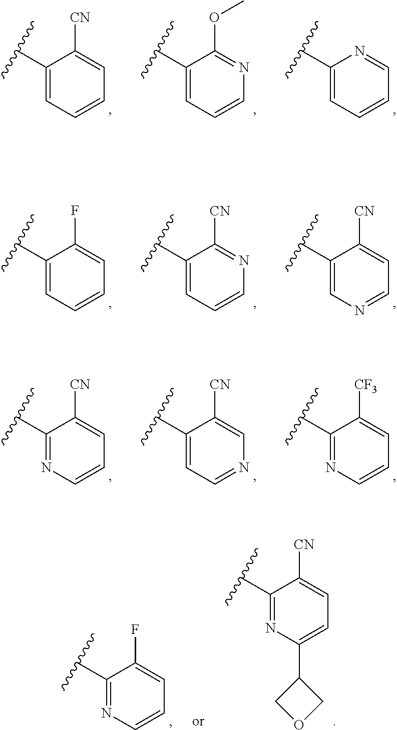

In other embodiments, R^{f} is (C₃-C₅)cycloalkyl or 4- to 6-membered heterocycloalkyl, each of which is optionally substituted with one or more of halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl.

In some embodiments, R^{f} is:

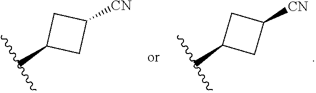 [SMILES: *[C@H]1C[C@H](C#N)C1 ; *[C@H]1C[C@@H](C#N)C1]

In other embodiments, R¹ is:

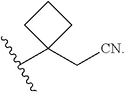 [SMILES: *C1(CC#N)CCC1]

In still other embodiments, R¹ is:

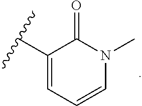 [SMILES: *c1cccn(C)c1=O]

In some embodiments, R¹ is substituted with at least one fluoro or cyano. In other embodiments, R¹ is substituted with cyano.

In some embodiments, for any of the above formulas, Y is O. In other embodiments, Y is absent (i.e., a bond).

In some embodiments, for any of the above formulas, R⁷ and R⁸ are each CH₃; or R⁷ and R⁸ taken together with the atom to which they are attached form a cyclopropyl. In other embodiments, R⁷ and R⁸ are each H.

In some embodiments, for any of the above formulas, p is 1.

In some embodiments, for any of the above formulas, R⁹, R¹⁰, R¹¹, R¹², R¹³, R¹⁴, R¹⁵, and R¹⁶ are each H.

In some embodiments, for any of the above formulas in which two instances of R⁴ are present, the R⁴ groups are both hydrogen atom (H).

In some embodiments, the compound is a compound of Table 1A, or a deuterated derivative, or a pharmaceutically acceptable salt or prodrug thereof. In other embodiments, the compound is a compound of Table 1A, or a pharmaceutically acceptable salt thereof. In certain particular embodiments, the compound is a compound of Table 1A.

TABLE 1A

| Cmpd # | Structure | IUPAC |
|---|---|---|
| 1 | 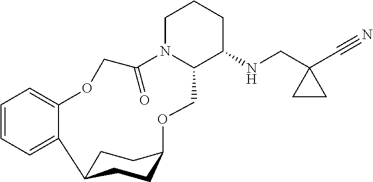 | 1-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)cyclopropane-1-carbonitrile |
| 2 | 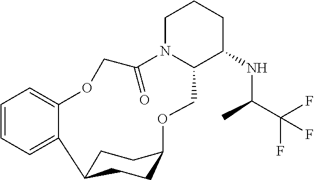 [SMILES: C[C@@H](N[C@H]1CCCN2C(=O)COc3ccccc3[C@H]3CC[C@H](CC3)OC[C@@H]12)C(F)(F)F] | (2¹S,2⁴S,5²R,5³S)-5³-(((R)-1,1,1-trifluoropropan-2- yl)amino)-3,8-dioxa-5(2,1)-piperidina-1(1,2)-benzena- 2(1,4)-cyclohexanacyclooctaphan-6-one |
| 3 | 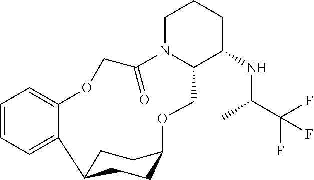 [SMILES: C[C@H](N[C@H]1CCCN2C(=O)COc3ccccc3[C@H]3CC[C@H](CC3)OC[C@@H]12)C(F)(F)F] | (2¹S,2⁴S,5²R,5³S)-5³-(((S)-1,1,1-trifluoropropan-2- yl)amino)-3,8-dioxa-5(2,1)-piperidina-1(1,2)-benzena- 2(1,4)-cyclohexanacyclooctaphan-6-one |
| 4 | 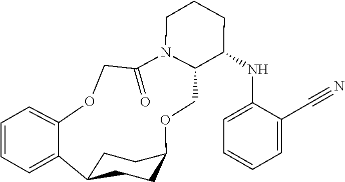 [SMILES: N#Cc1ccccc1N[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)benzonitrile |
| 5 | 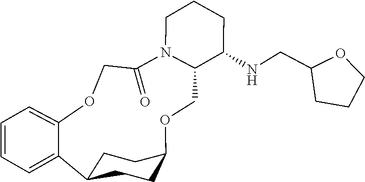 [SMILES: O=C1COc2ccccc2[C@H]2CC[C@H](CC2)OC[C@H]2[C@@H](NCC3CCCO3)CCCN12] | (2¹S,2⁴S,5²R,5³S)-5³-(((tetrahydrofuran-2- yl)methyl)amino)-3,8-dioxa-5(2,1)-piperidina-1(1,2)- benzena-2(1,4)-cyclohexanacyclooctaphan-6-one |
| 6 | 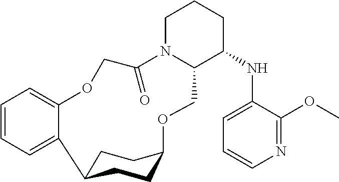 | (2¹S,2⁴S,5²R,5³S)-5³-((2-methoxypyridin-3-yl)amino)- 3,8-dioxa-5(2,1)-piperidina-1(1,2)-benzena-2(1,4)- cyclohexanacyclooctaphan-6-one |
| 7 | 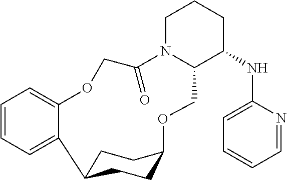 [SMILES: O=C1COc2ccccc2[C@H]2CC[C@H](CC2)OC[C@H]2[C@@H](Nc3ccccn3)CCCN12] | (2¹S,2⁴S,5²R,5³S)-5³-(pyridin-2-ylamino)-3,8-dioxa- 5(2,1)-piperidina-1(1,2)-benzena-2(1,4)- cyclohexanacyclooctaphan-6-one |
| 8 | 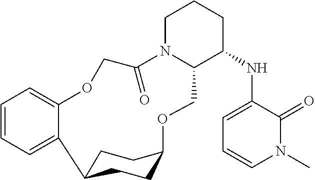 [SMILES: Cn1cccc(N[C@H]2CCCN3C(=O)COc4ccccc4[C@H]4CC[C@H](CC4)OC[C@@H]23)c1=O] | (2¹S,2⁴S,5²R,5³S)-5³-((1-methyl-2-oxo-1,2- dihydropyridin-3-yl)amino)-3,8-dioxa-5(2,1)- piperidina-1(1,2)-benzena-2(1,4)- cyclohexanacyclooctaphan-6-one |
| 9 | 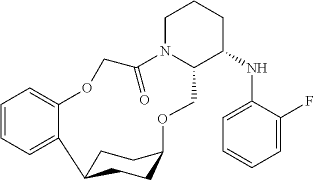 [SMILES: O=C1COc2ccccc2[C@H]2CC[C@H](CC2)OC[C@H]2[C@@H](Nc3ccccc3F)CCCN12] | (2¹S,2⁴S,5²R,5³S)-5³-((2-fluorophenyl)amino)-3,8- dioxa-5(2,1)-piperidina-1(1,2)-benzena-2(1,4)- cyclohexanacyclooctaphan-6-one |
| 10 | 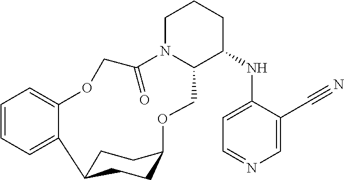 [SMILES: N#Cc1cnccc1N[C@H]1CCCN2C(=O)COc3ccccc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)picolinonitrile |
| 11 | 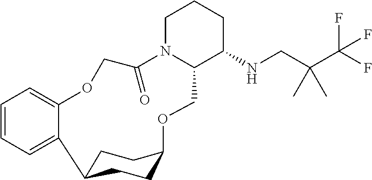 [SMILES: CC(C)(CN[C@H]1CCCN2C(=O)COc3ccccc3[C@H]3CC[C@H](CC3)OC[C@@H]12)C(F)(F)F] | (2¹S,2⁴S,5²R,5³S)-5³-((3,3,3-trifluoro-2,2- dimethylpropyl)amino)-3,8-dioxa-5(2,1)-piperidina- 1(1,2)-benzena-2(1,4)-cyclohexanacyclooctaphan-6-one |
| 12 | 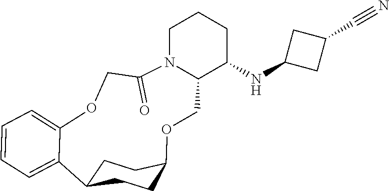 [SMILES: N#C[C@H]1C[C@H](N[C@H]2CCCN3C(=O)COc4ccccc4[C@H]4CC[C@H](CC4)OC[C@@H]23)C1] | (1S,3r)-3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)cyclobutane-1- carbonitrile |
| 13 | 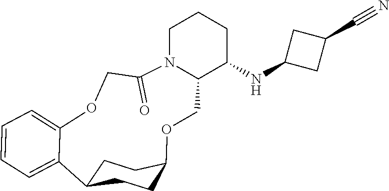 [SMILES: N#C[C@H]1C[C@@H](N[C@H]2CCCN3C(=O)COc4ccccc4[C@H]4CC[C@H](CC4)OC[C@@H]23)C1] | (1R,3s)-3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)cyclobutane-1- carbonitrile |
| 14 | 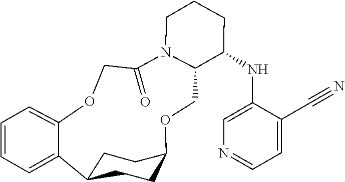 [SMILES: N#Cc1ccncc1N[C@H]1CCCN2C(=O)COc3ccccc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)isonicotinonitrile |
| 15 | 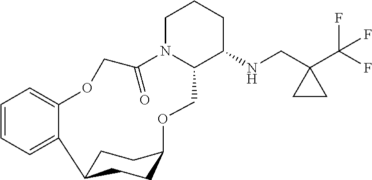 [SMILES: O=C1COc2ccccc2[C@H]2CC[C@H](CC2)OC[C@H]2[C@@H](NCC3(C(F)(F)F)CC3)CCCN12] | (2¹S,2⁴S,5²R,5³S)-5³-(((1- (trifluoromethyl)cyclopropyl)methyl)amino)-3,8-dioxa- 5(2,1)-piperidina-1(1,2)-benzena-2(1,4)- cyclohexanacyclooctaphan-6-one |
| 16 | 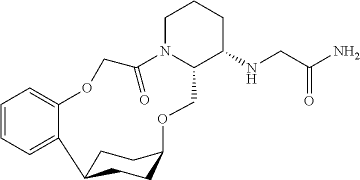 [SMILES: NC(=O)CN[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)acetamide |
| 17 | 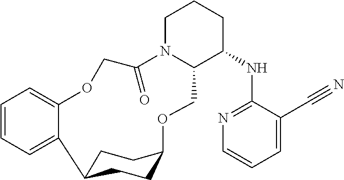 [SMILES: N#Cc1cccnc1N[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)nicotinonitrile |
| 18 | 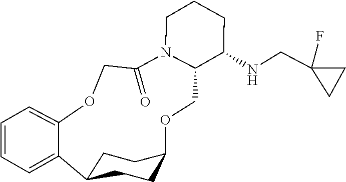 [SMILES: O=C1COc2ccccc2[C@H]2CC[C@H](CC2)OC[C@H]2[C@@H](NCC3(F)CC3)CCCN12] | (2¹S,2⁴S,5²R,5³S)-5³-(((1- fluorocyclopropyl)methyl)amino)-3,8-dioxa-5(2,1)- piperidina-1(1,2)-benzena-2(1,4)- cyclohexanacyclooctaphan-6-one |
| 19 | 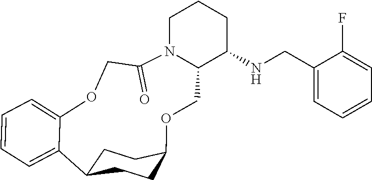 [SMILES: O=C1COc2ccccc2[C@H]2CC[C@H](CC2)OC[C@H]2[C@@H](NCc3ccccc3F)CCCN12] | (2¹S,2⁴S,5²R,5³S)-5³-((2-fluorobenzyl)amino)-3,8- dioxa-5(2,1)-piperidina-1(1,2)-benzena-2(1,4)- cyclohexanacyclooctaphan-6-one |
| 20 | 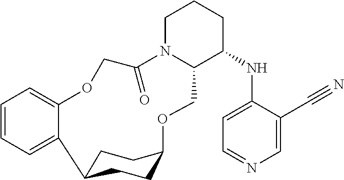 [SMILES: N#Cc1cnccc1N[C@H]1CCCN2C(=O)COc3ccccc3[C@H]3CC[C@H](CC3)OC[C@@H]12] | 4-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)nicotinonitrile |
| 21 | 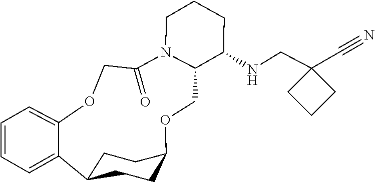 [SMILES: N#CC1(CN[C@H]2CCCN3C(=O)COc4ccccc4[C@H]4CC[C@H](CC4)OC[C@@H]23)CCC1] | 1-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)cyclobutane-1-carbonitrile |
| 22 | 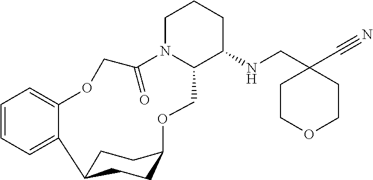 [SMILES: N#CC1(CN[C@H]2CCCN3C(=O)COc4ccccc4[C@H]4CC[C@H](CC4)OC[C@@H]23)CCOCC1] | 4-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)tetrahydro-2H-pyran-4-carbonitrile |
| 23 | 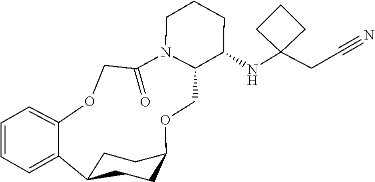 [SMILES: N#CCC1(N[C@H]2CCCN3C(=O)COc4ccccc4[C@H]4CC[C@H](CC4)OC[C@@H]23)CCC1] | 2-(1-(((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)cyclobutyl)acetonitrile |
| 24 | 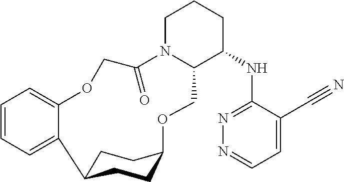 | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)pyridazine-4- carbonitrile |
| 25 | 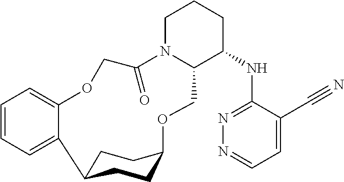 | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)pyrazine-2- carbonitrile |
| 26 | 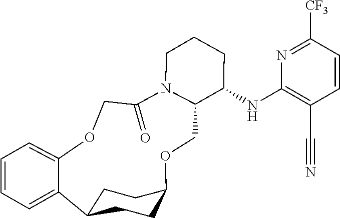 [SMILES: N#Cc1ccc(C(F)(F)F)nc1N[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)-6- (trifluoromethyl)nicotinonitrile |
| 27 | 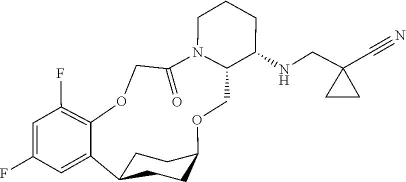 [SMILES: N#CC1(CN[C@H]2CCCN3C(=O)COc4c(F)cc(F)cc4[C@H]4CC[C@H](CC4)OC[C@@H]23)CC1] | 1-((((2¹S,2⁴S,5²R,5³S)-13,15-difluoro-6-oxo-3,8-dioxa- 5(2,1)-piperidina-1(1,2)-benzena-2(1,4)- cyclohexanacyclooctaphane-5³- yl)amino)methyl)cyclopropane-1-carbonitrile |
| 28 | 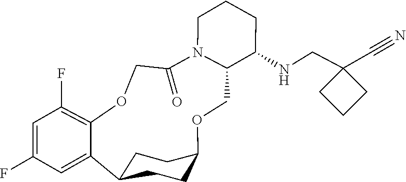 [SMILES: N#CC1(CN[C@H]2CCCN3C(=O)COc4c(F)cc(F)cc4[C@H]4CC[C@H](CC4)OC[C@@H]23)CCC1] | 1-((((2¹S,2⁴S,5²R,5³S)-13,15-difluoro-6-oxo-3,8-dioxa- 5(2,1)-piperidina-1(1,2)-benzena-2(1,4)- cyclohexanacyclooctaphane-5³- yl)amino)methyl)cyclobutane-1-carbonitrile |
| 29 | 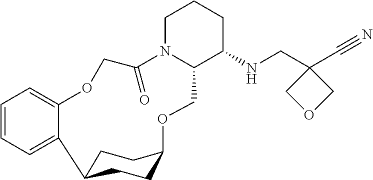 [SMILES: N#CC1(CN[C@H]2CCCN3C(=O)COc4ccccc4[C@H]4CC[C@H](CC4)OC[C@@H]23)COC1] | 3-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)oxetane-3-carbonitrile |
| 30 | 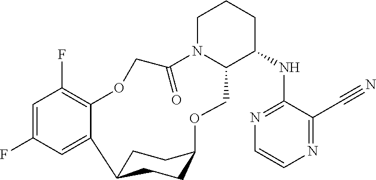 [SMILES: N#Cc1nccnc1N[C@H]1CCCN2C(=O)COc3c(F)cc(F)cc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)pyrazine-2- carbonitrile |
| 31 | 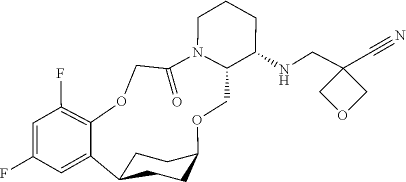 [SMILES: N#CC1(CN[C@H]2CCCN3C(=O)COc4c(F)cc(F)cc4[C@H]4CC[C@H](CC4)OC[C@@H]23)COC1] | 3-((((2¹S,2⁴S,5²R,5³S)-13,15-difluoro-6-oxo-3,8-dioxa- 5(2,1)-piperidina-1(1,2)-benzena-2(1,4)- cyclohexanacyclooctaphane-5³- yl)amino)methyl)oxetane-3-carbonitrile |
| 32 | 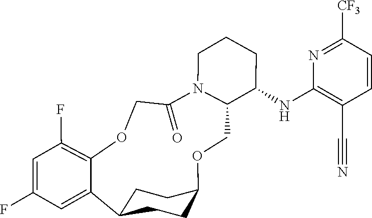 [SMILES: N#Cc1ccc(C(F)(F)F)nc1N[C@H]1CCCN2C(=O)COc3c(F)cc(F)cc3[C@H]3CC[C@H](CC3)OC[C@@H]12] | 2-(((2¹S,2⁴S,5²R,5³S)-13,15-difluoro-6-oxo-3,8-dioxa- 5(2,1)-piperidina-1(1,2)-benzena-2(1,4)- cyclohexanacyclooctaphane-5³-yl)amino)-6- (trifluoromethyl)nicotinonitrile |
| 33 | 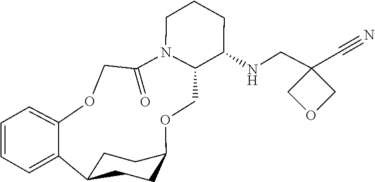 [SMILES: N#CC1(CN[C@H]2CCCN3C(=O)COc4ccccc4[C@H]4CC[C@H](CC4)OC[C@@H]23)COC1] | 2-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)oxetane-2-carbonitrile |

In some embodiments, the compound is a compound of Table 1B, or a deuterated derivative, or a pharmaceutically acceptable salt or prodrug thereof. In other embodiments, the compound is a compound of Table 1B, or a pharmaceutically acceptable salt thereof. In other embodiments, the compound is a compound of Table 1B.

TABLE 1B

| Cmpd # | Structure | IUPAC |
|---|---|---|
| 1 | 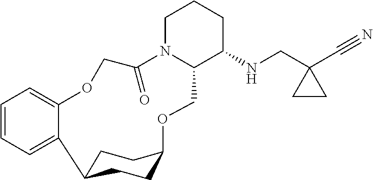 [SMILES: N#CC1(CN[C@H]2CCCN3C(=O)COc4ccccc4[C@H]4CC[C@H](CC4)OC[C@@H]23)CC1] | 1-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)cyclopropane-1-carbonitrile |
| 2 | 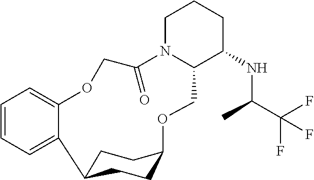 [SMILES: C[C@@H](N[C@H]1CCCN2C(=O)COc3ccccc3[C@H]3CC[C@H](CC3)OC[C@@H]12)C(F)(F)F] | (2¹S,2⁴S,5²R,5³S)-5³-(((R)-1,1,1-trifluoropropan-2- yl)amino)-3,8-dioxa-5(2,1)-piperidina-1(1,2)-benzena- 2(1,4)-cyclohexanacyclooctaphan-6-one |
| 3 | 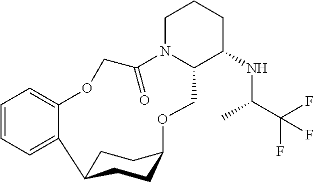 | (2¹S,2⁴S,5²R,5³S)-5³-(((S)-1,1,1-trifluoropropan-2- yl)amino)-3,8-dioxa-5(2,1)-piperidina-1(1,2)-benzena- 2(1,4)-cyclohexanacyclooctaphan-6-one |
| 4 | 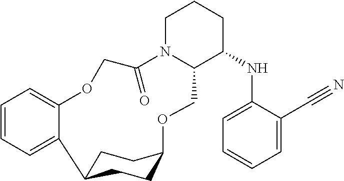 [SMILES: N#Cc1ccccc1N[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)benzonitrile |
| 5 | 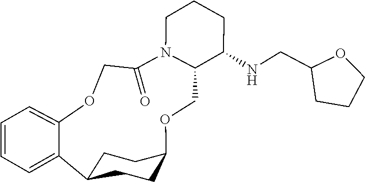 [SMILES: O=C1COc2ccccc2[C@H]2CC[C@H](CC2)OC[C@H]2[C@@H](NCC3CCCO3)CCCN12] | (2¹S,2⁴S,5²R,5³S)-5³-(((tetrahydrofuran-2- yl)methyl)amino)-3,8-dioxa-5(2,1)-piperidina-1(1,2)- benzena-2(1,4)-cyclohexanacyclooctaphan-6-one |
| 6 | 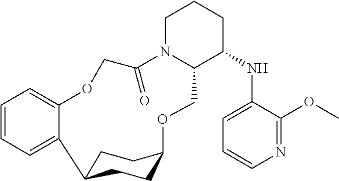 [SMILES: COc1ncccc1N[C@H]1CCCN2C(=O)COc3ccccc3[C@H]3CC[C@H](CC3)OC[C@@H]12] | (2¹S,2⁴S,5²R,5³S)-5³-((2-methoxypyridin-3-yl)amino)- 3,8-dioxa-5(2,1)-piperidina-1(1,2)-benzena-2(1,4)- cyclohexanacyclooctaphan-6-one |
| 7 | 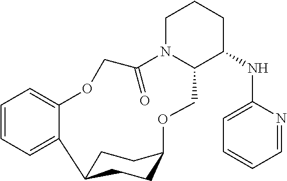 [SMILES: O=C1COc2ccccc2[C@H]2CC[C@H](CC2)OC[C@H]2[C@@H](Nc3ccccn3)CCCN12] | (2¹S,2⁴S,5²R,5³S)-5³-(pyridin-2-ylamino)-3,8-dioxa- 5(2,1)-piperidina-1(1,2)-benzena-2(1,4)- cyclohexanacyclooctaphan-6-one |
| 8 | 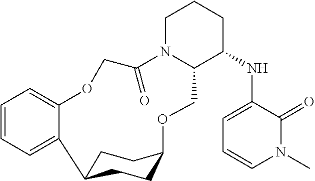 [SMILES: Cn1cccc(N[C@H]2CCCN3C(=O)COc4ccccc4[C@H]4CC[C@H](CC4)OC[C@@H]23)c1=O] | (2¹S,2⁴S,5²R,5³S)-5³-((1-methyl-2-oxo-1,2- dihydropyridin-3-yl)amino)-3,8-dioxa-5(2,1)-piperidina- 1(1,2)-benzena-2(1,4)-cyclohexanacyclooctaphan-6-one |
| 9 | 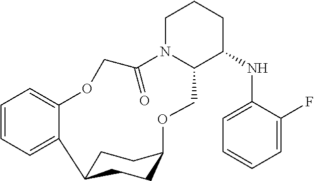 [SMILES: O=C1COc2ccccc2[C@H]2CC[C@H](CC2)OC[C@H]2[C@@H](Nc3ccccc3F)CCCN12] | (2¹S,2⁴S,5²R,5³S)-5³-((2-fluorophenyl)amino)-3,8- dioxa-5(2,1)-piperidina-1(1,2)-benzena-2(1,4)- cyclohexanacyclooctaphan-6-one |
| 10 | 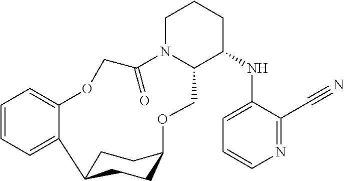 [SMILES: N#Cc1ncccc1N[C@H]1CCCN2C(=O)COc3ccccc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)picolinonitrile |
| 11 | 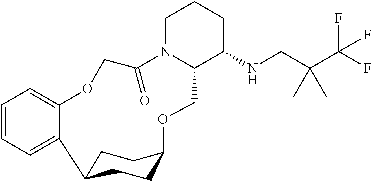 [SMILES: CC(C)(CN[C@H]1CCCN2C(=O)COc3ccccc3[C@H]3CC[C@H](CC3)OC[C@@H]12)C(F)(F)F] | (2¹S,2⁴S,5²R,5³S)-5³-((3,3,3-trifluoro-2,2- dimethylpropyl)amino)-3,8-dioxa-5(2,1)-piperidina- 1(1,2)-benzena-2(1,4)-cyclohexanacyclooctaphan-6-one |
| 12 | 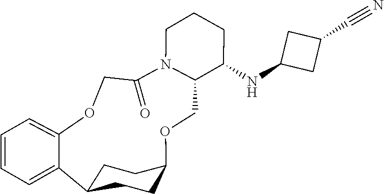 [SMILES: N#C[C@H]1C[C@H](N[C@H]2CCCN3C(=O)COc4ccccc4[C@H]4CC[C@H](CC4)OC[C@@H]23)C1] | (1S,3r)-3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)cyclobutane-1- carbonitrile |
| 13 | 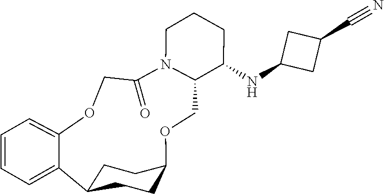 [SMILES: N#C[C@H]1C[C@@H](N[C@H]2CCCN3C(=O)COc4ccccc4[C@H]4CC[C@H](CC4)OC[C@@H]23)C1] | (1R,3s)-3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)cyclobutane-1- carbonitrile |
| 14 | 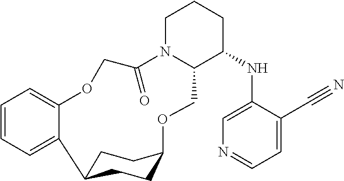 [SMILES: N#Cc1ccncc1N[C@H]1CCCN2C(=O)COc3ccccc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)isonicotinonitrile |
| 15 | 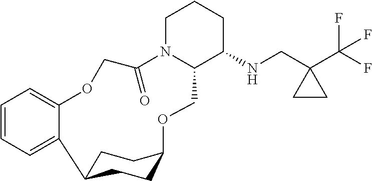 [SMILES: O=C1COc2ccccc2[C@H]2CC[C@H](CC2)OC[C@H]2[C@@H](NCC3(C(F)(F)F)CC3)CCCN12] | (2¹S,2⁴S,5²R,5³S)-5³-(((1- (trifluoromethyl)cyclopropyl)methyl)amino)-3,8-dioxa- 5(2,1)-piperidina-1(1,2)-benzena-2(1,4)- cyclohexanacyclooctaphan-6-one |
| 16 | 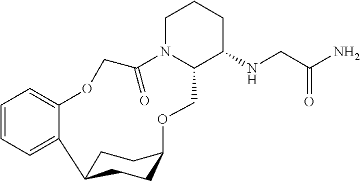 [SMILES: NC(=O)CN[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)acetamide |
| 17 | 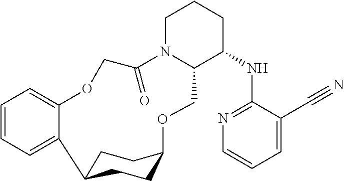 [SMILES: N#Cc1cccnc1N[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)nicotinonitrile |
| 18 | 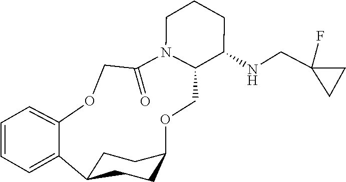 [SMILES: O=C1COc2ccccc2[C@H]2CC[C@H](CC2)OC[C@H]2[C@@H](NCC3(F)CC3)CCCN12] | (2¹S,2⁴S,5²R,5³S)-5³-(((1- fluorocyclopropyl)methyl)amino)-3,8-dioxa-5(2,1)- piperidina-1(1,2)-benzena-2(1,4)- cyclohexanacyclooctaphan-6-one |
| 19 | 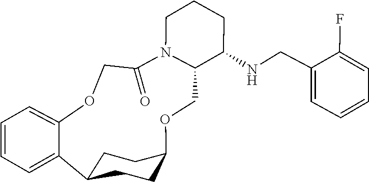 [SMILES: O=C1COc2ccccc2[C@H]2CC[C@H](CC2)OC[C@H]2[C@@H](NCc3ccccc3F)CCCN12] | (2¹S,2⁴S,5²R,5³S)-5³-((2-fluorobenzyl)amino)-3,8- dioxa-5(2,1)-piperidina-1(1,2)-benzena-2(1,4)- cyclohexanacyclooctaphan-6-one |
| 20 | 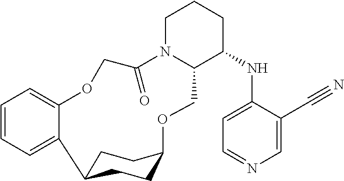 [SMILES: N#Cc1cnccc1N[C@H]1CCCN2C(=O)COc3ccccc3[C@H]3CC[C@H](CC3)OC[C@@H]12] | 4-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)nicotinonitrile |

In certain embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O—(C₁-C₄)alkyl can be suitably deuterated (e.g., —CD₃, —OCD₃).

Any compound of the invention can also be radiolabeled for the preparation of a radiopharmaceutical agent.

For the avoidance of doubt, this disclosure is directed to compounds disclosed herein (e.g., Formulas A, I, I-1, Ia, Ib, Ia-1, Ib-1, I-2, I-3, I-4, I-5, I-6, and I-7) and the compounds described in Tables 1A and 1B), deuterated derivatives of those compounds, and pharmaceutically acceptable salts or prodrugs thereof. In some embodiments, the disclosure is directed to compounds disclosed herein (e.g., Formulas A, I, I-1, Ia, Ib, Ia-1, Ib-1, I-2, I-3, I-4, I-5, I-6, and I-7) and the compounds described in Tables 1A and 1B) and/or pharmaceutically acceptable salts thereof. In other embodiments, the disclosure is directed to compounds disclosed herein (e.g., Formulas A, I, I-1, Ia, Ib, Ia-1, Ib-1, I-2, I-3, I-4, I-5, I-6, and I-7) and the compounds described in Tables 1A and 1B). In yet other embodiments, the disclosure is directed to non-salt forms of the compounds disclosed herein (e.g., Formulas A, I, I-1, Ia, Ib, Ia-1, Ib-1, I-2, I-3, I-4, I-5, I-6, and I-7) and the compounds described in Tables 1A and 1B).

### Methods of Treatment

One aspect of the invention provides compounds, compositions, and methods useful for preventing or treating a disease which is at least partially mediated by orexin receptor 2. In certain embodiment, the compounds act as agonists of orexin receptor 2. In other embodiments, the compounds act as antagonists of orexin receptor 2.

Another aspect of the invention relates to methods of preventing or treating a disease selected from the group consisting of narcolepsy (e.g., narcolepsy type 1 and/or narcolepsy type 2), idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Nicmann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g. malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury, in a subject in need thereof comprising administering to the subject an effective amount of a compound disclosed herein. In certain embodiments, the method comprises administering to the subject an effective amount of a compound of Formula (I).

In certain embodiments, the invention relates to methods of treating or preventing a disease selected from the group consisting of narcolepsy, idiopathic hypersomnia, hypersomnia, sleep apnea syndrome, narcolepsy syndrome involving narcolepsy-like symptoms, hypersomnia associated with Parkinson's disease, and hypersomnia associated with dementia with Lewy body in a subject in need thereof comprising administering to the subject an effective amount of a compound disclosed herein. In certain embodiments, the method comprises administering to the subject an effective amount of a compound of Formula (I).

In certain embodiments, the disease is narcolepsy. In particular embodiments, the disease is narcolepsy type 1.

In certain embodiments, the disease is hypersomnolence.

In certain embodiments, the disease is idiopathic hypersomnia.

In certain embodiments, the disease is hypersomnia.

In certain embodiments, the disease is sleep apnea syndrome.

In certain embodiments, the disease is narcolepsy syndrome involving narcolepsy-like symptoms.

In certain embodiments, the disease is hypersomnia associated with Parkinson's disease.

In certain embodiments, the disease is hypersomnia associated with dementia with Lewy body.

In some embodiments, the compound or deuterated derivative, or pharmaceutically acceptable salt or prodrug thereof, is administered orally to the subject. In certain embodiments, the compound is administered orally to the subject.

In some embodiments, the compound or deuterated derivative, or pharmaceutically acceptable salt or prodrug thereof, is administered parenterally to the subject. In certain embodiments, the compound is administered parenterally to the subject.

In certain embodiments, the disease is prevented. In other embodiments, the disease is treated.

In certain embodiments of any one of the disclosed methods, the compound of Formula (I) is a compound of Table 1.

In some embodiments, the compounds disclosed herein (e.g., Formulas A, I, I-1, Ia, Tb, Ia-1, Ib-1, I-2, I-3, I-4, I-5, I-6, and I-7) and the compounds described in Tables 1A and 1B), deuterated derivatives of those compounds, and pharmaceutically acceptable salts or prodrugs thereof, modulate orexin 2 receptor in the subject. In other embodiments, the compounds disclosed herein (e.g., Formulas A, I, I-1, Ia, Ib, Ia-1, Ib-1, I-2, I-3, I-4, I-5, I-6, and I-7) and the compounds described in Tables 1A and 1B), deuterated derivatives of those compounds, and pharmaceutically acceptable salts or prodrugs thereof, activates orexin 2 receptor in the subject.

In certain embodiments, the compound of Formula (I) activates orexin receptor 2 in the subject.

### Pharmaceutical Compositions, Routes of Administration, and Dosing

In certain embodiments, the invention is directed to a pharmaceutical composition, comprising a compound of the invention or deuterated derivative, or a pharmaceutically acceptable salt or prodrug thereof, e.g., a compound of Formula (I), and a pharmaceutically acceptable carrier.

In certain embodiments, the invention is directed to a pharmaceutical composition, comprising a compound of any one of the disclosed embodiments, and a pharmaceutically acceptable carrier.

In some embodiments, the invention is directed to a pharmaceutical composition, comprising one or more of the compounds disclosed herein (e.g., Formulas A, I, I-1, Ia, Tb, Ia-1, Ib-1, I-2, I-3, I-4, I-5, I-6, and I-7) and the compounds described in Tables 1A and 1B), deuterated derivatives of those compounds, and pharmaceutically acceptable salts or prodrugs thereof, and a pharmaceutically acceptable carrier. In certain embodiments, the invention is directed to a pharmaceutical composition, comprising a compound of Tables 1A or 1B, or deuterated derivative, or a pharmaceutically acceptable salt or prodrug thereof, and a pharmaceutically acceptable carrier. In certain embodiments, the invention is directed to a pharmaceutical composition, comprising a compound of Table 1 or deuterated derivative, or a pharmaceutically acceptable salt or prodrug thereof, and a pharmaceutically acceptable carrier.

In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. Oral doses in the range of 0.5 to 50 milligrams/kg, in one or more administrations per day, may yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

A coating impermeable to at least pH 5.0 may ensure full gastric resistance. Examples of the more common inert ingredients that are used as enteric coatings include cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing a compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (m), most preferably 0.5 to 5 m, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulfuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, non-erodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bio-erodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

### Synthesis of Intermediate 1: 4-(2-benzyloxyphenyl)cyclohexanol

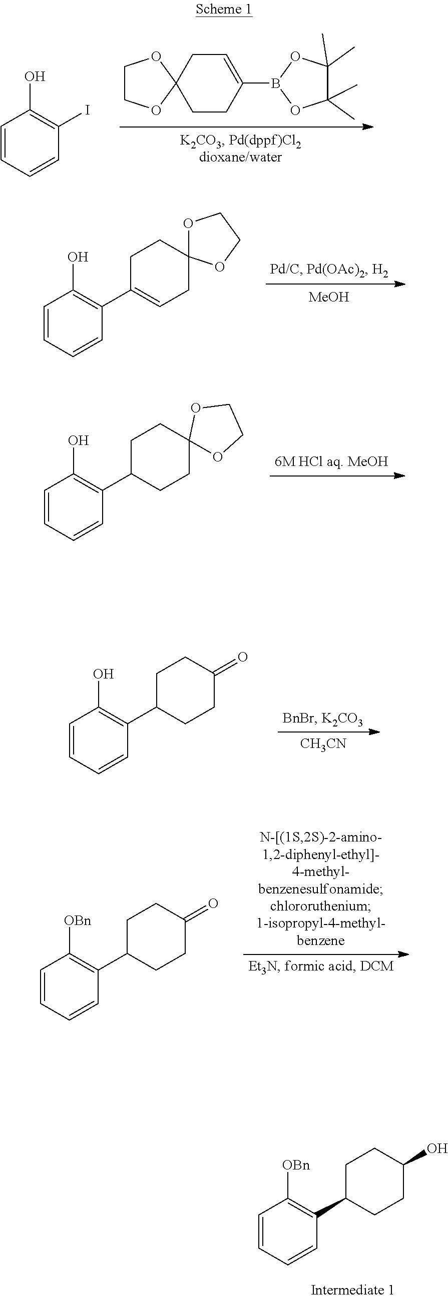

### Step 1: Preparation of 2-(1,4-dioxaspiro[4.5]dec-7-en-8-yl)phenol

2-iodophenol (200 g, 909.06 mmol, 102.56 mL, 1 eq) was charged to a 2500 mL glass flask, and then dioxane (1400 mL) and H2O (140 mL) was added at 25° C. 2-(1,4-dioxaspiro[4.5]dec-7-en-8-yl)-4,4,5,5-tetramethyl-1,3,2-dioxaborolane (290.33 g, 1.09 mol, 1.2 eq), potassium carbonate (251.28 g, 1.82 mol, 2 eq) and Pd(dppf)Cl₂ (6.65 g, 9.09 mmol, 0.01 eq) were added, then nitrogen was bubble through the reaction mixture for 15 minutes, after which the reaction was stirred at 90° C. for 12 h. Three parallel reactions were conducted. The reactions were then cooled to room temperature, combined, then were diluted with water (1800 mL) and extracted with ethyl acetate (3×2000 mL). The combined organic layers were then washed with brine (2×1500 mL), dried over sodium sulfate, filtered, and concentrated. The resulting residue was purified by silica gel chromatography (5-7% gradient ethyl acetate in petroleum ether). The isolated product was triturated with a 20:1 mixture of petroleum ether and ethyl acetate (1500 mL) at room temperature for 1.5 h. The resulting mixture was filtered, and the filter cake was dried to provide the title compound as a white solid (550 g). ¹H NMR (400 MHz, CDCl₃) δ 7.20-7.07 (m, 2H), 6.96-6.82 (m, 2H), 5.85-5.75 (m, 1H), 5.70-5.48 (m, 1H), 4.04 (s, 4H), 2.55 (t, J=7 Hz, 2H), 2.50-2.42 (m, 2H), 1.94 (t, J=7 Hz, 2H).

### Step 2: Preparation of 2-(1,4-dioxaspiro[4.5]decan-8-yl)phenol

2-(1,4-dioxaspiro[4.5]dec-7-en-8-yl)phenol (91 g, 391.78 mmol, 1 eq) was added to 1.2 L of methanol, then palladium on carbon (9.1 g, 10% Pd on carbon, 50% in water) and palladium (II) acetate (1.82 g, 8.11 mmol, 0.0207 eq) were added under a nitrogen atmosphere. The reaction vessel was then purged with hydrogen, and stirred under hydrogen (50 Psi) at room temperature for 12 h. Four parallel reactions were conducted. The reactions were then combined, filtered through celite, then the combined filtrate was concentrated. The crude product was then triturated with a 20:1 mixture of petroleum ether and ethyl acetate at room temperature for 1.5 h. The resulting mixture was filtered, and the filter cake was dried to provide the title compound as a white solid (285 g, 1.22 mol, 77.62% yield). ¹H NMR (400 MHz, CDCl₃) δ 7.22 (d, J=8 Hz, 1H), 7.06 (t, J=8 Hz, 1H), 6.90 (t, J=8 Hz, 1H), 6.75 (d, J=8 Hz, 1H), 5.75-5.30 (m, 1H), 4.02 (s, 4H), 2.96 (s, 1H), 1.92-1.85 (m, 4H), 1.82-1.69 (m, 4H).

### Step 3: Preparation of 4-(2-hydroxyphenyl)cyclohexanone

2-(1,4-dioxaspiro[4.5]decan-8-yl)phenol (94 g, 401.21 mmol, 1 eq) was dissolved in 1.2 L of methanol, then hydrochloric acid (6 M, 768.99 mL, 11.5 eq) was added. The resulting solution was stirred at room temperature for 12 h under a nitrogen atmosphere. Three parallel reactions were conducted. The reaction mixtures were then combined and concentrated under reduced pressure to remove methanol. The resulting mixture was then filtered, after which the filter cake was collected and triturate with a 20:1 mixture of petroleum ether and ethyl acetate at room temperature for 1.5 h. This mixture was then filtered and the filter cake dried under reduced pressure to provide the title compound as a white solid (182 g, 956.70 mmol, 79.48% yield). ¹H NMR (400 MHz, CDCl₃) δ 7.17 (d, J=8 Hz, 1H), 7.11 (t, J=8 Hz, 1H), 6.93 (t, J=8 Hz, 1H), 6.77 (d, J=8 Hz, 1H), 5.11 (s, 1H), 3.50-3.35 (m, 1H), 2.62-2.48 (m, 4H), 2.35-2.18 (m, 2H), 2.05-1.82 (m, 2H).

### Step 4: Preparation of 4-(2-benzyloxyphenyl)cyclohexanone

4-(2-hydroxyphenyl)cyclohexanone (91 g, 478.35 mmol, 1 eq) was added to 1 L of acetonitrile, then potassium carbonate (132.22 g, 956.70 mmol, 2 eq) and benzyl bromide (77.72 g, 454.43 mmol, 53.97 mL, 0.95 eq) were added and the reaction mixture was stirred at room temperature for 24 h under a nitrogen atmosphere. Two parallel reactions were conducted. The reactions were then combined, solids were removed by filtration, and the resulting solution was concentrated under reduced pressure. The resulting residue was triturated with a 10:1 mixture of petroleum ether and ethyl acetate at room temperature for 1 h. The resulting mixture was then filtered, then the filter cake was dried under reduced pressure to provide the title compound as a white solid (180 g, 642.03 mmol, 67.11% yield). ¹H NMR (400 MHz, CDCl₃) δ 7.55-7.32 (m, 5H), 7.25-7.16 (m, 2H), 7.02-6.89 (m, 2H), 5.14 (s, 2H), 3.61-3.44 (m, 1H), 2.61-2.41 (m, 4H), 2.30-2.16 (m, 2H), 2.00-1.85 (m, 2H).

### Step 5: Preparation of 4-(2-benzyloxyphenyl)cyclohexanol (Intermediate 1)

A solution of triethylamine (75.07 g, 741.90 mmol, 103.26 mL, 2.6 eq) in dichloromethane (700 mL) was cooled to 0° C., then formic acid (31.52 g, 684.83 mmol, 25.84 mL, 2.4 eq) and N-[(1S,2S)-2-amino-1,2-diphenyl-ethyl]-4-methyl-benzenesulfonamide;chlororuthenium;1-isopropyl-4-methyl-benzene (909.14 mg, 1.43 mmol, 0.005 eq) were added. The reaction was then warmed to room temperature and stirred for 30 min under a nitrogen atmosphere. 4-(2-benzyloxyphenyl)cyclohexanone (80 g, 285.35 mmol, 1 eq) was then added and the reaction mixture was stirred at 50° C. for 3 h under a nitrogen atmosphere. Two parallel reactions were conducted. The reactions were then combined and concentrated under reduced pressure. The resulting residue was diluted with water (2000 mL), then extracted with ethyl acetate (3×1600 mL). The combined organic layers were then washed with brine (2×1000 mL), dried over sodium sulfate, then filtered and concentrated under reduced pressure. The crude product was then purified by silica gel chromatography (20-33% gradient of ethyl acetate in petroleum ether) to provide the title compound as a white solid (105 g, 371.84 mmol, 65.16% yield). LCMS (ESI): m/z [M+H]⁺ calcd for C₁₉H₂₃O₂: 283.2, found 265.2 (M-OH). ¹H NMR (400 MHz, DMSO-d₆) δ 7.50-7.37 (m, 4H), 7.35-7.27 (m, 1H), 7.19 (d, J=8 Hz, 1H), 7.16-7.09 (m, 1H), 7.00 (d, J=8 Hz, 1H), 6.95-6.82 (m, 1H), 5.12 (s, 2H), 4.33 (d, J=2 Hz, 1H), 3.90 (d, J=2 Hz, 1H), 3.05-2.85 (m, 1H). 1.86-1.68 (m, 4H), 1.59-1.38 (m, 4H).

### Synthesis of Intermediate 2: (2¹S,2⁴S,5²R,5³S)-5³-amino-3,8-dioaxa-5(2,1)piperidina-1(1,2)-benzena-2(1,4)cyclohexanaoctaphan-6-one

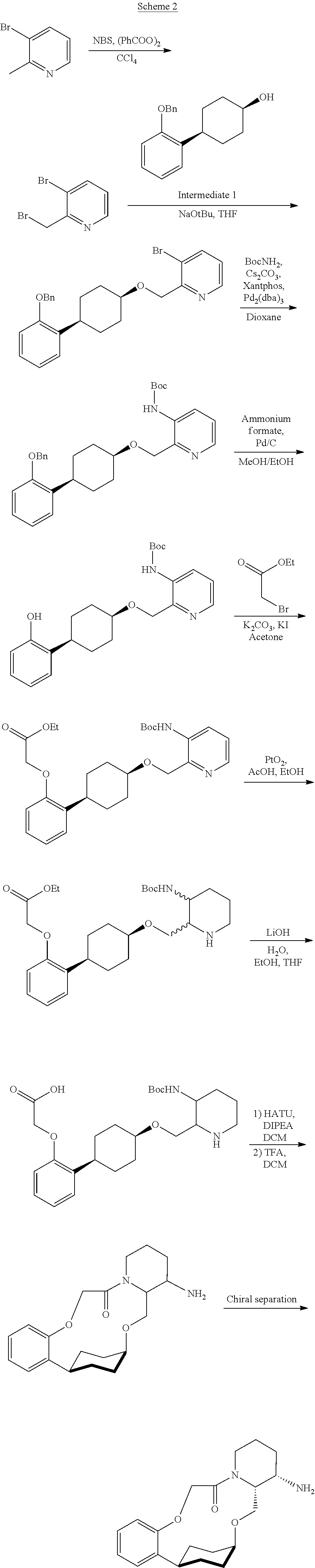

### Step 1: Preparation of 3-bromo-2-(bromomethyl)pyridine

A mixture of 2-methyl-3-bromopyridine (30.1 mL, 256 mmol), Luperox A98, benzoyl peroxide, (3.17 g, 12.8 mmol), and N-bromosuccinimide (50.2 g, 282 mmol) in carbon tetrachloride (427 mL) was degassed with nitrogen for 15 min then the solution was heated under reflux (85° C.) for 18 h. The reaction mixture was cooled to room temperature and filtered. The residue was washed with 10% EtOAc/Heptanes. The filtrate was concentrated and purified by flash column chromatography (silica gel, 0-15% EtOAc/Heptanes) to provide the title compound as a purple oil which solidified upon storage in the fridge (67.0 g, 67% yield). LCMS (ESI): m/z [M+H]⁺ calcd for C₆H₅Br₂N: 253.87, found 253.9.

### Step 2: Preparation of 2-((((1s,4s)-4-(2-(benzyloxy)phenyl)cyclohexyl)oxy)methyl)-3-bromopyridine

Under nitrogen to a solution of (1s,4s)-4-(2-(benzyloxy)phenyl)cyclohexanol (Intermediate 1) (15.0 g, 53.1 mmol) in THF (150 mL) was added sodium tert-butoxide (10.5 g, 106 mmol) and the solution was stirred at room temperature for 20 min. 3-bromo-2-(bromomethyl)pyridine (20.0 g, 79.7 mmol) in THF (45.3 mL) was added to the solution slowly and stirred at room temperature for 1 h. The reaction was quenched with saturated aqueous ammonium chloride solution (100 mL) and extracted with ethyl acetate (200 mL). The organic layer was washed with brine (30 mL), dried over sodium sulfate, filtered, and concentrated to give the title compound (26.0 g, 108% yield), which was used without further purification in the next step. LCMS (ESI): m/z [M+H]⁺ calcd for C₂₅H₂₆BrNO₂: 452.11, found 452.2.

### Step 3: Preparation of tert-butyl (2-((((1s,4s)-4-(2-(benzyloxy)phenyl)cyclohexyl)oxy)methyl)pyridin-3-yl)carbamate

Under nitrogen, to a solution of 2-((((1s,4s)-4-(2-(benzyloxy)phenyl)cyclohexyl)oxy)methyl)-3-bromopyridine (26.0 g, 57.5 mmol) in dioxane (160 mL) were added cesium carbonate (26.2 g, 80.5 mmol) and tert-butyl carbamate (10.3 g, 86.2 mmol). Nitrogen was bubbled for 15 min, 4,4-bis(diphenylphosphino)-9,9-dimethylxanthene (3.39 g, 5.75 mmol) and tris(dibenzylideneacetone)-dipalladium(0) (2.69 g, 2.87 mmol) were added. The bubbling continued for 5 min then the solution was stirred under nitrogen at 100° C. for 17 h. The reaction was diluted with ethyl acetate (200 mL)/brine (100 mL). The reaction was filtered and the organic layer was washed with brine, dried over sodium sulfate, filtered, and concentrated. The crude was purified by flash column chromatography (silica, 0-70% EtOAc/Heptanes) to give the title compound with 70% purity (27.0 g, 96% yield). LCMS (ESI): m/z [M+H]⁺ calcd for C₃₀H₃₆N₂O₄: 489.27, found 489.5.

### Step 4: Preparation of tert-butyl (2-((((1s,4s)-4-(2-hydroxyphenyl)cyclohexyl)oxy)methyl)pyridin-3-yl)carbamate

To a solution of tert-butyl (2-((((1s,4s)-4-(2-(benzyloxy)phenyl)cyclohexyl)oxy)methyl)pyridin-3-yl)carbamate (27.0 g, 55.3 mmol) in ethanol (216 mL) and methanol (216 mL) was added ammonium formate (52.3 g, 829 mmol) followed by palladium 10% on carbon wet (2.64 g, 24.8 mmol). The reaction mixture was stirred at 70° C. for 2 h. The heating was stopped and the reaction was stirred at room temperature for 2 days. The reaction was filtered through Celite, washed with methanol and the filtrate was concentrated. The crude was diluted with water/EtOAc (300 mL/500 mL) and stirred at room temperature for 10 min. The organic layer was separated, washed with brine, dried over sodium sulfate, filtered and concentrated to give the title compound used crude as such for the next step (21.9 g, 98% yield). LCMS (ESI): m/z [M+H]⁺ calcd for C₂₃H₃₀N₂O₄: 399.22, found 399.4.

### Step 5: Preparation of Ethyl 2-(2-((1s,4s)-4-((3-((tert-butoxycarbonyl)amino)pyridin-2-yl)methoxy)cyclohexyl)phenoxy)acetate

To tert-butyl (2-((((1s,4s)-4-(2-hydroxyphenyl)cyclohexyl)oxy)methyl)pyridin-3-yl)carbamate (23.2 g, 58.3 mmol) in acetone (194 mL) were added potassium carbonate (24.2 g, 175 mmol) and potassium iodide (494 mg, 2.92 mmol) followed by ethyl bromoacetate (8.6 mL, 76.0 mmol) dropwise. The solution was stirred at 50° C. for 21 h. The solution was cooled down to room temperature and the precipitate was filtered off, rinsed with acetone and concentrated. The crude was purified by flash column chromatography (silica gel, 0%-80% EtOAc/heptane) to give the title compound as a brown oil (17.9 g, 63% yield). LCMS (ESI): m/z [M+H]⁺ calcd for C₂₇H₃₆N₂O₆: 485.26, found 485.5.

### Step 6: Preparation of Ethyl 2-(2-((1s,4s)-4-((3-((tert-butoxycarbonyl)amino)piperidin-2-yl)methoxy)cyclohexyl)phenoxy)acetate

To a solution of ethyl 2-(2-((1s,4s)-4-((3-((tert-butoxycarbonyl)amino)pyridin-2-yl)methoxy)cyclohexyl)phenoxy)acetate (17.9 g, 36.9 mmol) in a mixture of ethanol (160 mL) and acetic acid (18.0 mL) under nitrogen was added platinum (IV) oxide (2.09 g, 9.22 mmol). The reaction was submitted to 5 vacuum/hydrogen cycles and then left under hydrogen for 21 h. Hydrogen was replaced by nitrogen with 5 vacuum/nitrogen cycles. The solution was filtered through Celite, rinsed well with ethanol, dichloromethane, and ethyl acetate, then concentrated to dryness. The crude was purified by flash column chromatography (silica gel, 0%-8% MeOH/DCM), fractions corresponding to cis product were concentrated to give the title compound as a mixture of enantiomers (10.7 g, 59% yield). LCMS (ESI): m/z [M+H]⁺ calcd for C₂₇H₄₂N₂O₆: 491.3, found 491.5.

### Step 7: Preparation of 2-(2-((1s,4s)-4-((3-((tert-butoxycarbonyl)amino)piperidin-2-yl)methoxy)cyclohexyl)phenoxy)acetic acid

To a solution of ethyl 2-(2-((1s,4s)-4-((3-((tert-butoxycarbonyl)amino)piperidin-2-yl)methoxy)cyclohexyl)phenoxy)acetate (10.7 g, 21.9 mmol) in a mixture of ethanol (27.0 mL), water (27.0 mL) and tetrahydrofuran (54.0 mL) was added lithium hydroxide (920.4 mg, 37.7 mmol) and the solution was stirred under nitrogen for 5.5 h then the reaction was concentrated to remove most of the organic solvent. The slurry was diluted with acetonitrile (100 mL). 1 M aqueous hydrochloric acid solution (15.8 mL, 15.8 mmol) was added dropwise and the resulting suspension was concentrated again. More acetonitrile (50 mL) was added and the suspension was concentrated again. To the residue was added water (75 mL) and acetonitrile (20 mL), frozen and lyophilized to give the crude title compound as an off-white solid (10.1 g, 100% yield). LCMS (ESI): m/z [M+H]⁺ calcd for C₂₅H₃₈N₂O₆: 463.27, found 463.5.

### Step 8: Preparation of tert-butyl ((2'S,2⁴S,5²R,5³S)-6-oxo-3,8-dioxa-5(2,1)piperidina-1(1,2)-benzena-2(1,4)cyclohexanacyclooctaphane-5³-yl)carbamate

To a solution of 2-(2-((1s,4s)-4-((3-((tert-butoxycarbonyl)amino)piperidin-2-yl)methoxy)cyclohexyl)phenoxy)acetic acid (1.00 g, 2.16 mmol) in dichloromethane (216 mL) under nitrogen at 0° C. was added N,N-diisopropylethylamine (1.51 mL, 8.65 mmol) followed by addition of HATU (1.26 g, 3.24 mmol) in one portion. The solution was stirred under nitrogen for 18 h with the ice-water bath that slowly warmed up to room temperature. Saturated aqueous sodium bicarbonate solution (200 mL) was added, phases were separated, the aqueous phase was extracted with dichloromethane (100 mL), and combined organic layers were dried over sodium sulfate, filtered and concentrated to give crude title compound used as such for the next step.

### Step 9: Preparation of (21S,2⁴S,5²R,5³S)-5³-amino-3,8-dioaxa-5(2,1)piperidina-1(1,2)-benzena-2(1,4)cyclohexanaoctaphan-6-one (Intermediate 2)

To a solution of tert-butyl ((2¹S,2⁴S,5²R,5³S)-6-oxo-3,8-dioxa-5(2,1)piperidina-1(1,2)-benzena-2(1,4)cyclohexanacyclooctaphane-5³-yl)carbamate (960 mg, 2.16 mmol) in dichloromethane (10.8 mL) was added trifluoroacetic acid (6 mL, 78.2 mmol) and the mixture was stirred at room temperature for 20 min. The reaction was concentrated to dryness, 30 mL of toluene was added and the solution was concentrated again. The crude was purified by flash column chromatography (silica gel, 25%-100% of 10% MeOH/DCM with 1% of NH₄OH in DCM to give the racemate of the title compound as a beige solid (618 mg, 83% yield). The above racemate was further purified by chiral chromatography (Analytical Column: ChiralPak IA, 250 mm×4.6 mm ID, 5 μm, Mobile Phase: 8:8:84 MeOH:EtOH:Hexane, 0.1% DEA; isocratic Flow: 1 mL/min, column temperature: 26° C., run time: 18 min; wavelength: 220 nm) to afford the title compound (190 mg, 31% yield). LCMS (ESI): m/z [M+H]⁺ calcd for C₂₀H₂₈N₂O₃: 345.21, found 345.3. ¹H NMR (400 MHz, DMSO-d₆) δ 7.13 (td, J=7.7, 1.7 Hz, 1H), 7.05 (dd, J=7.3, 1.6 Hz, 1H), 6.81 (t, J=7.5 Hz, 2H), 5.26 (d, J=10.4 Hz, 1H), 4.85 (dd, J=10.7, 5.0 Hz, 1H), 3.91 (d, J=10.4 Hz, 1H), 3.79-3.72 (m, 1H), 3.61 (d, J=18.6 Hz, 2H), 3.42 (dd, J=9.1, 3.8 Hz, 1H), 3.22 (s, 1H), 2.85-2.93 (m, 1H), 2.62 (dt, J=12.6, 10.1 Hz, 2H), 2.23 (dt, J=12.2, 8.8 Hz, 1H), 2.08 (d, J=11.2 Hz, 1H), 1.69-1.10 (m, 10H).

### Preparation of Compound 1: 1-((((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)methyl)cyclopropane-1-carbonitrile

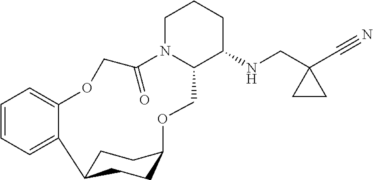 [SMILES: N#CC1(CN[C@H]2CCCN3C(=O)COc4ccccc4[C@H]4CC[C@H](CC4)OC[C@@H]23)CC1]

To a solution of Intermediate 2 (60.0 mg, 174 μmol) in dichloromethane (1.8 mL) were added 1-formylcyclopropane-1-carbonitrile (34.9 mg, 348 mol), acetic acid (36.0 μL, 628 μmol) 4 Å molecular sieves. The mixture was stirred at room temperature for 1 h and sodium cyanoborohydride (34.6 mg, 522 μmol) was added. The mixture was stirred at room temperature for 1 h. The crude was filtered, rinsed with dichloromethane and methanol, and concentrated. The residue was redissolved in methanol and purified by reverse phase chromatography (C18, 25-80% MeCN/10 mM ammonium bicarbonate buffer) to provide the title compound (51.5 mg, 70% yield). LCMS (ESI): m/z [M+H]⁺ calcd for C₂₅H₃₃N₃O₃: 424.26, found 424.3. ¹H NMR (400 MHz, CD₃OD) δ 7.14 (td, J=7.7, 1.7 Hz, 1H), 7.07 (dd, J=7.4, 1.6 Hz, 1H), 6.90-6.80 (m, 2H), 5.29 (d, J=10.6 Hz, 1H), 5.24-5.14 (m, 1H), 4.11 (d, J=10.6 Hz, 1H), 3.98-3.88 (m, 1H), 3.85-3.74 (m, 1H), 3.69 (s, 1H), 3.56-3.40 (m, 2H), 2.99-2.87 (m, 2H), 2.81-2.66 (m, 2H), 2.63-2.50 (m, 1H), 2.41-2.23 (m, 1H), 2.17 (d, J=13.2 Hz, 1H), 1.94-1.73 (m, 3H), 1.70-1.17 (m, 9H), 1.08-0.89 (m, 2H).

### Preparation of Compound 2 and Compound 3: (2¹S,2⁴S,5²R,5³S)-5³-(((R)-1,1,1-trifluoropropan-2-yl)amino)-3,8-dioxa-5(2,1)-piperidina-1(1,2)-benzena-2(1,4)-cyclohexanacyclooctaphan-6-one and (2¹S,2⁴S,5²R,5³S)-5³-(((S)-1,1,1-trifluoropropan-2-yl)amino)-3,8-dioxa-5(2,1)-piperidina-1(1,2)-benzena-2(1,4)-cyclohexanacyclooctaphan-6-one

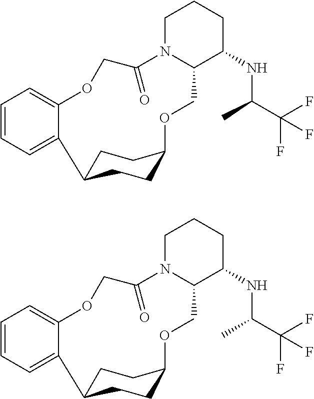 [SMILES: C[C@@H](N[C@H]1CCCN2C(=O)COc3ccccc3[C@H]3CC[C@H](CC3)OC[C@@H]12)C(F)(F)F ; C[C@H](N[C@H]1CCCN2C(=O)COc3ccccc3[C@H]3CC[C@H](CC3)OC[C@@H]12)C(F)(F)F]

To a solution of Intermediate 2 (25 mg. 72.6 μmol) and 1,1,1-trifluoroacetone (7.8 μL, 87.1 μmol) in dichloromethane (0.75 mL) was added acetic acid (75 μL). The mixture was stirred at room temperature for 1 h with 100 mg of molecular sieve 4 Å before sodium cyanoborohydride (14.4 mg, 218 μmol) was added. The reaction was stirred at room temperature for an additional 20 min before being quenched with methanol, filtered through Celite and concentrated. The crude was purified by flash column chromatography (C18, 0-100% MeCN/10 mM ammonium formate buffer) to provide a mixture of isomers which was further purified by chiral chromatography (ChiralPak IA, 250 mm×4.6 mm ID, 5 μm, 1:3:96 MeOH:EtOH:Hexane+0.1% diethylamine, flow 1 mL/min, pressure 51 bars, temp. 26° C., 16 min run) to provide Compound 2 as the first eluting isomer (9.6 mg, 30% yield). LCMS (ESI): m/z [M+H]⁺ calcd for C₂₃H₃₁F₃N₂O₃: 441.23, found 441.4. ¹H NMR (400 MHz, DMSO-d₆) δ 7.13 (dd, J=7.4, 5.9 Hz, 1H), 7.08-7.03 (m, 1H), 6.81 (t, J=7.7 Hz, 2H), 5.26 (d, J=10.4 Hz, 1H), 5.10-4.93 (m, 1H), 3.92 (d, J=10.4 Hz, 1H), 3.83-3.71 (m, 1H), 3.62 (d, J=18.2 Hz, 2H), 2.86 (d, J=10.6 Hz, 1H), 2.68-2.55 (m, 1H), 2.28 (ddd, J=22.1, 10.5, 6.6 Hz. 3H), 2.08 (d, J=14.3 Hz, 1H), 1.71-1.59 (m, 3H), 1.51-1.01 (m, 12H).

Compound 3 was isolated as the second eluting isomer (2.9 mg, 9% yield). LCMS (ESI): m/z [M+H]⁺ calcd for C₂₃H₃₁F₃N₂O₃: 441.23, found 441.4. ¹H NMR (400 MHz, DMSO-d₆) δ 7.13 (d, J=7.7 Hz, 1H), 7.05 (d, J=5.8 Hz, 1H), 6.80 (d, J=8.5 Hz, 2H), 5.26 (d, J=10.4 Hz, 1H), 4.84-4.89 (m, 1H), 3.92 (d, J=10.4 Hz, 1H), 3.79-3.72 (m, 1H), 3.66-3.53 (m, 2H), 3.44-3.37 (m, 1H), 2.76-2.79 (m, 1H), 2.22-1.95 (m, 3H), 1.76-1.61 (m, 3H), 1.08-1.48 (m, 13H).

### Preparation of Compound 4: 2-(((21S,2⁴S,5²R,5³S)-6-oxo-3,8-dioxa-5(2,1)-piperidina-1(1,2)-benzena-2(1,4)-cyclohexanacyclooctaphane-5³-yl)amino)benzonitrile

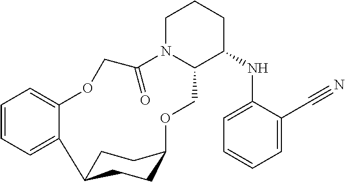 [SMILES: N#Cc1ccccc1N[C@H]1CCCN2C(=O)COc3ccccc3[C@H]3CC[C@H](CC3)OC[C@@H]12]

Intermediate 2 (20.0 mg, 58.1 μmol) was dissolved in 1,4-dioxane (0.58 mL) and 2-bromobenzonitrile (20.5 mg, 110 μmol), cesium carbonate (38.6 mg, 116 μmol) and 4,4-bis(diphenylphosphino)-9,9-dimethylxanthene (3.43 mg, 5.81 μmol) were added to the solution. The reaction was purged with nitrogen and tris(dibenzylideneacetone)-dipalladium(0) (1.03 mg, 1.12 μmol) was added and the mixture was heated at 90° C. for 18 h. The reaction was concentrated and purified by flash column chromatography (C18, 50-70% MeCN/10 mM ammonium bicarbonate buffer) to provide the title compound as a white powder (15.0 mg, 58% yield). LCMS (ESI): m/z [M+H]⁺ calcd for C₂₇H₃₁N₃O₃: 446.24, found 446.4. ¹H NMR (400 MHz, DMSO-d₆) δ 7.50 (t, J=8.5 Hz, 2H), 7.13 (t, J=7.7 Hz, 1H), 7.06 (d, J=7.6 Hz, 1H), 6.89 (d, J=8.4 Hz, 1H), 6.84-6.80 (m, 2H), 6.72 (t, J=7.5 Hz, 1H), 5.70 (d, J=7.3 Hz, 1H), 5.30 (d, J=10.4 Hz, 1H), 5.20-5.11 (m, 1H), 3.93 (dd, J=24.1, 10.5 Hz, 2H), 3.71 (d, J=10.5 Hz, 2H), 3.58 (s, 1H), 3.19 (dd, J=23.0, 16.9 Hz, 2H), 2.68-2.57 (m, 1H), 2.25-2.06 (m, 2H), 1.91 (dd, J=24.0, 12.7 Hz, 1H), 1.79 (d, J=10.9 Hz, 2H), 1.63-1.52 (m, 2H), 1.39-1.06 (m, 5H).

### Preparation of Compound 5: (2¹S,2⁴S,5²R,5³S)-5³-(((tetrahydrofuran-2-yl)methyl)amino)-3,8-dioxa-5(2,1)-piperidina-1(1,2)-benzena-2(1,4)-cyclohexanacyclooctaphan-6-one

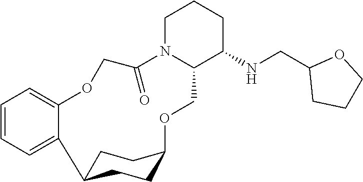 [SMILES: O=C1COc2ccccc2[C@H]2CC[C@H](CC2)OC[C@H]2[C@@H](NCC3CCCO3)CCCN12]

To a solution of Intermediate 2 (32.0 mg. 92.9 mol) in dichloromethane (0.93 mL) were added tetrahydrofuran-2-carbaldehyde (31.3 mg, 313 μmol), acetic acid (5.3 μL, 92.9 μmol) and 4 Å molecular sieves, and the mixture was stirred at 26° C. for 2 h. Sodium triacetoxyborohydride (39.4 mg, 186 μmol) was added and the solution was stirred at 27° C. for 18 h. The crude was filtered, rinsed with dichloromethane and methanol and concentrated. The residue was dissolved in N,N-dimethylformamide and purified by preparative HPLC-MS (40-60% MeCN/10 mM ammonium bicarbonate buffer) to provide the title compound as a mixture of diastereomers (10.3 mg, 26% yield). LCMS (ESI): m/z [M+H]⁺ calcd for C₂₅H₃₆N₂O₄: 429.27, found 429.5. ¹H NMR (400 MHz, CD₃OD) δ 7.18-7.12 (m, 1H), 7.08 (dd, J=7.4, 1.7 Hz, 1H), 6.86 (td, J=7.4, 1.1 Hz, 1H), 6.80 (dd, J=8.1, 1.0 Hz, 1H), 5.17 (d, J=10.3 Hz, 1H), 5.08 (dq, J=9.1, 4.5 Hz, 1H), 4.03 (d, J=10.3 Hz. 1H), 3.96-3.72 (m, 3H), 3.71-3.61 (m, 3H), 3.46-3.28 (m, 2H), 2.83-2.67 (m, 4H), 2.67-2.50 (m, 2H), 2.36-2.21 (m, 1H), 1.89-1.80 (m, 2H), 1.80-1.65 (m, 4H), 1.65-1.16 (m, 8H).

### Preparation of Compound 6: (2¹S,2⁴S,5²R,5³S)-5³-((2-methoxypyridin-3-yl)amino)-3,8-dioxa-5(2,1)-piperidina-1(1,2)-benzena-2(1,4)-cyclohexanacyclooctaphan-6-one

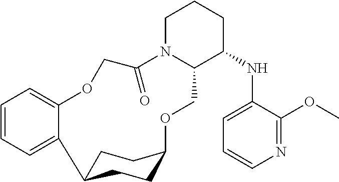 [SMILES: COc1ncccc1N[C@H]1CCCN2C(=O)COc3ccccc3[C@H]3CC[C@H](CC3)OC[C@@H]12]

Intermediate 2 (20.0 mg, 58.1 μmol) was dissolved in 1,4-dioxane (0.58 mL) and 3-bromo-2-methoxypyridine (13.8 μL, 110 μmol), cesium carbonate (38.6 mg, 116 μmol) and 4,4-bis(diphenylphosphino)-9,9-dimethylxanthene (3.43 mg, 5.81 μmol) were added to the solution. The reaction was purged with nitrogen and tris(dibenzylideneacetone)-dipalladium(0) (1.03 mg, 1.12 μmol) was added. It was then heated at 90° C. for 18 h. The reaction was concentrated and purified by flash column chromatography (C18, 45-65% MeCN/10 mM ammonium formate buffer) to provide the title compound as a white solid (8.10 mg, 31% yield). LCMS (ESI): m/z [M+H]⁺ calcd for C₂₆H₃₃N₃O₄: 452.25, found 452.4. ¹H NMR (400 MHz, CD₃OD) δ 7.40 (dd, J=5.1, 1.4 Hz, 1H), 7.14 (td, J=7.8, 1.6 Hz, 1H), 7.06 (d, J=7.5 Hz, 2H), 6.85 (td, J=7.1, 1.5 Hz, 3H), 5.42-5.35 (m, 1H), 5.32 (d, J=10.6 Hz, 1H), 4.12 (d, J=10.6 Hz, 1H), 4.03-3.98 (m, 1H), 3.96 (s, 3H), 3.84 (d, J=13.9 Hz, 1H), 3.73-3.66 (m, 1H), 3.62 (s, 1H), 3.55 (td, J=13.4, 2.5 Hz, 1H), 2.73 (qd, J=13.1, 4.5 Hz, 1H), 2.60-2.49 (m, 1H), 2.26 (ddd, J=26.1, 13.1, 4.2 Hz, 1H), 2.17 (d, J=14.0 Hz, 1H), 1.97-1.90 (m, 2H), 1.89-1.70 (m, 3H), 1.47-1.29 (m, 3H), 1.29-1.19 (m, 1H).

### Preparation of Compound 7: (2¹S,2⁴S,5²R,5³S)-5³-(pyridin-2-ylamino)-3,8-dioxa-5(2,1)-piperidina-1(1,2)-benzena-2(1,4)-cyclohexanacyclooctaphan-6-one

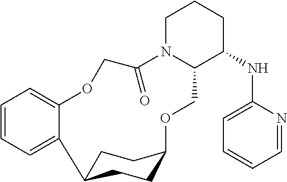 [SMILES: O=C1COc2ccccc2[C@H]2CC[C@H](CC2)OC[C@H]2[C@@H](Nc3ccccn3)CCCN12]

Intermediate 2 (20.0 mg, 58.1 μmol), cesium carbonate (38.6 mg, 116 μmol), 4,4-bis(diphenylphosphino)-9,9-dimethylxanthene (3.43 mg, 5.81 μmol) and tris(dibenzylideneacetone)-dipalladium(0) (2.66 mg, 2.90 μmol) were placed in a vial. 1,4-Dioxane (0.58 mL) was added and the reaction was degassed with nitrogen for 5 min. 2-bromopyridine (10.7 μL, 110 μmol) was added, the reaction was sealed and was heated at 90° C. for 20 h. The reaction was concentrated and purified by flash column chromatography (C18 silica, 50-70% MeCN/10 mM ammonium bicarbonate buffer) to provide the title compound as a white solid (5.8 mg. 24% yield). LCMS (ESI): m/z [M+H]⁺ calcd for C₂₅H₃₁N₃O₃: 422.24, found 422.4. ¹H NMR (400 MHz, CD₃OD) δ 8.01-7.96 (m, 1H), 7.45 (ddd, J=8.7, 7.1, 1.9 Hz, 1H), 7.14 (td, J=7.8, 1.7 Hz, 1H), 7.06 (dd, J=7.5, 1.4 Hz. 1H), 6.86 (dd, J=11.4, 4.4 Hz, 2H), 6.61-6.55 (m, 2H), 5.47-5.39 (m, 1H), 5.32 (d, J=10.5 Hz, 1H), 4.10 (dd, J=10.0, 7.0 Hz, 2H), 3.98 (dd, J=10.9, 9.1 Hz, 1H), 3.84 (d, J=13.2 Hz, 1H), 3.63 (d, J=3.7 Hz, 1H), 3.55 (t, J=12.9 Hz, 1H), 3.38 (dd, J=9.0, 3.9 Hz, 1H), 2.74 (qd, J=13.2, 4.4 Hz, 1H), 2.55 (ddd, J=17.3, 12.0, 5.2 Hz, 1H), 2.27 (qd, J=13.0, 4.1 Hz, 1H), 2.16 (d, J=13.9 Hz, 1H), 1.96-1.86 (m, 2H), 1.80-1.71 (m, 3H), 1.45-1.37 (m, 2H), 1.33 (dd, J=13.7, 4.3 Hz, 1H), 1.27-1.19 (m, 1H).

### Preparation of Compound 8: (21S,2⁴S,5²R,5³S)-5³-((1-methyl-2-oxo-1,2-dihydropyridin-3-yl)amino)-3,8-dioxa-5(2,1)-piperidina-1(1,2)-benzena-2(1,4)-cyclohexanacyclooctaphan-6-one

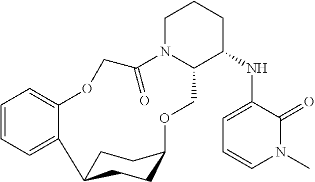 [SMILES: Cn1cccc(N[C@H]2CCCN3C(=O)COc4ccccc4[C@H]4CC[C@H](CC4)OC[C@@H]23)c1=O]

Intermediate 2 (20.0 mg, 58.1 μmol), 3-bromo-1-methylpyridin-2(1H)-one (21.8 mg, 110 μmol), cesium carbonate (38.6 mg, 116 μmol), and 4,4-bis(diphenylphosphino)-9,9-dimethylxanthene (3.43 mg, 5.81 μmol) were dissolved in anhydrous 1,4-dioxane (0.23 mL). The reaction was purged with nitrogen and tris(dibenzylideneacetone)-dipalladium(0) (1.03 mg, 1.12 μmol) was added. The reaction was then sealed under nitrogen and heated at 90° C. for 18 h. The mixture was concentrated and purified by flash column chromatography (C18 silica, 50-70% MeCN/10 mM ammonium bicarbonate buffer) to provide the title compound as a white solid (17.6 mg, 67% yield). LCMS (ESI): m/z [M+H]⁺ calcd for C₂₆H₃₃N₃O₄: 452.25, found 452.4. ¹H NMR (400 MHz, DMSO-d₆) δ 7.13 (t, J=7.7 Hz, 1H), 7.05 (d, J=7.4 Hz, 1H), 6.94 (d, J=6.7 Hz, 1H), 6.82 (dd, J=7.1, 5.6 Hz, 2H), 6.34 (d, J=7.3 Hz, 1H), 6.17 (t, J=7.0 Hz, 1H), 5.29 (d, J=10.4 Hz. 1H), 5.16 (dd, J=15.7, 7.0 Hz, 2H), 3.95 (d, J=10.4 Hz, 1H), 3.91-3.84 (m, 1H), 3.69 (d, J=13.1 Hz, 1H), 3.52 (d, J=10.3 Hz, 2H), 3.45 (s, 3H), 3.04 (dd, J=9.1, 3.4 Hz, 1H), 2.68-2.52 (m, 2H), 2.24-2.05 (m, 2H), 1.90-1.73 (m, 3H), 1.57 (d, J=12.5 Hz, 2H), 1.32 (t, J=10.7 Hz, 2H), 1.22 (dd, J=15.6, 11.8 Hz, 1H), 1.11 (d, J=11.1 Hz, 1H).

### Preparation of Compound 9: (21S,2⁴S,5²R,5³S)-5³-((2-fluorophenyl)amino)-3,8-dioxa-5(2,1)-piperidina-1(1,2)-benzena-2(1,4)-cyclohexanacyclooctaphan-6-one

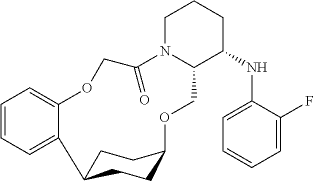 [SMILES: O=C1COc2ccccc2[C@H]2CC[C@H](CC2)OC[C@H]2[C@@H](Nc3ccccc3F)CCCN12]

Intermediate 2 (20.0 mg, 58.1 μmol), 2-bromofluorobenzene (7.0 μL, 63.9 μmol), 2,2-bis(diphenylphosphino)-1,1-binaphthyl (1.84 mg, 2.90 μmol) and sodium tert-butoxide (6.33 mg. 63.9 μmol) were placed in a vial. Toluene (0.15 mL) was added and the reaction was degassed with nitrogen for 5 min. Tris(dibenzylideneacetone)-dipalladium(0) (1.33 mg, 1.45 μmol) was added, the reaction was sealed and heated at 110° C. for 3 h. The reaction was concentrated and purified by flash column chromatography (C18 silica, 50-70% MeCN/10 mM ammonium formate buffer) to provide the title compound (12.5 mg, 49% yield). LCMS (ESI): m/z [M+H]⁺ calcd for C₂₆H₃₁FN₂O₃: 439.24, found 439.3. ¹H NMR (400 MHz, CD₃OD) δ 7.13 (t, J=7.8 Hz, 1H), 7.08-6.92 (m, 4H), 6.88-6.81 (m, 2H), 6.62 (dt, J=13.6, 4.0 Hz, 1H), 5.45-5.36 (m, 1H), 5.30 (d, J=10.6 Hz, 1H), 4.12 (d, J=10.6 Hz, 1H), 4.02-3.94 (m, 1H), 3.81 (d, J=12.2 Hz, 1H), 3.77-3.67 (m, 1H), 3.61 (s, 1H), 3.53 (dd, J=18.8, 7.6 Hz. 1H), 3.33 (dd, J=9.1, 3.9 Hz, 1H), 2.73 (qd, J=13.1, 4.4 Hz, 1H), 2.59-2.47 (m, 1H), 2.26 (qd, J=13.0, 4.1 Hz, 1H), 2.16 (d, J=13.7 Hz, 1H), 1.90 (dd, J=21.9, 9.5 Hz, 2H), 1.81 (dd, J=12.4, 3.5 Hz. 1H), 1.77-1.68 (m, 2H), 1.41 (dd, J=13.2, 3.4 Hz, 2H), 1.36-1.28 (m, 1H), 1.27-1.18 (m, 1H).

### Preparation of Compound 10: 3-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)picolinonitrile

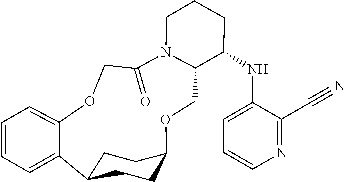 [SMILES: N#Cc1ncccc1N[C@H]1CCCN2C(=O)COc3ccccc3[C@H]3CC[C@H](CC3)OC[C@@H]12]

Intermediate 2 (30.0 mg, 87.1 μmol), 4,4-bis(diphenylphosphino)-9,9-dimethylxanthene (5.14 mg, 8.71 μmol), cesium carbonate (57.9 mg, 174 μmol) and 3-bromopyridine-2-carbonitrile (30.9 mg, 165 μmol) were placed in a vial. 1,4-Dioxane (0.87 mL) was added and the reaction was degassed with nitrogen for 5 min. Tris(dibenzylideneacetone)-dipalladium(0) (3.99 mg, 4.35 μmol) was added, the reaction was sealed and heated at 90° C. for 20 h. The reaction was purified by reverse phase flash column chromatography (C18, 40-60% MeCN/10 mM ammonium formate buffer) to provide the title compound as a white solid (21.4 mg, 55%). LCMS (ESI): m/z [M+H]⁺ calcd for C₂₆H₃₀N₄O₃: 447.24, found 447.3. ¹H NMR (400 MHz, CD₃OD) δ 7.97-7.91 (m, 1H), 7.54 (d, J=8.8 Hz, 1H), 7.51-7.45 (m, 1H), 7.14 (t, J=7.7 Hz, 1H), 7.06 (d, J=7.3 Hz, 1H), 6.86 (t, J=8.5 Hz, 2H), 5.32 (dt, J=10.2, 5.1 Hz, 2H), 4.13 (dd, J=10.6, 2.0 Hz, 1H), 4.05 (t, J=9.5 Hz, 1H), 3.86 (d, J=12.6 Hz, 2H), 3.66 (s, 1H), 3.56 (t, J=13.3 Hz, 1H), 3.37-3.32 (m, 1H), 2.71 (dt, J=22.3, 11.0 Hz, 1H), 2.56 (dd, J=14.5, 9.0 Hz, 1H), 2.32-2.13 (m, 2H), 1.96 (dd, J=13.6, 7.6 Hz, 4H), 1.75 (d, J=12.8 Hz, 2H), 1.44 (d, J=13.2 Hz, 2H), 1.40-1.30 (m, 2H), 1.24 (d, J=12.5 Hz, 1H).

### Synthesis of Compound 11: (2¹S,2⁴S,5²R,5³S)-5³-((3,3,3-trifluoro-2,2-dimethylpropyl)amino)-3,8-dioxa-5(2,1)-piperidina-1(1,2)-benzena-2(1,4)-cyclohexanacyclooctaphan-6-one

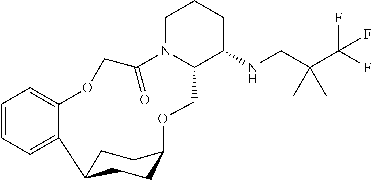 [SMILES: CC(C)(CN[C@H]1CCCN2C(=O)COc3ccccc3[C@H]3CC[C@H](CC3)OC[C@@H]12)C(F)(F)F]

### Step 1: Preparation of 3,3,3-trifluoro-2,2-dimethylpropanol

3,3,3-trifluoro-2,2-dimethylpropionic acid (300 mg, 1.86 mmol) was dissolved in diethyl ether (4.50 mL) and cooled to 0° C. Lithium aluminum hydride (1.86 mL, 3.73 mmol) was slowly added at 0° C. and the mixture was allowed to slowly warm to room temperature and stirred for 18 h. Water (1 mL) was added to quench the reaction. 2 M sodium hydroxide (2 mL) was added followed by water (1 mL) and the mixture was stirred for 15 min, then was filtered over Celite and the organic phase was partially concentrated under reduced pressure. The crude was used as such in the next step assuming quantitative yield.

### Step 2: Preparation of 3,3,3-trifluoro-2,2-dimethylpropanal

3,3,3-trifluoro-2,2-dimethylpropanol (265 mg, 1.86 mmol) was dissolved in dichloromethane (9.32 mL) and Dess-Martin periodinane (1.03 g, 2.42 mmol) was added. The mixture was stirred at room temperature for 2.5 h. To the reaction was added aqueous saturated sodium carbonate (5 mL) and 1 M sodium thiosulfate (2 mL) and was stirred for 30 min. The aqueous phase was washed with dichloromethane (2×10 mL), and the combined organic layers were partially concentrated and used as such in the next step assuming quantitative yield.

### Step 3: Preparation of (21S,2⁴S,5²R,5³S)-5³-((3,3,3-trifluoro-2,2-dimethylpropyl)amino)-3,8-dioxa-5(2,1)-piperidina-1(1,2)-benzena-2(1,4)-cyclohexanacyclooctaphan-6-one (Compound 14)

Intermediate 2 (20.0 mg, 58.1 μmol) was dissolved in methanol (0.15 mL) and a solution of crude 3,3,3-trifluoro-2,2-dimethylpropanal (8.13 mg, 58.1 μmol) in dichloromethane (500 μL) was added and was stirred for 1 h. The reaction was cooled to 0° C. and sodium borohydride (22.9 mL, 581 μmol) was added and was stirred at 0° C. for 30 min. The reaction was diluted with saturated aqueous sodium carbonate and extracted with dichloromethane (3×2 mL). The organic phase was dried with magnesium sulfate, concentrated and was purified by reverse phase chromatography (C18, 55-75% MeCN/10 mM ammonium formate buffer) to provide the title compound (9.80 mg, 36%). LCMS (ESI): m/z [M+H]⁺ calcd for C₂₅H₃₅F₃N₂O₃: 469.26, found 469.3. ¹H NMR (400 MHz, CD₃OD) δ 7.13 (td, J=7.8, 1.4 Hz, 1H), 7.06 (dd, J=7.4, 1.4 Hz. 1H), 6.88-6.79 (m, 2H), 5.28 (d, J=10.6 Hz, 1H), 5.21-5.13 (m, 1H), 4.10 (d, J=10.6 Hz, 1H), 3.95-3.87 (m, 1H), 3.77 (d, J=14.4 Hz, 1H), 3.67 (s, 1H), 3.54-3.41 (m, 2H), 2.80-2.68 (m, 4H), 2.55 (ddd, J=17.5, 12.0, 5.2 Hz, 1H), 2.30 (qd, J=13.1, 4.1 Hz, 1H), 2.16 (d, J=14.1 Hz, 1H), 1.87-1.78 (m, 3H), 1.67-1.57 (m, 1H), 1.43 (qdd, J=18.3, 15.7, 5.2 Hz, 5H), 1.30-1.23 (m, 1H), 1.15 (s, 6H).

### Preparation of Compound 12 and Compound 13: (1S,3r)-3-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)cyclobutane-1-carbonitrile and (1R,3s)-3-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)cyclobutane-1-carbonitrile

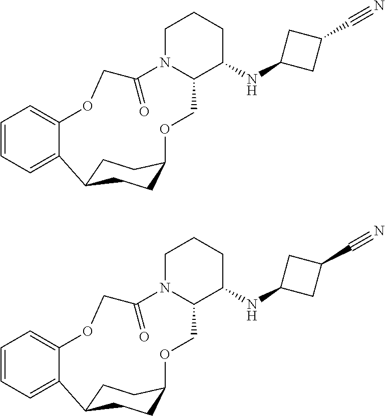 [SMILES: N#C[C@H]1C[C@H](N[C@H]2CCCN3C(=O)COc4ccccc4[C@H]4CC[C@H](CC4)OC[C@@H]23)C1 ; N#C[C@H]1C[C@@H](N[C@H]2CCCN3C(=O)COc4ccccc4[C@H]4CC[C@H](CC4)OC[C@@H]23)C1]

To a solution of Intermediate 2 (32.0 mg, 92.9 μmol) in dichloromethane (0.6 mL) were added 3-oxocyclobutanecarbonitrile (11.6 mg, 116 μmol), acetic acid (60.0 μL) and 4 Å molecular sieves. The mixture was stirred at room temperature for 1 h and sodium cyanoborohydride (11.5 mg, 174 μmol) was added. The mixture was stirred at room temperature for 1 h. The crude was filtered, rinsed with dichloromethane and methanol and the filtrate was concentrated. The residue was dissolved in methanol and purified by reverse phase chromatography (C18, 20-80% MeCN/10 mM ammonium formate buffer) to provide a mixture of isomers which was further purified by chiral chromatography (ChiralPak IA, 250 mm×4.6 mm ID, 5 μm, 5:25:75 MeOH:IPrOH:Hexane with 0.1% diethylamine, flow 0.8 mL/min, pressure at 61 bars, temp. 26° C., 15 min run) to provide Compound 12 as the first eluting isomer (2.26 mg, 9% yield). LCMS (ESI): m/z [M+H]⁺ calcd for C₂₅H₃₃N₃O₃: 424.26, found 424.3. ¹H NMR (400 MHz, CD₃OD) δ 7.19-7.11 (m, 1H), 7.07 (d, J=7.4 Hz, 1H), 6.85 (dd, J=13.6, 7.4 Hz, 2H), 5.29 (d, J=10.6 Hz, 1H), 5.15-5.05 (m, 1H), 4.10 (d, J=10.6 Hz, 1H), 3.96-3.87 (m, 1H), 3.87-3.73 (m, 2H), 3.69 (s, 1H), 3.55-3.39 (m, 2H), 3.17-3.10 (m, 1H), 2.89-2.64 (m, 2H), 2.64-2.49 (m, 3H), 2.40-2.21 (m, 2H), 2.16 (d, J=14.7 Hz, 1H), 1.89-1.75 (m, 3H), 1.68-1.37 (m, 5H), 1.36-1.23 (m, 2H), 0.89 (d, J=7.6 Hz, 1H).

Compound 13 was provided as the second eluting isomer (4.82 mg. 20% yield). LCMS (ESI): m/z [M+H]⁺ calcd for C₂₅H₃₃N₃O₃: 424.26, found 424.3. ¹H NMR (400 MHz, CD₃OD) δ 7.18-7.10 (m, 1H), 7.07 (dd, J=7.4, 1.7 Hz, 1H), 6.89-6.79 (m, 2H), 5.29 (d, J=10.6 Hz, 1H), 5.13-5.03 (m, 1H), 4.10 (d, J=10.6 Hz, 1H), 3.95-3.85 (m, 1H), 3.78 (d, J=10.8 Hz, 1H), 3.68 (s, 1H), 3.54-3.38 (m, 3H), 2.99-2.79 (m, 2H), 2.77-2.63 (m, 3H), 2.62-2.50 (m, 1H), 2.36-2.22 (m, 1H), 2.15 (ddd, J=19.2, 13.9, 8.3 Hz, 3H), 1.89-1.72 (m, 3H), 1.68-1.21 (m, 7H).

### Preparation of Compound 14: 3-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)isonicotinonitrile

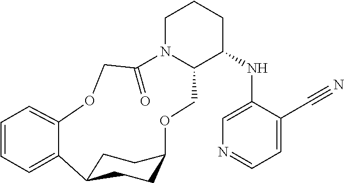 [SMILES: N#Cc1ccncc1N[C@H]1CCCN2C(=O)COc3ccccc3[C@H]3CC[C@H](CC3)OC[C@@H]12]

Intermediate 2 (30 mg, 87.1 μmol), cesium carbonate (57.9 mg, 174 μmol), 3-bromo-4-cyanopyridine (16.3 mg, 87.1 μmol), 4,4-bis(diphenylphosphino)-9,9-dimethylxanthene (5.14 mg, 8.71 μmol) and tris(dibenzylideneacetone)-dipalladium(0) (3.99 mg, 4.35 μmol) were placed in a vial. 1,4-Dioxane (0.87 mL) was added under nitrogen and the reaction was degassed for 5 min. The reaction was sealed and was heated at 90° C. for 22 h. The reaction was filtered over Celite, rinsed with methanol, concentrated, and purified by reverse phase flash column chromatography (C18, 25-100% MeCN/10 mM ammonium formate buffer) to provide the title compound as a pale yellow solid (25.1 mg, 65% yield). LCMS (ESI): m/z [M+H]⁺ calcd for C₂₆H₃₀N₄O₃: 447.24, found 447.3. ¹H NMR (400 MHz, CD₃OD) δ 8.45 (s, 1H), 7.94 (d, J=4.7 Hz, 1H), 7.44 (d, J=5.0 Hz, 1H), 7.15 (td, J=7.7, 1.7 Hz, 1H), 7.07 (dd, J=7.3, 1.6 Hz, 1H), 6.87 (dd, J=12.1, 4.6 Hz, 2H), 5.44-5.36 (m, 1H), 5.33 (d, J=10.6 Hz, 1H), 4.14 (d, J=10.6 Hz, 1H), 4.11-3.98 (m, 2H), 3.91-3.77 (m, 1H), 3.67 (s, 1H), 3.63-3.51 (m, 1H), 3.36 (dd, J=9.1, 3.7 Hz, 1H), 2.80-2.65 (m, 1H), 2.62-2.48 (m, 1H), 2.42-2.12 (m, 2H), 2.08-1.89 (m, 3H), 1.87-1.69 (m, 2H), 1.56-1.15 (m, 4H).

### Synthesis of Compound 15: (21S,2⁴S,5²R,5³S)-5³-(((1-(trifluoromethyl)cyclopropyl)methyl)amino)-3,8-dioxa-5(2,1)-piperidina-1(1,2)-benzena-2(1,4)-cyclohexanacyclooctaphan-6-one

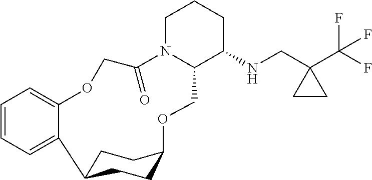 [SMILES: O=C1COc2ccccc2[C@H]2CC[C@H](CC2)OC[C@H]2[C@@H](NCC3(C(F)(F)F)CC3)CCCN12]

### Step 1: Preparation of N-methoxy-N-methyl-2-(1-(trifluoromethyl)cyclopropyl)acetamide

To a solution of 1-(trifluoromethyl)cyclopropane-1-carboxylic acid (100 mg, 649 μmol), N,O-dimethylhydroxylamine hydrochloride (64.6 mg, 649 mol), and N,N-diisopropylethylamine (126 μL, 714 μmol) in dimethylformamide (0.5 mL) was added 1-[bis(dimethylamino)methylene]-1H-1,2,3-triazolo[4,5-b]pyridinium 3-oxide hexafluorophosphate (271 mg, 714 μmol). The reaction was stirred at room temperature for 30 min then quenched with water. Ethyl acetate was added, the organic phase was washed with water, brine and dried over sodium sulfate, filtered and concentrated to give the title compound used directly in the next step.

### Step 2: Preparation of 2-(1-(trifluoromethyl)cyclopropyl)acetaldehyde

To a solution of N-methoxy-N-methyl-2-(1-(trifluoromethyl)cyclopropyl)acetamide (68.0 mg, 345 μmol) in tetrahydrofuran (2 mL) was added lithium aluminum hydride (19.7 mg, 517 μmol) at 0° C. under nitrogen. The reaction was stirred for 10 min at 0° C. before slowly warming up over 1 h. The reaction was then quenched with sodium sulfate decahydrate (100 mg) and stirred for another 20 min. The crude reaction was filtered and used directly in the next step.

### Step 3: Preparation of (21S,2⁴S,5²R,5³S)-5³-(((1-(trifluoromethyl)cyclopropyl)methyl)amino)-3,8-dioxa-5(2,1)-piperidina-1(1,2)-benzena-2(1,4)-cyclohexanacyclooctaphan-6-one (Compound 18)

To a solution of Intermediate 2 (50 mg, 145 μmol) and 2-(1-(trifluoromethyl)cyclopropyl)acetaldehyde (47.6 mg. 345 μmol) in dichloromethane (1.50 mL) was added acetic acid (83.3 μL). The mixture was stirred for 5 min at room temperature before sodium cyanoborohydride (26.6 mg, 435 μmol) was added. The reaction was stirred at room temperature for 1 h before being quenched with methanol and concentrated. The crude was purified by flash column chromatography (C18, 0-100% MeCN/10 mM ammonium formate buffer) to give to provide the title compound (14.0 mg, 21% yield). LCMS (ESI): m/z [M+H]⁺ calcd for C₂₅H₃₃F₃N₂O₃: 467.25, found 467.3. ¹H NMR (400 MHz, DMSO-d₆) δ 7.10 (ddd, J=23.1, 12.4, 7.6 Hz, 2H), 6.81 (t, J=8.4 Hz, 2H), 5.25 (d, J=10.4 Hz, 1H), 5.04-4.92 (m, 1H), 3.92 (d, J=10.4 Hz, 1H), 3.79-3.71 (m, 1H), 3.67-3.56 (m, 2H), 2.70 (ddt, J=35.8, 32.1, 13.5 Hz, 5H), 2.31-2.03 (m, 3H), 1.68 (dd, J=26.3, 14.2 Hz, 3H), 1.50-1.07 (m, 8H), 0.90-0.79 (m, 4H).

### Preparation of Compound 16: 2-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)acetamide

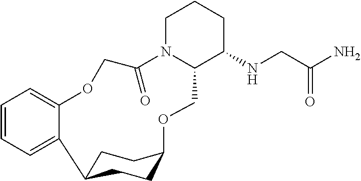 [SMILES: NC(=O)CN[C@H]1CCCN2C(=O)COc3ccccc3[C@H]3CC[C@H](CC3)OC[C@@H]12]

Intermediate 2 (20.0 mg, 58.1 μmol) was dissolved in acetonitrile (0.40 mL). N,N-diisopropylethylamine (15.3 μL, 87.1 μmol) was added followed by 2-bromoacetamide (8.17 mg, 58.1 μmol). The reaction was heated at 80° C. for 18 h, then was concentrated and purified by flash column chromatography (C18, 0-80% MeCN/10 mM ammonium formate buffer) to provide the title compound as a white solid (13.00 mg, 56% yield). LCMS (ESI): m/z [M+H]⁺ calcd for C₂₂H₃₁N₃O₄: 402.23, found 402.3. ¹H NMR (400 MHz, CD₃OD) δ 7.14 (td, J=7.8, 1.7 Hz, 1H), 7.06 (dd, J=7.4, 1.6 Hz, 1H), 6.89-6.80 (m, 2H), 5.28 (d, J=10.6 Hz, 1H), 5.20 (s, 1H), 4.10 (d, J=10.6 Hz, 1H), 3.95 (t, J=8.9 Hz, 1H), 3.79 (d, J=13.4 Hz, 1H), 3.70 (s, 1H), 3.60-3.35 (m, 4H), 2.94 (d, J=10.0 Hz, 1H), 2.72 (qd, J=12.9, 4.3 Hz, 1H), 2.61-2.51 (m, 1H), 2.29 (qd, J=13.0, 4.1 Hz, 1H), 2.16 (d, J=14.0 Hz, 1H), 2.01-1.75 (m, 3H), 1.69-1.51 (m, 2H), 1.52-1.31 (m, 4H), 1.30-1.22 (m, 1H).

### Preparation of Compound 17: 2-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)nicotinonitrile

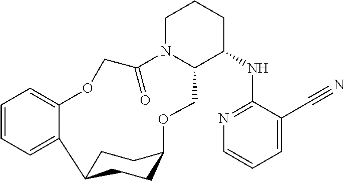 [SMILES: N#Cc1cccnc1N[C@H]1CCCN2C(=O)COc3ccccc3[C@H]3CC[C@H](CC3)OC[C@@H]12]

To a solution of Intermediate 2 (30.0 mg, 87.1 mol) and 3-cyano-2-fluoropyridine (16.3 mg, 131 μmol) in N-methyl-2-pyrrolidone (0.99 mL) was added N,N-diisopropylethylamine (46.0 μL, 261 μmol). The resulting solution was heated at 80° C. for 20 h. The crude was directly purified by flash column chromatography (C18, 5-100% MeCN/10 mM ammonium formate buffer) to provide the title compound (26.4 mg, 68% yield). LCMS (ESI): m/z [M+H]⁺ calcd for C₂₆H₃₀N₄O₃: 447.24, found 447.2. ¹H NMR (400 MHz, DMSO-d₆) δ 8.34 (dd, J=4.9, 1.9 Hz, 1H), 7.95 (dd, J=7.6, 1.9 Hz, 1H), 7.15 (td, J=7.9, 1.7 Hz, 1H), 7.11-7.04 (m, 1H), 6.89-6.79 (m, 3H), 6.73 (dd, J=7.6, 4.9 Hz, 1H), 5.32 (d, J=10.3 Hz, 1H), 5.29-5.20 (m, 1H), 4.34-4.18 (m, 1H), 3.98-3.86 (m, 2H), 3.71 (d, J=11.8 Hz, 1H), 3.60 (s, 1H), 3.22 (dd, J=9.4, 3.8 Hz, 1H), 2.69-2.58 (m, 2H), 2.27-2.03 (m, 2H), 1.96 (t, J=11.3 Hz, 1H), 1.88-1.67 (m, 2H), 1.67-1.45 (m, 2H), 1.43-1.04 (m, 4H).

### Synthesis of Compound 18: (2¹S,2⁴S,5²R,5³S)-5³-(((1-fluorocyclopropyl)methyl)amino)-3,8-dioxa-5(2,1)-piperidina-1(1,2)-benzena-2(1,4)-cyclohexanacyclooctaphan-6-one

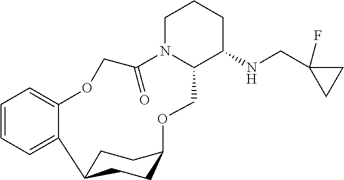 [SMILES: O=C1COc2ccccc2[C@H]2CC[C@H](CC2)OC[C@H]2[C@@H](NCC3(F)CC3)CCCN12]

### Step 1: Preparation of 2-(1-fluorocyclopropyl)-N-methoxy-N-methylacetamide

To a solution of 1-fluorocyclopropanecarboxylic acid (67.5 mg, 649 μmol), N,O-dimethylhydroxylamine hydrochloride (64.6 mg, 649 μmol), and N,N-diisopropylethylamine (126 μL, 714 μmol) in dimethylformamide (0.50 mL) was added 1-[bis(dimethylamino)methylene]-1H-1,2,3-triazolo[4,5-b]pyridinium 3-oxide hexafluorophosphate (271 mg, 714 μmol). The reaction was stirred at room temperature for 30 min then quenched with water. Ethyl acetate was added, the organic phase was washed with water, brine, dried over sodium sulfate, filtered and concentrated to give the title compound which was used directly in the next step.

### Step 2: Preparation of 2-(1-fluorocyclopropyl)acetaldehyde

To a solution of 2-(1-fluorocyclopropyl)-N-methoxy-N-methylacetamide (50.8 mg, 345 μmol) in tetrahydrofuran (2 mL) was added lithium aluminum hydride (19.7 mg, 517 μmol) at 0° C. under nitrogen. The reaction was stirred for 10 min at 0° C. before slowly warming up over 1 h. The reaction was then quenched with sodium sulfate decahydrate (100 mg) and stirred for another 20 min. The crude reaction was filtered and used directly in the next step.

### Step 3: Preparation of (21S,2⁴S,5²R,5³S)-5³-(((1-fluorocyclopropyl)methyl)amino)-3,8-dioxa-5(2,1)-piperidina-1(1,2)-benzena-2(1,4)-cyclohexanacyclooctaphan-6-one (Compound 21)

To a solution of Intermediate 2 (50 mg, 145 μmol), 2-(1-fluorocyclopropyl)acetaldehyde (20.5 mg, 232 μmol) in dichloromethane (1.50 mL) was added acetic acid (10 μL). The mixture was stirred at room temperature for 1 h with 100 mg of 4 Å molecular sieves before sodium cyanoborohydride (57.6 mg, 870 mol) was added. The reaction was stirred at room temperature for an additional 1 h before being quenched with methanol and concentrated. The crude was purified by flash column chromatography (C18, 0-100% MeCN/10 mM ammonium formate buffer) to provide the title compound (23.0 mg, 38% yield). LCMS (ESI): m/z [M+H]⁺ calcd for C₂₄H₃₃FN₂O₃: 417.25, found 417.4. ¹H NMR (400 MHz, DMSO-d₆) δ 9.04 (s, 1H), 7.24-7.04 (m, 2H), 6.95-6.78 (m, 2H), 5.32-5.25 (m, 1H), 4.00-3.44 (m, 5H), 2.66-2.61 (m, 1H), 2.38-0.07 (m, 21H).

### Preparation of Compound 19: (2¹S,2⁴S,5²R,5³S)-5³-((2-fluorobenzyl)amino)-3,8-dioxa-5(2,1)-piperidina-1(1,2)-benzena-2(1,4)-cyclohexanacyclooctaphan-6-one

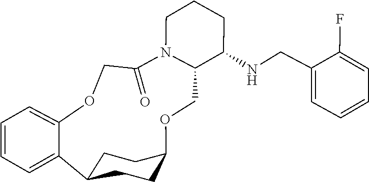 [SMILES: O=C1COc2ccccc2[C@H]2CC[C@H](CC2)OC[C@H]2[C@@H](NCc3ccccc3F)CCCN12]

To a solution of Intermediate 2 (20 mg, 58.1 mol), and 2-fluorobenzaldehyde (9.8 μL, 92.9 μmol) in dichloromethane (0.60 mL) was added acetic acid (4 L). The mixture was stirred at room temperature for 20 min before sodium cyanoborohydride (11.5 mg, 174 μmol) was added. The reaction was stirred at room temperature for an additional 20 min before being quenched with methanol and concentrated. The crude was purified by flash column chromatography (C18, 0-100% MeCN/10 mM ammonium formate buffer) to provide the title compound (25.0 mg, 93% yield). LCMS (EST): m/z [M+H]⁺ calcd for C₂₇H₃₃FN₂O₃: 453.25, found 453.5. ¹H NMR (400 MHz, DMSO-d₆) δ 7.48-4.43 (m, 1H), 7.21-7.12 (m, 4H), 7.06 (d, J=7.1 Hz, 1H), 6.81 (t, J=7.7 Hz, 2H), 5.26 (d, J=9.5 Hz, 1H), 5.05 (s, 1H), 3.97-3.58 (m, 6H), 3.45-3.40 (m, 1H), 2.80-2.62 (m, 2H), 2.33-2.01 (m, 3H), 1.78-1.61 (m, 3H), 1.42-1.16 (m, 5H).

### Preparation of Compound 20: 4-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)nicotinonitrile

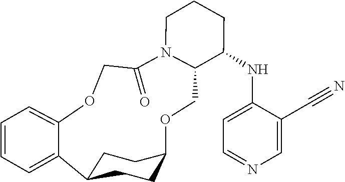 [SMILES: N#Cc1cnccc1N[C@H]1CCCN2C(=O)COc3ccccc3[C@H]3CC[C@H](CC3)OC[C@@H]12]

Intermediate 2 (30.0 mg, 87.1 μmol), N,N-diisopropylethylamine (50.0 μL, 284 μmol), and 3-bromopyridine-2-carbonitrile (20.6 mg, 110 μmol) were placed in a vial. N-Methyl-2-pyrrolidone (0.44 mL) was added, the reaction was sealed and heated at 160° C. for 1.5 h. The crude was directly purified by reverse phase chromatography (C18, 35-55% MeCN/10 mM ammonium formate buffer) to provide the title compound (22.7 mg, 58% yield). LCMS (ESI): m/z [M+H]⁺ calcd for C₂₆H₃₀N₄O₃: 447.24, found 447.3. ¹H NMR (400 MHz, CD₃OD) δ 8.41 (s, 1H), 8.28 (d, J=6.2 Hz, 1H), 7.14 (td, J=7.7, 1.6 Hz, 1H), 7.06 (dd, J=7.3, 1.6 Hz, 1H), 7.01 (d, J=6.4 Hz, 1H), 6.89-6.81 (m, 2H), 5.32 (dd, J=9.9, 6.4 Hz, 2H), 4.13 (d, J=10.7 Hz, 1H), 4.08-4.01 (m, 1H), 4.01-3.93 (m, 1H), 3.90-3.82 (m, 1H), 3.66 (s, 1H), 3.55 (td, J=13.4, 2.3 Hz, 1H), 3.32 (d, J=4.4 Hz, 1H), 2.72 (qd, J=13.0, 4.3 Hz, 1H), 2.61-2.50 (m, 1H), 2.25 (ddd, J=32.2, 20.7, 9.6 Hz, 2H), 2.09-1.89 (m, 3H), 1.84-1.69 (m, 2H), 1.48-1.30 (m, 3H), 1.29-1.20 (m, 1H).

### Synthesis of Intermediate 3: (1s,4s)-4-(2-(benzyloxy)-3,5-difluorophenyl)cyclohexan-1-ol

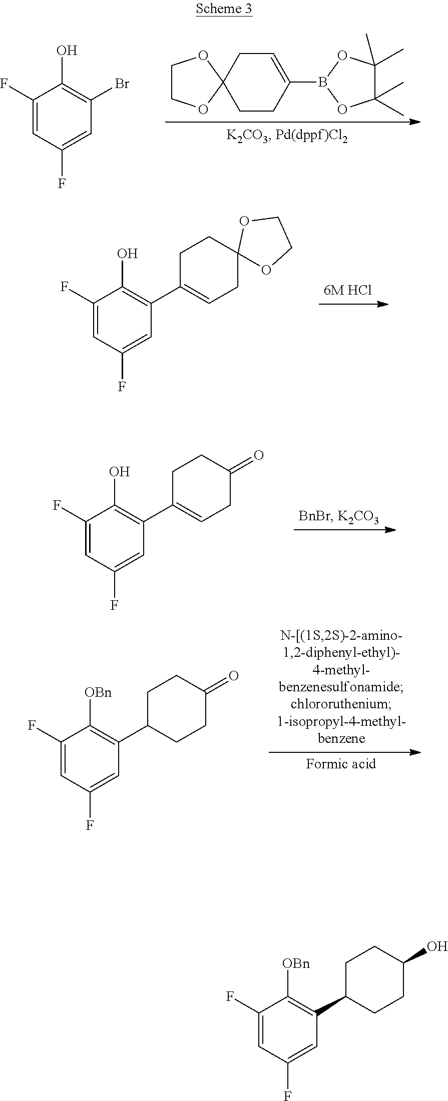 [SMILES: Oc1c(F)cc(F)cc1Br ; CC1(C)OB(C2=CCC3(CC2)OCCO3)OC1(C)C ; Oc1c(F)cc(F)cc1C1=CCC2(CC1)OCCO2 ; O=C1CC=C(c2cc(F)cc(F)c2O)CC1 ; O=C1CCC(c2cc(F)cc(F)c2OCc2ccccc2)CC1 ; O[C@H]1CC[C@@H](c2cc(F)cc(F)c2OCc2ccccc2)CC1]

### Step 1: Preparation of 2,4-difluoro-6-(1,4-dioxaspiro[4.5]dec-7-en-8-yl)phenol

The reaction vessel was charged with 2-bromo-4,6-difluorophenol (580 g, 2.26 mol), 1,4-dioxane (4060 mL), and water (406 mL). 4,4,5,5-tetramethyl-2-(1,4-dioxaspiro[4.5]dec-7-en-8-yl)-1,3,2-dioxaborolane (886 g, 2.71 mol) and potassium carbonate (767 g, 2.71 mol) were then added, followed by [1,1′-Bis(diphenylphosphino)ferrocene]dichloropalladium(II) (227 g, 0.226 mol). The reaction vessel was degassed and purged with nitrogen, then the reaction was stirred at 90° C. for 12 h. The reaction mixture was cooled to room temperature and filtered through a celite pad. Water (3 V) was added to the filtrate, then the mixture was extracted with ethyl acetate (3×5 V). The combined organic layers were washed with brine (5 V), then were dried over sodium sulfate, filtered, and concentrated under reduced pressure. The crude product was filtered through a silica pad with ethyl acetate, then the filtrate was concentrated and triturated with 20:1 petroleum ether/ethyl acetate (2.5 V); the resulting mixture was stirred for 16 h. The mixture was then filtered and the filter-cake was washed with 20:1 petroleum ether/ethyl acetate (2.5 V). The filter-cake was then dried under vacuum to provide the title compound as a dark grey solid (715.5 g, 90.8% purity, 59.2% yield). ¹H NMR (400 MHz, CDCl₃) δ 6.73 (m, 2H), 5.86 (s, 1H), 5.29 (s, 1H), 4.03 (s, 4H), 2.58 (m, 2H), 2.47 (m, 2H), 1.92 (m, 2H).

### Step 2: Preparation of 2,4-difluoro-6-(1,4-dioxaspiro[4.5]decan-8-yl)phenol

The reaction vessel was charged with 10% palladium on carbon (50% w/w water, 8.35 g) and methanol (1000 mL). 2,4-difluoro-6-(1,4-dioxaspiro[4.5]dec-7-en-8-yl)phenol (100 g, 0.373 mol) was then added followed by palladium (11) acetate (1.67 g, 7.56 mmol) and the reaction vessel was degassed and purged with hydrogen. The reaction mixture was then stirred at room temperature for 5 days under hydrogen (50 Psi), while monitoring for consumption of starting material. This procedure was carried out across seven batches. The batches were then combined and filtered through a celite pad and the filtrate was concentrated to provide crude product as a yellow solid (870 g). The crude product was triturated with 20:1 petroleum ether/ethyl acetate (3 V), then was filtered. Filter-cake was then collected and dried under vacuum to provide the title compound as a dark yellow solid (558 g, 90.5% purity, 85.4% yield). ¹H NMR (400 MHz, CDCl₃) δ 6.73 (m, 2H), 5.12 (s, 1H), 3.99 (s, 4H), 3.01 (m, 1H), 1.86 (m, 4H), 1.72 (m, 4H).

### Step 3: Preparation of 4-(3,5-difluoro-2-hydroxyphenyl)cyclohexan-1-one

The reaction vessel was charged with 2,4-difluoro-6-(1,4-dioxaspiro[4.5]decan-8-yl)phenol (100 g, 370 mmol) and methanol (1000 mL), then 6N hydrochloric acid (709 mL, 4.25 mol) was added and the reaction mixture was stirred at room temperature for 14 h under a nitrogen atmosphere. This procedure was carried out across five batches. The batches were then combined and concentrated to remove the methanol. The resulting suspension was filtered and the filter-cake was dried to provide crude product (819 g). The crude product was triturated with 20:1 petroleum ether/ethyl acetate (3 V) and the resulting mixture was stirred at 15° C. for 1 h. The mixture was then filtered and the filter-cake was dried under vacuum to provide 524 g of a yellow solid. This material was triturated with methyl tert-butyl ether (2.5 V) and the resulting mixture was stirred at 15° C. for 2 h. The mixture was then filtered, and the filter-cake was dried under vacuum to provide the title compound as a light yellow solid (385.5 g, 97.0% purity, 80.8% yield). ¹H NMR (400 MHz, CDCl₃) δ 6.71 (m, 2H), 5.38 (s, 1H), 3.47 (m, 1H), 2.54 (m, 4H), 2.23 (m, 2H), 1.89 (m, 2H).

### Step 4: Preparation of 4-(2-(benzyloxy)-3,5-difluorophenyl)cyclohexan-1-one

The reaction vessel was charged with 4-(3,5-difluoro-2-hydroxyphenyl)cyclohexan-1-one (142.5 g, 629.9 mmol) and acetonitrile (1.5 L), then potassium carbonate (174.1 g, 1.26 mol) and benzyl bromide (102.35 g, 598.4 mmol) were added while the reaction temperature was maintained at 15° C. The reaction mixture was then stirred at 25° C. for 12 h under a nitrogen atmosphere. This procedure was carried out across two batches. The two reactions were then combined and filtered, and the filtrate was concentrated. The crude product was triturated with 10:1 petroleum ether/ethyl acetate (2.5 V) and the resulting mixture was stirred at 15° C. for 1 h. The mixture was then filtered and the filter-cake was dried under vacuum to provide the title compound as an off-white solid (330.4 g, 98.2% purity, 82.9% yield). ¹H NMR (400 MHz, CDCl₃) δ 7.38 (m, 5H), 6.77 (m, 1H), 6.63 (m, 1H), 5.09 (s, 2H), 3.25 (t, J=12 Hz, 1H), 2.34 (m, 4H), 1.83 (m, 2H), 1.71 (m, 2H).

### Step 5: Preparation of (1s,4s)-4-(2-(benzyloxy)-3,5-difluorophenyl)cyclohexan-1-ol (Intermediate 3)

The reaction vessel was charged with triethylamine (83.2 g, 822 mmol) in dichloromethane (500 mL, 5V) and the solution was cooled to 0° C. Formic acid (34.9 g, 759 mmol) was then added, followed by chloro{[(1S,2S)-(+)-2-amino-1,2-diphenylethyl](4-toluenesulfonyl)amido}(p-cymene)ruthenium(II) (1.01 g, 1.58 mmol) and the solution was stirred at 0° C. for 30 minutes. A solution of 4-(2-(benzyloxy)-3,5-difluorophenyl)cyclohexan-1-one (100 g, 316 mmol) in dichloromethane (500 mL, 5V) was then added at 0° C. The reaction mixture was stirred at 10° C. for 1 h, then at 30° C. for 16 h. The reaction mixture was then concentrated under reduced pressure to remove the dichloromethane, after which water (10 V) was added to the residue followed by extraction with ethyl acetate (2×10 V). The combined organic layers were washed with brine (10 V), dried over sodium sulfate, filtered, and concentrated under reduced pressure. The crude product was purified by silica gel chromatography (40:1 petroleum ether/ethyl acetate) to provide the title compound initially as a yellow oil, which became a white solid upon cooling to 15° C. (80 g, 99.0% purity, 78.7% yield). ¹H NMR (400 MHz, CDCl₃) δ 7.37 (m, 5H), 7.14 (m, 1H), 6.85 (d, J=8.4 Hz, 1H), 4.98 (s, 2H), 4.33 (d, J=4 Hz, 1H), 3.84 (s, 1H), 1.79 (t, J=11.6 Hz), 1.68 (m, 4H), 1.42 (m, 2H), 1.21 (m, 2H).

### Synthesis of Intermediate 4: (2¹S,2⁴S,5²R,5³S)-5³-amino-1³,1⁵-difluoro-3,8-dioxa-5(2,1)-piperidina-1(1,2)-benzena-2(1,4)-cyclohexanacyclooctaphan-6-one

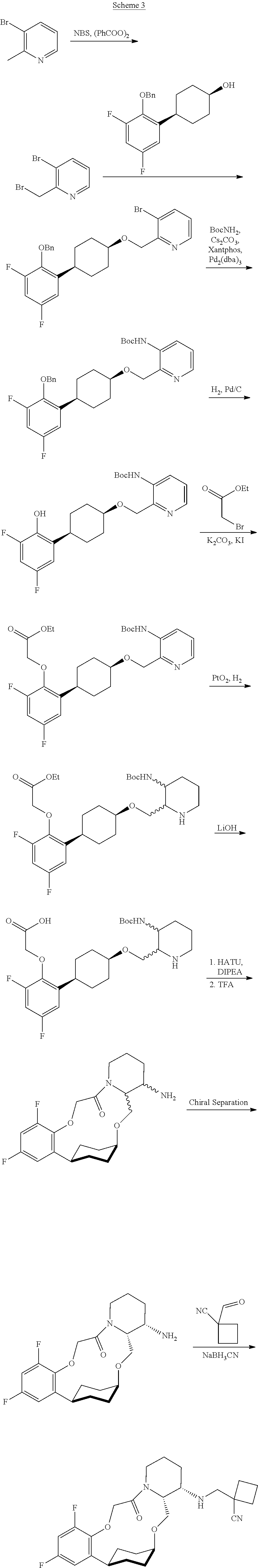

### Step 1: Preparation of 3-bromo-2-(bromomethyl)pyridine

A mixture of 2-methyl-3-bromopyridine (30.1 mL, 256 mmol), benzoyl peroxide (Luperox A98, 3.17 g, 12.8 mmol), and N-bromosuccinimide (50.2 g, 282 mmol) in carbon tetrachloride (427 mL) was degassed with nitrogen for 15 min then the solution was heated at reflux (85° C.) for 18 h. The reaction mixture was cooled to room temperature and filtered. The filter-cake was washed with 10% ethyl acetate/heptanes. The filtrate was concentrated and the resulting residue was purified by silica gel chromatography (0-15% ethyl acetate/heptanes) to provide the title compound as a purple oil which solidified upon storage at 4° C. LCMS (ESI): m/z [M+H]⁺ calcd for C₆H₅Br₂N: 253.87, found 253.9.

### Step 2: Preparation of 2-((((1s,4s)-4-(2-(benzyloxy)-3,5-difluorophenyl)cyclohexyl)oxy)methyl)-3-bromopyridine

Under nitrogen to a solution of (1s,4s)-4-(2-(benzyloxy)-3,5-difluorophenyl)cyclohexanol (Intermediate 3) (20.0 g, 62.8 mmol) in tetrahydrofuran (180 mL) was added sodium tert-butoxide (12.4 g, 126 mmol) and the solution was stirred at room temperature for 20 min. 3-bromo-2-(bromomethyl)pyridine (20.0 g, 79.7 mmol) in tetrahydrofuran (50 mL) was added slowly and the resulting solution was stirred at room temperature for 1 h. The reaction was quenched by addition of saturated ammonium chloride solution (100 mL) and extracted with ethyl acetate (200 mL). The organic layer was washed with brine (30 mL), dried over sodium sulfate, filtered, and concentrated. The crude product was purified by silica gel chromatography (0-30% ethyl acetate/heptanes gradient) to provide the title compound (25.0 g, 81% yield). LCMS (ESI): m/z [M+H]⁺ calcd for C₂₅H₂₄BrF₂NO₂: 490.09, found 490.0.

### Step 3: Preparation of tert-butyl (2-((((1s,4s)-4-(2-(benzyloxy)-3,5-difluorophenyl)cyclohexyl)oxy)methyl)pyridin-3-yl)carbamate

Under nitrogen, to a solution of 2-((((1s,4s)-4-(2-(benzyloxy)-3,5-difluorophenyl)cyclohexyl)oxy)methyl)-3-bromopyridine (8.98 g, 18.4 mmol) in 1,4-dioxane (92 mL) were added cesium carbonate (8.39 g, 25.7 mmol) and tert-butyl carbamate (3.30 g, 27.6 mmol). The solution was sparged with nitrogen, 4,4-bis(diphenylphosphino)-9,9-dimethylxanthene (1.09 g, 1.84 mmol) and tris(dibenzylideneacetone)-dipalladium(0) (859 mg, 0.92 mmol) were added. Sparking was continued for 5 min then the solution was stirred under a nitrogen atmosphere at 100° C. for 17 h. The reaction was diluted with ethyl acetate (200 mL) and brine (100 mL). The mixture was filtered and the organic layer was washed with brine, dried over sodium sulfate, filtered, and concentrated. The crude product was purified by silica gel chromatography (0-70% ethyl acetate/heptanes gradient) to provide the title compound (9.36 g, 97% yield). LCMS (ESI): m/z [M+H]⁺ calcd for C₃₀H₃₄F₂N₂O₄: 525.25, found 525.3.

### Step 4: Preparation of tert-butyl (2-((((1s,4s)-4-(3,5-difluoro-2-hydroxyphenyl)cyclohexyl)oxy)methyl)pyridin-3-yl)carbamate

A solution of tert-butyl (2-((((1s,4s)-4-(2-(benzyloxy)-3,5-difluorophenyl) cyclohexyl)oxy)methyl)pyridin-3-yl)carbamate (9.36 g, 17.8 mmol) in ethanol (89.2 mL) was purged with nitrogen for 5 min, then palladium on carbon (10 wt. % loading, 936 mg, 8.80 mmol) was added. The reaction vessel was evacuated and filled with hydrogen five times, then the reaction was stirred under hydrogen (1 atm) for 18 h. The hydrogen was removed through four cycles of evacuation and nitrogen fills, then the suspension was filtered over Celite, washed with ethyl acetate, dichloromethane, and ethanol, then concentrated to provide the title compound as a foam (7.55 g, 97% yield). LCMS (ESI): m/z [M+H]⁺ calcd for C₂₃H₂₈F₂N₂O₄: 435.20, found 435.2.

### Step 5: Preparation of ethyl 2-(2-((1s,4s)-4-((3-((tert-butoxycarbonyl)amino)pyridin-2-yl)methoxy)cyclohexyl)-4,6-difluorophenoxy)acetate

To tert-butyl (2-((((1s,4s)-4-(3,5-difluoro-2-hydroxyphenyl)cyclohexyl) oxy)methyl)pyridin-3-yl)carbamate (7.55 g, 17.4 mmol) in acetone (86.9 mL) were added potassium carbonate (7.19 g, 52.1 mmol) and potassium iodide (147 mg, 869 μmol) followed by dropwise addition of ethyl bromoacetate (2.56 mL, 22.6 mmol). The solution was stirred at 50° C. for 2 h. The reaction was cooled to room temperature then the mixture was filtered and washed with acetone. The filtrate was then concentrated, and the crude product was purified by silica gel chromatography (0-80% ethyl acetate/heptanes gradient, loaded in toluene) to provide the title compound as a brown oil (7.64 g, 84% yield). LCMS (ESI): m/z [M+H]⁺ calcd for C₂₇H₃₄F₂N₂O₆: 521.24, found 521.3.

### Step 6: Preparation of ethyl 2-(2-((1s,4s)-4-((3-((tert-butoxycarbonyl)amino)piperidin-2-yl)methoxy)cyclohexyl)-4,6-difluorophenoxy)acetate

To a solution of ethyl 2-(2-((1s,4s)-4-((3-((tert-butoxycarbonyl)amino)pyridin-2-yl)methoxy)cyclohexyl)-4,6-difluorophenoxy)acetate (7.64 g, 14.7 mmol) in a mixture of ethanol (110 mL) and acetic acid (12.2 mL) under nitrogen was added platinum (IV) oxide (833 mg, 3.67 mmol). The reaction was evacuated and backfilled with hydrogen, then the reaction mixture was stirred at room temperature under a hydrogen atmosphere for 16 h. Additional platinum (IV) oxide (183 mg, 766 μmol) was added and the reaction was stirred under a hydrogen atmosphere for an additional 5 h. The hydrogen atmosphere was then evacuated and backfilled with nitrogen, after which the reaction mixture was filtered through Celite, rinsed with ethyl acetate, dichloromethane, and ethanol. The filtrated was then concentrated and the crude product was dissolved in dichloromethane and extracted with saturated sodium bicarbonate solution. The aqueous layer was extracted twice more with dichloromethane, and the combined organic layers were dried over sodium sulfate, filtered, and concentrated. The crude product was purified by silica gel chromatography (0-10% methanol/dichloromethane gradient) to provide the title compound as a pale grey gum isolated as a mixture of cis enantiomers (4.14 g, 54% yield). LCMS (ESI): m/z [M+H]⁺ calcd for C₂₇H₄₀F₂N₂O₆: 527.29, found 526.9.

### Step 7: Preparation of 2-(2-((1s,4s)-4-((3-((tert-butoxycarbonyl)amino)piperidin-2-yl)methoxy)cyclohexyl)-4,6-difluorophenoxy)acetic acid

To a solution of ethyl 2-(2-((1s,4s)-4-((3-((tert-butoxycarbonyl)amino)piperidin-2-yl)methoxy)cyclohexyl)-4,6-difluorophenoxy)acetate (3.34 g, 6.34 mmol) in a mixture of tetrahydrofuran (21.4 mL) and ethanol (10.7 mL) was added a 1 M aqueous solution of lithium hydroxide (6.98 mL, 6.98 mmol) and the resulting mixture was stirred at room temperature for 2 h. The reaction was quenched by addition of 1 M aqueous hydrochloride acid (634 μL, 634 mol), then the mixture was concentrated. Acetonitrile was added and the mixture was again concentrated. Water and acetonitrile were added and the mixture was frozen and lyophilized to provide the title compound as a white solid, which was used without further purification (3.25 g, 101% yield). LCMS (ESI): m/z [M+H]⁺ calcd for C₂₅H₃₆F₂N₂O₆: 499.25, found 499.2.

### Step 8: Preparation of (2¹s,2⁴s)-5³-amino-1³,1⁵-difluoro-3,8-dioxa-5(2,1)-piperidina-1(1,2)-benzena-2(1,4)-cyclohexanacyclooctaphan-6-one

To a solution of 2-(2-((1s,4s)-4-((3-((tert-butoxycarbonyl)amino)piperidin-2-yl)methoxy)cyclohexyl)-4,6-difluorophenoxy)acetic acid (800 mg, 1.60 mmol) in dichloroethane (730 mL) at an internal temperature of 70° C. was added N,N-diisopropylethylamine (1.12 mL, 6.42 mmol) followed by HATU (934 mg, 2.41 mmol) in one portion. The solution was stirred at 80° C. for 17 min. The solution was concentrated to half of the volume and poured into a saturated aqueous solution of sodium bicarbonate. The organic phase was separated, and the aqueous phase was extracted with dichloromethane (2×100 mL). The combined organic layers were dried over sodium sulfate, filtered, and concentrated to provide the crude intermediate. This intermediate (788 mg, 1.64 mmol) in was dissolved in dichloromethane (13.5 mL) and trifluoroacetic acid (4.32 mL, 56.4 mmol) was added. The mixture was then stirred at room temperature for 15 min. The mixture was concentrated to dryness, after which toluene (20 mL) was added, and the solution was again concentrated. The crude product was purified by silica gel chromatography (0-10% methanol/dichloromethane gradient with 1% ammonium hydroxide) to provide the title compound as a white solid (240 mg, 38% yield). LCMS (ESI): m/z [M+H]⁺ calcd for C₂₀H₂₆F₂N₂O₃: 381.19, found 381.1.

### Step 9: Preparation of (2¹S,2⁴S,5²R,5³S)-5³-amino-1³,1⁵-difluoro-3,8-dioxa-5(2,1)-piperidina-1(1,2)-benzena-2(1,4)-cyclohexanacyclooctaphan-6-one (Intermediate 4)

Racemic (21s,2⁴s)-5³-amino-1³,1⁵-difluoro-3,8-dioxa-5(2,1)-piperidina-1(1,2)-benzena-2(1,4)-cyclohexanacyclooctaphan-6-one was purified by chiral chromatography (ChiralPak IA, 250 mm×4.6 mm ID, 5 μm; 20:20:60 methanol/ethanol/hexanes with 0.1% diethylamine; 0.8 mL/min, column temperature: 22° C., 10 minute run time) to provide the title compound.

### Preparation of Compound 21: 1-((((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)methyl)cyclobutane-1-carbonitrile

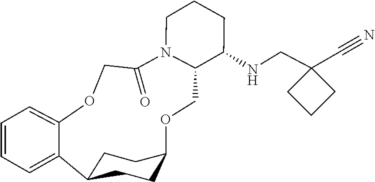 [SMILES: N#CC1(CN[C@H]2CCCN3C(=O)COc4ccccc4[C@H]4CC[C@H](CC4)OC[C@@H]23)CCC1]

To a solution of Intermediate 2 (40.0 mg, 116 μmol) and 1-formylcyclobutanecarbonitrile (25.3 mg, 232 μmol) in dichloromethane (0.8 mL) were added acetic acid (80 μL) and molecular sieves, then the reaction was stirred at room temperature for 18 h. Sodium cyanoborohydride (38.4 mg, 581 μmol) was added and the reaction was stirred at room temperature for 1 h. The reaction was diluted with water (10 mL) and extracted with dichloromethane (3×10 mL). The combined organic phases were dried over magnesium sulfate, filtered, and concentrated. The crude product was purified by preparative HPLC (C18 column, 40-60% acetonitrile/water gradient with 10 mM ammonium formate) to provide the title compound as a white solid (25 mg, 49% yield). LCMS (ESI): m/z [M+H]⁺ calcd for C₂₆H₃₅N₃O₃: 438.27, found 438.3. ¹H NMR (400 MHz, Methanol-d₄) δ 7.14 (td, J=7.8, 1.7 Hz, 1H), 7.06 (dd, J=7.4, 1.6 Hz, 1H), 6.87-6.81 (m, 2H), 5.29 (d, J=10.6 Hz, 1H), 5.25-5.17 (m, 1H), 4.10 (d, J=10.6 Hz, 1H), 3.92 (dd, J=11.0, 9.5 Hz, 1H), 3.79 (d, J=12.9 Hz, 1H), 3.68 (s, 1H), 3.51 (ddd, J=15.9, 11.2, 3.4 Hz, 2H), 3.02 (d, J=6.3 Hz, 2H), 2.94-2.87 (m, 1H), 2.74 (ddd, J=25.6, 12.9, 4.5 Hz, 1H), 2.56 (ddd, J=17.5, 12.1, 5.3 Hz, 1H), 2.51-2.40 (m, 2H), 2.37-2.27 (m, 1H), 2.14 (ddddd, J=25.2, 18.3, 9.2, 4.9, 2.5 Hz, 5H), 1.90-1.76 (m, 3H), 1.73-1.22 (m, 7H).

### Synthesis of Compound 22: 4-((((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)methyl)tetrahydro-2H-pyran-4-carbonitrile

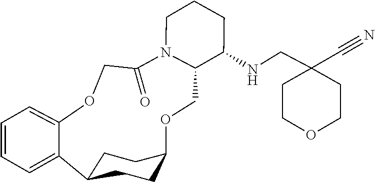 [SMILES: N#CC1(CN[C@H]2CCCN3C(=O)COc4ccccc4[C@H]4CC[C@H](CC4)OC[C@@H]23)CCOCC1]

### Step 1: Preparation of 1-formylcyclohexanecarbonitrile

4-(hydroxymethyl)tetrahydro-2H-pyran-4-carbonitrile (99.9 mg, 708 mol) was dissolved in dichloromethane (3.5 mL) and Dess-Martin periodinane (360 mg, 849 μmol) was added. The resulting solution was stirred at room temperature for 18 h. To the reaction was added saturated aqueous sodium carbonate (5 mL) and 1 M aqueous sodium thiosulfate (2 mL), and the mixture was stirred for 1 h. The aqueous phase was extracted with dichloromethane (2×10 mL). The combined organic phases were dried over magnesium sulfate, filtered, and concentrated to provide the title compound as a white solid, which was used without further purification.

### Step 2: Preparation of 4-((((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)methyl)tetrahydro-2H-pyran-4-carbonitrile (Compound 22)

To a solution of Intermediate 2 (40.0 mg, 116 μmol) in dichloromethane (0.53 mL) and acetic acid (52.8 μL) were added 1-formylcyclohexanecarbonitrile (17.8 mg, 128 mol) and molecular sieves, then the reaction was stirred at room temperature for 18 h. Sodium cyanoborohydride (38.4 mg, 581 μmol) was added and the reaction was stirred at room temperature for 1 h. The reaction was diluted with water (10 mL) and extracted with dichloromethane (3×10 mL). The combined organic phases were dried over magnesium sulfate, filtered, and concentrated. The crude product was purified by preparative HPLC (C18 column, 40-60% acetonitrile/water gradient with 10 mM ammonium formate) to provide the title compound as a white solid (22.1 mg, 41% yield). LCMS (ESI): m/z [M+H]⁺ calcd for C₂₇H₃₇N₃O₄: 468.28, found 468.3. ¹H NMR (400 MHz, Methanol-d₄) δ 7.14 (td, J=7.8, 1.7 Hz, 1H), 7.06 (dd, J=7.4, 1.6 Hz, 1H), 6.88-6.81 (m, 2H), 5.29 (d, J=10.6 Hz, 1H), 5.23-5.15 (m, 1H), 4.10 (d, J=10.6 Hz, 1H), 4.00-3.88 (m, 3H), 3.78 (d, J=14.9 Hz, 1H), 3.71-3.60 (m, 3H), 3.56-3.44 (m, 2H), 2.92-2.83 (m, 3H), 2.73 (qd, J=12.7, 4.4 Hz, 1H), 2.61-2.51 (m, 1H), 2.31 (ddd, J=25.9, 13.0, 4.2 Hz, 1H), 2.16 (d, J=14.2 Hz, 1H), 1.94-1.78 (m, 5H), 1.73-1.22 (m, 9H).

### Synthesis of Compound 23: 2-(1-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)cyclobutyl)acetonitrile

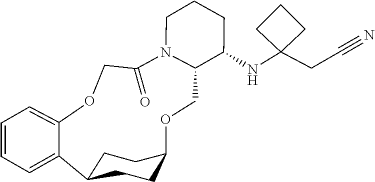 [SMILES: N#CCC1(N[C@H]2CCCN3C(=O)COc4ccccc4[C@H]4CC[C@H](CC4)OC[C@@H]23)CCC1]

### Step 1: Preparation of 2-cyclobutylideneacetonitrile

Lithium bromide (1.96 g, 22.5 mmol) was flame dried under vacuum and cooled to room temperature under nitrogen. Tetrahydrofuran (40 mL) was added and the mixture was stirred until homogeneous. Diethyl cyanomethylphosphonate (4.1 mL, 24.1 mmol) was added followed by triethylamine (3.2 mL, 22.5 mmol). The reaction was stirred at 25° C. for 2 h and then was added to a solution of cyclobutanone (1.74 mL, 22.5 mmol) in tetrahydrofuran (5.0 mL). The resulting solution was stirred for 20 h at room temperature. The mixture was concentrated with silica and was dry-loaded and purified by silica gel chromatography (0-15% ethyl acetate/heptanes gradient) to provide the title compound as a colorless oil (581 mg, 28% yield). ¹H NMR (400 MHz, CDCl₃) δ 5.12-5.08 (m, 1H), 3.00-2.91 (m, 2H), 2.90-2.81 (m, 2H), 2.09 (dq, J=11.6, 7.9 Hz, 2H).

### Step 2: Preparation of 2-(1-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)cyclobutyl)acetonitrile (Compound 23)

In a microwave tube, Intermediate 2 (50.0 mg, 145 mol) was dissolved in acetonitrile (0.73 mL), then 1,8-diazabicyclo[5.4.0]undec-7-ene (DBU) (11.1 μL, 72.6 μmol) and 2-cyclobutylideneacetonitrile (27.0 mg, 290 μmol) were added. The solution was stirred at 110° C. for 4 days and then at 130° C. for 4 days. The reaction mixture was directly loaded for purification by preparative HPLC (C18 column, 45-65% acetonitrile/water gradient with 10 mM ammonium formate) to provide the title compound as a beige solid (24.4 mg, 38% yield). LCMS (ESI): m/z [M+H]⁺ calcd for C₂₆H₃₅N₃O₃: 438.27, found 438.3. ¹H NMR (400 MHz, Methanol-d₄) δ 7.14 (td, J=7.7, 1.7 Hz, 1H), 7.06 (dd, J=7.4, 1.6 Hz, 1H), 6.88-6.80 (m, 2H), 5.28 (d, J=10.6 Hz, 1H), 5.00-4.93 (m, 1H), 4.10 (d, J=10.6 Hz, 1H), 3.96-3.89 (m, 1H), 3.75 (d, J=13.6 Hz, 1H), 3.70 (dd, J=9.0, 3.8 Hz, 2H), 3.52-3.43 (m, 1H), 2.88 (dt, J=10.2, 5.0 Hz, 1H), 2.84 (d, J=1.7 Hz, 2H), 2.78-2.68 (m, 1H), 2.56 (ddd, J=17.5, 12.1, 5.2 Hz, 1H), 2.31 (ddd, J=25.8, 13.0, 4.1 Hz, 1H), 2.12 (ddd, J=18.3, 15.2, 10.4 Hz, 3H), 2.01 (ddd, J=16.7, 8.2, 4.3 Hz, 2H), 1.89-1.75 (m, 5H), 1.67-1.55 (m, 2H), 1.50-1.39 (m, 2H), 1.34 (dt, J=13.9, 7.0 Hz, 1H), 1.26 (dd, J=10.7, 5.5 Hz, 1H).

### Preparation of Compound 24: 3-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)pyridazine-4-carbonitrile

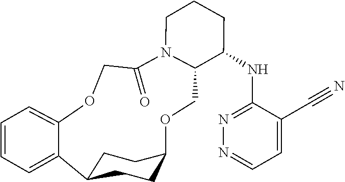 [SMILES: N#Cc1ccnnc1N[C@H]1CCCN2C(=O)COc3ccccc3[C@H]3CC[C@H](CC3)OC[C@@H]12]

A solution of Intermediate 2 (40.0 mg, 116 μmol), triethylamine (98.1 μL, 697 μmol) and 3-chloropyridazine-4-carbonitrile (21.7 mg, 151 μmol) in N-methyl-2-pyrrolidone (0.58 mL) was heated at 100° C. for 18 h. The reaction was cooled to room temperature, diluted with ethyl acetate, washed with 1 M hydrochloric acid, saturated aqueous sodium bicarbonate solution, and brine. The organic phase was dried over sodium sulfate, filtered, and concentrated. The crude product was purified by preparative HPLC (C18 column, 20-80% acetonitrile/water gradient with 10 mM ammonium formate) to provide the title compound as a mauve solid (22.9 mg, 44% yield). LCMS (ESI): m/z [M+H]⁺ calcd for C₂₅H₂₉N₅O₃: 448.23, found 448.2. ¹H NMR (400 MHz, Methanol-d₄) δ 8.66 (d, J=4.9 Hz, 1H), 7.71 (d, J=4.8 Hz, 1H), 7.14 (td, J=7.8, 1.7 Hz, 1H), 7.08-7.04 (m, 1H), 6.88-6.83 (m, 2H), 5.62-5.51 (m, 1H), 5.34 (d, J=10.5 Hz, 1H), 4.67-4.54 (m, 1H), 4.11 (d, J=10.5 Hz, 1H), 4.06 (dd, J=10.8, 9.3 Hz, 1H), 3.87 (d, J=13.3 Hz, 1H), 3.67 (s, 1H), 3.63-3.52 (m, 1H), 3.39 (dd, J=9.1, 3.9 Hz, 1H), 2.81-2.69 (m, 1H), 2.61-2.50 (m, 1H), 2.33-2.14 (m, 2H), 2.07-2.00 (m, 1H), 2.00-1.90 (m, 1H), 1.84-1.69 (m, 2H), 1.49-1.19 (m, 5H).

### Preparation of Compound 25: 3-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)pyrazine-2-carbonitrile

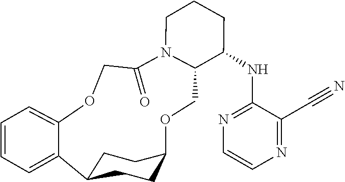 [SMILES: N#Cc1nccnc1N[C@H]1CCCN2C(=O)COc3ccccc3[C@H]3CC[C@H](CC3)OC[C@@H]12]

A solution of Intermediate 2 (40.0 mg, 116 mol), triethylamine (98.1 μL, 697 μmol) and 2-chloro-3-cyanopyrazine (21.7 mg, 151 mol) in N-methyl-2-pyrrolidone (0.58 mL) was heated at 65° C. for 72 h. The reaction was cooled to room temperature, diluted with ethyl acetate, washed with 1 M hydrochloric acid, followed by saturated aqueous sodium bicarbonate solution and brine. The organic phase was dried over sodium sulfate, filtered, and concentrated. The crude product was purified by preparative HPLC (C18 column, 25-60% acetonitrile/water gradient with 10 mM ammonium formate) to provide the title compound (31 mg, 60% yield). LCMS (ESI): m/z [M+H]⁺ calcd for C₂₅H₂₉N₅O₃: 448.23, found 448.2. ¹H NMR (400 MHz, Methanol-d₄) δ 8.34 (d, J=2.4 Hz, 1H), 7.92 (d, J=2.4 Hz, 1H), 7.14 (td, J=7.7, 1.7 Hz, 1H), 7.06 (dd, J=7.3, 1.6 Hz, 1H), 6.86 (t, J=7.5 Hz, 2H), 5.52-5.42 (m, 1H), 5.33 (d, J=10.5 Hz, 1H), 4.38 (dt, J=12.5, 5.0 Hz, 1H), 4.11 (d, J=10.6 Hz, 1H), 4.03 (dd, J=11.0, 9.3 Hz, 1H), 3.85 (d, J=13.6 Hz, 1H), 3.66 (s, 1H), 3.56 (td, J=13.4, 2.6 Hz, 1H), 3.35 (dd, J=9.1, 3.9 Hz, 1H), 2.84-2.63 (m, 1H), 2.63-2.41 (m, 1H), 2.33-2.13 (m, 2H), 2.08-1.86 (m, 3H), 1.81-1.64 (m, 2H), 1.50-1.20 (m, 4H).

### Preparation of Compound 26: 2-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)-6-(trifluoromethyl)nicotinonitrile

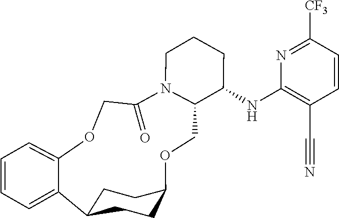 [SMILES: N#Cc1ccc(C(F)(F)F)nc1N[C@H]1CCCN2C(=O)COc3ccccc3[C@H]3CC[C@H](CC3)OC[C@@H]12]

A solution of Intermediate 2 (40.0 mg, 116 μmol), N,N-diisopropylethylamine (40.9 μL, 232 μmol) and 2-chloro-6-(trifluoromethyl)nicotinonitrile (49.0 mg, 232 μmol) in N-methyl-2-pyrrolidone (0.3 mL) was stirred at 130° C. for 4 h. The crude reaction mixture was then loaded directly and purified by preparative HPLC (C18 column, 35-100% acetonitrile/water gradient with 10 mM ammonium formate) to provide the title compound as a beige solid (48.8 mg, 82% yield). LCMS (ESI): m/z [M+H]⁺ calcd for C₂₇H₂₉F₃N₄O₃: 515.22, found 515.3. ¹H NMR (400 MHz, DMSO-d₆) δ 8.25 (d, J=7.8 Hz, 1H), 7.50 (d, J=7.3 Hz, 1H), 7.20-7.12 (m, 2H), 7.11-6.97 (m, 1H), 6.91-6.73 (m, 2H), 5.37-5.18 (m, 2H), 4.21 (d, J=4.9 Hz, 1H), 3.97-3.86 (m, 2H), 3.71 (d, J=11.8 Hz, 1H), 3.61 (s, 1H), 3.24 (dd, J=9.3, 3.7 Hz, 1H), 2.73-2.62 (m, 1H), 2.27-1.95 (m, 3H), 1.93-1.48 (m, 5H), 1.45-1.04 (m, 5H).

### Preparation of Compound 27: 1-((((2¹S,2⁴S,5²R,5³S)-13,15-difluoro-6-oxo-3,8-dioxa-5(2,1)-piperidina-1(1,2)-benzena-2(1,4)-cyclohexanacyclooctaphane-5³-yl)amino)methyl)cyclopropane-1-carbonitrile

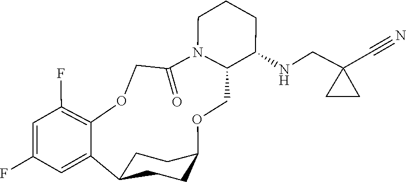 [SMILES: N#CC1(CN[C@H]2CCCN3C(=O)COc4c(F)cc(F)cc4[C@H]4CC[C@H](CC4)OC[C@@H]23)CC1]

To a solution of (2¹s,2⁴s)-5³-amino-1³,1⁵-difluoro-3,8-dioxa-5(2,1)-piperidina-1(1,2)-benzena-2(1,4)-cyclohexanacyclooctaphan-6-one (50 mg, 0.131 mmol) and 1-formylcyclopropane-1-carbonitrile (18.8 mg, 0.197 mmol) in tetrahydrofuran (3 mL) was added sodium borohydride (15 mg, 0.349 mmol) at 0° C. The reaction was allowed to warm to 20° C. and stirred for an additional 10 hours, then was quenched with water (5 mL) and extracted with ethyl acetate (5 mL×3). The combined organic extracts were washed with water, brine, dried over sodium sulfate, filtered, and concentrated. The crude product was purified by preparative HPLC (XBridge C18 OBD Column 250 mm×19 mm×5 m, 55-80% acetonitrile/water gradient with 10 mM ammonium formate) to provide the racemate of the title compound as a white solid (10 mg, 16.26% yield). The mixture of enantiomers was then separated by chiral HPLC (CHIRALPAK IF, 2*25 cm, 5 m; Mobile Phase A: hexanes with 0.5% of 2M ammonia in methanol, Mobile Phase B: ethanol; Flow rate: 13 mL/min; 50% isocratic method. The title compound was isolated as the later eluting peak (4.1 mg, 41% yield). LCMS (ESI): m/z [M+H]⁺ calcd for C₂₅H₃₁F₂N₃O₃: 460.24, found 460.25. ¹H NMR (300 MHz, Acetonitrile-d₃) δ 6.80-6.94 (m, 1H), 6.70-6.80 (m, 1H), 5.39 (dd, J=12.2, 3.4 Hz, 1H), 5.17-5.04 (m, 1H), 4.13 (dd, J=12.2, 1.9 Hz, 1H), 3.76-3.89 (m, 2H), 3.70 (s, 1H), 3.35-3.61 (m, 2H), 2.77-2.91 (m, 2H), 2.71-2.77 (m, 1H), 2.47-2.70 (m, 2H), 2.08-2.16 (m, 1H), 2.00-2.08 (m, 1H), 1.68-1.85 (m, 3H), 1.29-1.65 (m, 7H), 1.17-1.22 (m, 2H), 1.10-1.17 (m, 1H), 0.88-1.02 (m, 2H). ¹⁹F NMR (282 MHz, Acetonitrile-d₃) δ −119.23, 127.68.

### Synthesis of Compound 28: 1-((((2¹S,2⁴S,5²R,5³S)-1³,1⁵-difluoro-6-oxo-3,8-dioxa-5(2,1)-piperidina-1(1,2)-benzena-2(1,4)-cyclohexanacyclooctaphane-5³-yl)amino)methyl)cyclobutane-1-carbonitrile

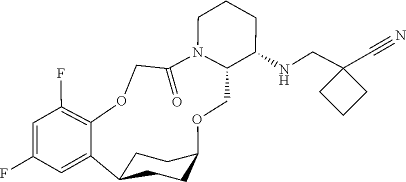 [SMILES: N#CC1(CN[C@H]2CCCN3C(=O)COc4c(F)cc(F)cc4[C@H]4CC[C@H](CC4)OC[C@@H]23)CCC1]

### Step 1: Preparation of 1-(hydroxymethyl)cyclobutane-1-carbonitrile

To a mixture of ethyl 1-cyanocyclobutanecarboxylate (246 mg, 1.53 mmol) in tetrahydrofuran (3.9 mL) and methanol (0.98 mL) at 0° C. was added sodium borohydride (121 mg, 3.08 mmol) and the mixture was slowly warmed to room temperature and stirred for 1 h. Water (20 mL) was added and the mixture was extracted with dichloromethane (3×15 mL). The combined organic layers were dried over sodium sulfate, filtered, and concentrated to provide the title compound as a colorless oil which was used without further purification (quantitative yield was assumed).

### Step 2: Preparation of 1-formylcyclobutane-1-carbonitrile

1-(hydroxymethyl)cyclobutane-1-carbonitrile was dissolved in dichloromethane (7.5 mL) and Dess-Martin periodinane (772 mg, 1.82 mmol) was added. The reaction was stirred at room temperature for 21 h. To the reaction was added saturated sodium bicarbonate solution (10 mL) and 10% sodium thiosulfate solution (10 mL) and the mixture was stirred until a clear solution was obtained (1 h). The aqueous phase was extracted with dichloromethane (2×20 mL) and the combined organic layers were dried over sodium sulfate, filtered, and concentrated at to provide the title compound as a yellow oil, which was used without further purification.

### Step 3: Preparation of 1-((((2¹S,2⁴S,5²R,5³S)-1³,1⁵-difluoro-6-oxo-3,8-dioxa-5(2,1)-piperidina-1(1,2)-benzena-2(1,4)-cyclohexanacyclooctaphane-5³-yl)amino)methyl)cyclobutane-1-carbonitrile (Compound 28)

To a solution of Intermediate 4 (100 mg, 263 μmol) and 1-formylcyclobutanecarbonitrile (57.4 mg, 526 μmol) in dichloromethane (1.8 mL) were added acetic acid (181 μL) and molecular sieves. The reaction was stirred at room temperature for 2 h, then sodium cyanoborohydride (86.9 mg, 1.31 mmol) was added and the reaction was stirred for an additional 1 h. The reaction was diluted with water (30 mL) and extracted with dichloromethane (3×25 mL). The combined organic layers were dried over sodium sulfate, filtered, and concentrated. The crude product was purified by preparative HPLC (C18 column, 30-100% acetonitrile/water gradient with 10 mM ammonium formate) to provide the title compound as a white solid (101 mg, 81% yield). LCMS (ESI): m/z [M+H]⁺ calcd for C₂₆H₃₃F₂N₃O₃: 474.25, found 474.2. ¹H NMR (400 MHz, Methanol-d₄) δ 6.86 (ddd, J=11.5, 8.3, 3.1 Hz, 1H), 6.74 (ddd, J=9.1, 3.0, 1.9 Hz, 1H), 5.47 (dd, J=12.4, 3.2 Hz, 1H), 5.26-5.16 (m, 1H), 4.14 (dd, J=12.4, 1.8 Hz, 1H), 3.95-3.82 (m, 2H), 3.71 (s, 1H), 3.58-3.44 (m, 2H), 3.03 (s, 2H), 2.94-2.83 (m, 1H), 2.77-2.55 (m, 2H), 2.52-2.39 (m, 2H), 2.30-2.00 (m, 6H), 1.94-1.78 (m, 3H), 1.72-1.27 (m, 6H).

### Synthesis of Compound 29: 3-((((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)methyl)oxetane-3-carbonitrile

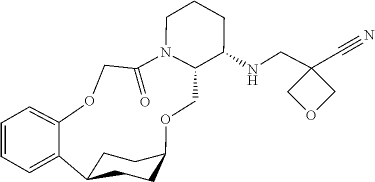 [SMILES: N#CC1(CN[C@H]2CCCN3C(=O)COc4ccccc4[C@H]4CC[C@H](CC4)OC[C@@H]23)COC1]

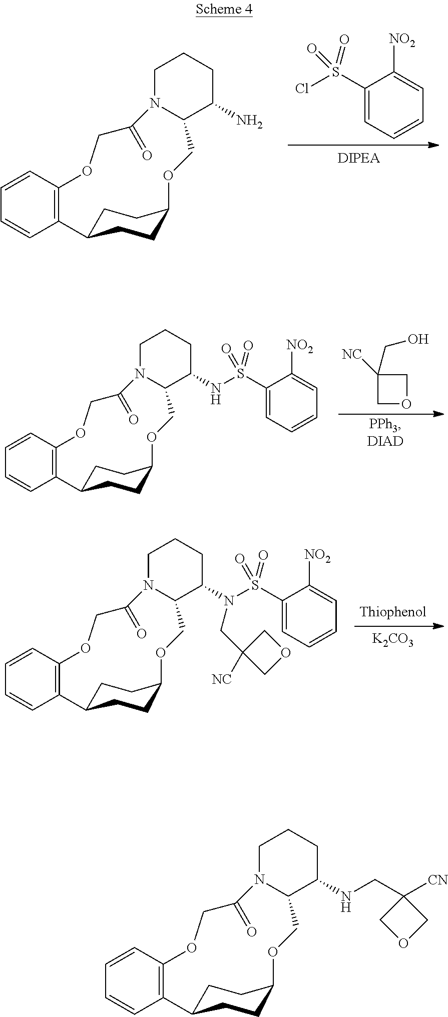

### Step 1: Preparation of N-(2¹S,2⁴S,5²R,5³S)-6-oxo-3,8-dioaxa-5(2,1)piperidina-1(1,2)-benzena-2(1,4)cyclohexanaoctaphane-5³-yl)-2-nitrobenzenesulfonamide

To a solution of Intermediate 2 (120 mg, 348 mol) in dichloromethane (4.8 mL) was added N,N-diisopropylethylamine (121 μL, 697 μmol) followed by 2-nitrobenzenesulfonyl chloride (78.8 mg, 348 mol). The reaction was stirred at room temperature for 1 h, concentrated, and the crude product was purified by preparative HPLC (C18 column, 40-60% acetonitrile/water gradient with 10 mM ammonium formate) to provide the title compound as a white solid (160 mg, 87% yield). LCMS (ESI): m/z [M+H]⁺ calcd for C₂₆H₃₁N₃O₇S: 530.19, found 530.2.

### Step 2: Preparation of N-(2¹S,2⁴S,5²R,5³S)-6-oxo-3,8-dioaxa-5(2,1)piperidina-1(1,2)-benzena-2(1,4)cyclohexanaoctaphane-5³-yl)-(N-((3-cyanooxetan-3-yl)methyl)-2-nitrobenzenesulfonamide

N-(2¹S,2⁴S,5²R,5³S)-6-oxo-3,8-dioaxa-5(2,1)piperidina-1(1,2)-benzena-2(1,4)cyclohexanaoctaphane-5³-yl)-2-nitrobenzenesulfonamide (136 mg, 256 μmol) was dissolved in toluene (0.13 mL) and to the solution were added 3-(hydroxymethyl)oxetane-3-carbonitrile (64.2 μL, 563 μmol) and triphenylphosphine (5.50 mg, 20.8 mol). The reaction was heated to 60° C., diisopropyl azodicarboxylate (113 μL, 563 μmol) was added, and the reactions was stirred at 60° C. for 20 h. The reaction mixture was concentrated and the crude product was purified by preparative HPLC (C18 column, 40-60% acetonitrile/water gradient with 10 mM ammonium formate) to provide the title compound (45 mg, 28% yield). LCMS (ESI): m/z [M+H]⁺ calcd for C₃₁H₃₆N₄O₈S: 625.23, found 625.2.

### Step 3: Preparation of 3-((((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)methyl)oxetane-3-carbonitrile (Compound 29)

To a solution of N-(2'S,2⁴S,5²R,5³S)-6-oxo-3,8-dioaxa-5(2,1)piperidina-1(1,2)-benzena-2(1,4)cyclohexanaoctaphane-5³-yl)-(N-((3-cyanooxetan-3-yl)methyl)-2-nitrobenzenesulfonamide (75.0 mg, 120 μmol) in acetonitrile (1.0 mL) was added potassium carbonate (49.8 mg, 360 μmol) followed by thiophenol (39.1 μL, 372 μmol). The reaction was stirred at room temperature for 18 h and was then diluted with 2M sodium hydroxide and extracted with dichloromethane (3×10 mL). The combined organic phases were dried over magnesium sulfate, filtered, and concentrated. The crude produce was purified by preparative HPLC (C18 column, 35-55% acetonitrile/water gradient with 10 mM ammonium formate) to provide the title compound as an off-white solid (5.7 mg, 11% yield). LCMS (ESI): m/z [M+H]⁺ calcd for C₂₅H₃₃N₃O₄: 440.25, found 440.3. ¹H NMR (400 MHz, Methanol-d₄) δ 7.14 (td, J=7.8, 1.7 Hz, 1H), 7.06 (dd, J=7.4, 1.6 Hz, 1H), 6.89-6.81 (m, 2H), 5.29 (d, J=10.6 Hz, 1H), 5.25-5.17 (m, 1H), 4.86 (dd, J=6.4, 3.0 Hz, 2H), 4.60 (dd, J=8.3, 6.4 Hz, 2H), 4.10 (d, J=10.6 Hz, 1H), 3.93 (dd, J=10.9, 9.6 Hz, 1H), 3.79 (d, J=15.0 Hz, 1H), 3.68 (s, 1H), 3.54-3.44 (m, 2H), 2.97-2.87 (m, 1H), 2.73 (qd, J=12.9, 4.4 Hz, 1H), 2.61-2.50 (m, 1H), 2.31 (ddd, J=26.0, 13.1, 4.2 Hz, 1H), 2.16 (d, J=14.0 Hz, 1H), 1.84 (ddd, J=19.5, 15.2, 4.8 Hz, 3H), 1.71-1.20 (m, 8H).

### Preparation of Compound 30: 3-(((2¹S,2⁴S,5²R,5³S)-6-oxo-3,8-dioxa-5(2,1)-piperidina-1(1,2)-benzena-2(1,4)-cyclohexanacyclooctaphane-5³-yl)amino)pyrazine-2-carbonitrile

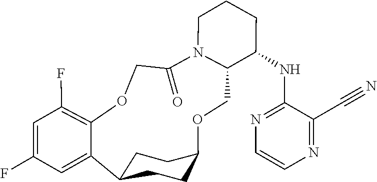 [SMILES: N#Cc1nccnc1N[C@H]1CCCN2C(=O)COc3c(F)cc(F)cc3[C@H]3CC[C@H](CC3)OC[C@@H]12]

To a solution of Intermediate 4 (70.0 mg. 184 μmol) in N-methyl-2-pyrrolidone (0.92 mL) were added 2-chloro-3-cyanopyrazine (34.1 mg, 239 μmol) and triethylamine (155 μL, 1.10 mmol). The reaction mixture was stirred at 85° C. for 18 h. The crude reaction mixture was directly loaded and purified by preparative HPLC (C18 column, 30-100% acetonitrile/water gradient with 10 mM ammonium formate) to provide the title compound as a beige solid (58 mg, 65% yield). LCMS (ESI): m/z [M+H]⁺ calcd for C₂₅H₂₇F₂N₅O₃: 484.21, found 484.7. ¹H NMR (400 MHz, Methanol-d₄) δ 8.34 (d, J=2.4 Hz, 1H), 7.92 (d, J=2.4 Hz, 1H), 6.86 (ddd, J=11.5, 8.3, 3.1 Hz, 1H), 6.77-6.70 (m, 1H), 5.56-5.40 (m, 2H), 4.43-4.32 (m, 1H), 4.15 (dd, J=12.4, 1.7 Hz, 1H), 4.03-3.92 (m, 2H), 3.68 (s, 1H), 3.62-3.49 (m, 1H), 3.36 (dd, J=9.0, 3.6 Hz, 1H), 2.80-2.53 (m, 2H), 2.20 (d, J=14.0 Hz, 1H), 2.14-1.68 (m, 7H), 1.55-1.26 (m, 4H).

### Synthesis of Compound 31: 3-((((2¹S,2⁴S,5²R,5³S)-13,15-difluoro-6-oxo-3,8-dioxa-5(2,1)-piperidina-1(1,2)-benzena-2(1,4)-cyclohexanacyclooctaphane-5³-yl)amino)methyl)oxetane-3-carbonitrile

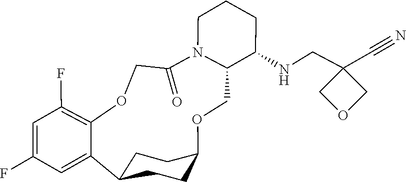 [SMILES: N#CC1(CN[C@H]2CCCN3C(=O)COc4c(F)cc(F)cc4[C@H]4CC[C@H](CC4)OC[C@@H]23)COC1]

### Step 1: Preparation of (3-cyanooxetan-3-yl)methyl methanesulfonate

To a solution of 3-(hydroxymethyl)oxetane-3-carbonitrile (40.0 mg, 354 μmol) in dichloromethane (1.2 mL) was added triethylamine (73.4 μL, 530 μmol) followed by slow addition of methanesulfonyl chloride (28.7 μL, 371 μmol). The reaction was stirred at room temperature for 1 h then was diluted with dichloromethane and brine (10 mL/5 mL), then phases were separated, and organic phase was washed with brine again. The organic layer was dried over sodium sulfate, filtered, and concentrated at low temperature to near dryness to provide the title compound, which was used without further purification. ¹H NMR (400 MHz, CDCl₃) δ 4.98 (d, J=7.0 Hz, 2H), 4.64 (s, 2H), 4.58 (d, J=7.0 Hz, 2H), 3.14 (d, J=2.7 Hz, 3H).

### Step 2: Preparation of 3-((((2¹S,2⁴S,5²R,5³S)-13,15-difluoro-6-oxo-3,8-dioxa-5(2,1)-piperidina-1(1,2)-benzena-2(1,4)-cyclohexanacyclooctaphane-5³-yl)amino)methyl)oxetane-3-carbonitrile (Compound 31)

To a solution of Intermediate 4 (80.0 mg, 210 μmol) in ethanol (0.6 mL) was added (3-cyanooxetan-3-yl)methyl methanesulfonate (60.3 mg, 315 μmol) followed by N,N-diisopropylethylamine (77.7 μL, 442 μmol). The reaction was stirred at 120° C. for 2 days. The reaction mixture was concentrated and the resulting residue was purified by preparative HPLC (C18 column, 0-80% acetonitrile/water gradient with 10 mM ammonium formate) to provide the title compound (50 mg, 50% yield). LCMS (ESI): m/z [M+H]⁺ calcd for C₂₅H₃₁F₂N₃O₄: 476.23. found 476.3. ¹H NMR (400 MHz, Methanol-d₄) δ 6.85 (ddd, J=11.5, 8.3, 3.1 Hz, 1H), 6.78-6.69 (m, 1H), 5.46 (dd, J=12.4, 3.2 Hz, 1H), 5.26-5.17 (m, 1H), 4.87-4.83 (m, 2H), 4.59 (dd, J=7.9, 6.5 Hz, 2H), 4.14 (dd, J=12.4, 1.7 Hz, 1H), 3.97-3.81 (m, 2H), 3.70 (s, 1H), 3.55-3.42 (m, 2H), 3.29 (d, J=2.1 Hz, 2H), 2.96-2.85 (m, 1H), 2.73-2.56 (m, 2H), 2.21-2.02 (m, 2H), 1.94-1.79 (m, 3H), 1.70-1.28 (m, 7H).

### Preparation of Compound 32: 2-(((2¹S,2⁴S,5²R,5³S)-13,15-difluoro-6-oxo-3,8-dioxa-5(2,1)-piperidina-1(1,2)-benzena-2(1,4)-cyclohexanacyclooctaphane-5³-yl)amino)-6-(trifluoromethyl)nicotinonitrile

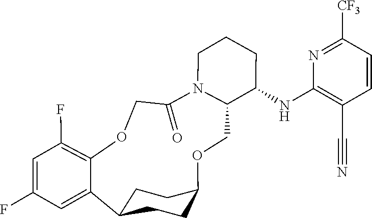 [SMILES: N#Cc1ccc(C(F)(F)F)nc1N[C@H]1CCCN2C(=O)COc3c(F)cc(F)cc3[C@H]3CC[C@H](CC3)OC[C@@H]12]

A solution of Intermediate 4 (50.0 mg, 131 μmol), N,N-diisopropylethylamine (46.2 μL, 263 μmol) and 2-chloro-6-(trifluoromethyl)nicotinonitrile (55.4 mg, 263 μmol) in N-methyl-2-pyrrolidone (340 L) was stirred at 130° C. for 5 h. The crude reaction mixture was directly loaded and purified by preparative HPLC (C18 column, 40-100% acetonitrile/water with 10 mM ammonium formate) to provide the title compound as a beige solid (55 mg, 76% yield). LCMS (ESI): m/z [M+H]⁺ calcd for C₂₇H₂₇F₅N₄O₃: 551.20, found 551.7. ¹H NMR (400 MHz, DMSO-d₆) δ 8.25 (d, J=7.8 Hz, 1H), 7.55 (d, J=7.4 Hz, 1H), 7.22-7.10 (m, 2H), 6.88 (d, J=9.2 Hz, 1H), 5.44 (dd, J=12.2, 3.0 Hz, 1H), 5.31-5.19 (m, 1H), 4.27-4.13 (m, 1H), 3.98 (d, J=12.0 Hz, 1H), 3.89-3.73 (m, 2H), 3.62 (s, 1H), 3.36-3.22 (m, 1H), 2.64-2.56 (m, 2H), 2.17-1.88 (m, 3H), 1.88-1.68 (m, 2H), 1.68-1.50 (m, 2H), 1.46-1.16 (m, 4H).

### Synthesis of Compound 33: 2-((((21S,24S,52R,53S)-6-oxo-3,8-dioxa-5(2,1)-piperidina-1(1,2)-benzena-2(1,4)-cyclohexanacyclooctaphane-53-yl)amino)methyl)oxetane-2-carbonitrile

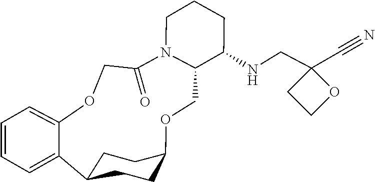 [SMILES: N#CC1(CN[C@H]2CCCN3C(=O)COc4ccccc4[C@H]4CC[C@H](CC4)OC[C@@H]23)CCO1]

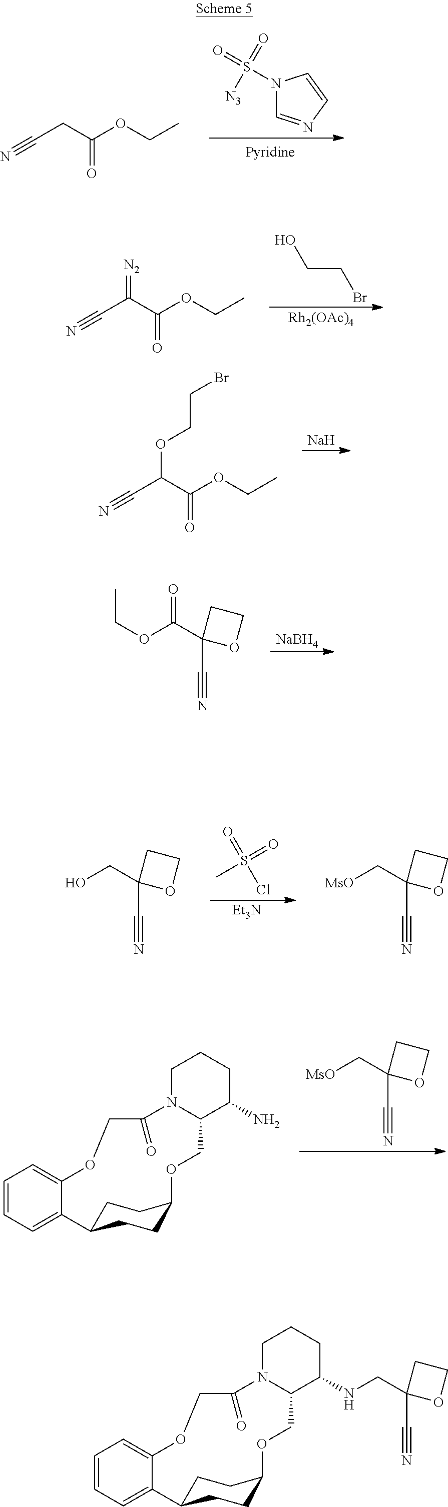

### Step 1: Preparation of ethyl 2-cyano-2-diazoacetate

To ethyl cyanoacetate (0.47 mL, 4.33 mmol) in acetonitrile (21.7 mL) were added 1H-imidazole-1-sulfonyl azide (1.09 g, 5.20 mmol) and pyridine (1.76 mL, 21.7 mmol). The reaction mixture was stirred at 40° C. for 18 h. The mixture was then diluted with ethyl acetate, washed with 1 M hydrochloric acid and brine, dried over sodium sulfate, filtered, and concentrated. The crude product was purified by silica gel chromatography (0-100% ethyl acetate/heptanes gradient) to provide the title compound as an oil (255 mg, 37% yield). ¹H NMR (400 MHz, CDCl₃) δ 4.34 (q, J=7.1 Hz, 2H), 1.34 (t, J=7.1 Hz, 3H).

### Step 2: Preparation of ethyl 2-(2-bromoethoxy)-2-cyanoacetate

To a solution of ethyl 2-cyano-2-diazoacetate (225 mg, 1.62 mmol) in dichloromethane (14.7 mL), 2-bromoethanol (110 μL, 1.47 mmol) was added followed by rhodium (II) acetate dimer (33.5 mg, 73.5 μmol). The reaction mixture was stirred at room temperature for 21 h. Water was added and the mixture was extracted dichloromethane (20 mL×2). The combined organic phases were dried over sodium sulfate, filtered, and concentrated to provide the title compound as a yellow oil (340 mg, 98% yield). This material was used without further purification. ¹H NMR (400 MHz, CDCl₃) δ 4.94 (s, 1H), 4.36 (q, J=7.1 Hz, 2H), 4.06 (ddt, J=35.6, 10.3, 6.1 Hz, 2H), 3.54 (t, J=6.1 Hz, 2H), 1.36 (t, J=7.2 Hz, 3H).

### Step 3: Preparation of ethyl 2-cyanooxetane-2-carboxylate

To ethyl 2-(2-bromoethoxy)-2-cyanoacetate (300 mg, 1.27 mmol) in N,N-dimethylformamide (40.5 mL) at 0° C. was added sodium hydride (60% in mineral oil, 61.0 mg, 1.53 mmol) and the mixture was stirred for 1 h at 0° C. The reaction was poured into 10% aqueous ammonium chloride and extracted with dichloromethane (3×7 mL). The combined organic phases were dried over magnesium sulfate, filtered, and concentrated onto silica gel. This material was dry-loaded and purified by silica gel chromatography (0-50% ethyl acetate/heptanes gradient) to provide the title compound as a colorless oil (120 mg, 61% yield). ¹H NMR (400 MHz, CDCl₃) δ 4.87 (ddd, J=8.6, 7.1, 5.8 Hz, 1H), 4.77 (ddd, J=8.6, 6.6, 5.9 Hz, 1H), 4.37 (q, J=7.2 Hz, 2H), 3.23 (dddd, J=32.2, 12.0, 8.6, 6.9 Hz, 1H), 1.37 (t, J=7.1 Hz, 3H).

### Step 4: Preparation of 2-(hydroxymethyl)oxetane-2-carbonitrile

To ethyl 2-cyanooxetane-2-carboxylate (120 mg, 773 μmol) in methanol (7.7 mL) was added sodium borohydride (152 mg, 3.87 mmol) and the reaction was stirred for at room temperature for 18 h. Water was added and the reaction was extracted with dichloromethane (30 mL), dried over sodium sulfate, filtered, and concentrated to provide the title compound, which was used without further purification (60.0 mg, 69% yield). ¹H NMR (400 MHz, CDCl₃) δ 4.83 (ddd, J=8.7, 7.6, 5.9 Hz, 1H), 4.61-4.48 (m, 1H), 3.90 (d, J=12.7 Hz, 1H), 3.77 (d, J=12.7 Hz, 1H), 3.14 (ddd, J=11.7, 9.0, 7.6 Hz, 1H), 2.96 (ddd, J=11.7, 8.7, 6.0 Hz, 1H).

### Step 5: Preparation of (3-cyanooxetan-3-yl)methyl methanesulfonate

To a solution of 2-(hydroxymethyl)oxetane-2-carbonitrile (55.0 mg, 486 μmol) in dichloromethane (2.43 mL) were added triethylamine (204 μL, 1.46 mmol) and methanesulfonyl chloride (45.3 μL, 583 μmol). The reaction mixture was stirred at room temperature for 1 h, then water was added. The resulting mixture was extracted with dichloromethane (2×10 mL). The combined organic phases were then dried over sodium sulfate, filtered, and concentrated to provide the title compound, which was used without further purification. ¹H NMR (400 MHz, CDCl₃) δ 4.74 (ddd, J=8.6, 7.4, 6.0 Hz, 1H), 4.55 (dt, J=8.8, 6.3 Hz, 1H), 4.44 (d, J=11.7 Hz, 1H), 4.39 (d, J=11.7 Hz, 1H).

### Step 6: Preparation of 2-((((21S,24S,52R,53S)-6-oxo-3,8-dioxa-5(2,1)-piperidina-1(1,2)-benzena-2(1,4)-cyclohexanacyclooctaphane-53-yl)amino)methyl)oxetane-2-carbonitrile (Compound 33)

To solution of Intermediate 2 (60.0 mg. 174 mol) in ethanol (0.5 mL) were added N,N-diisopropylethylamine (61.3 μL, 348 μmol) and (3-cyanooxetan-3-yl)methyl methanesulfonate (46.6 mg, 244 mol). The reaction mixture was stirred at 120° C. for 18 h. The reaction mixture was then cooled and added to water, the aqueous phase was extracted with ethyl acetate (3×), washed with brine, dried over sodium sulfate, filtered, and concentrated. The crude product was purified by preparation HPLC (C18 column, 0-100% acetonitrile/water with 10 mM ammonium formate) to provide the title compound (2.8 mg, 3.7% yield). LCMS (ESI): m/z [M+H]⁺ calcd for C₂₅H₃₃N₃O₄: 440.25, found 440.8. ¹H NMR (400 MHz, Methanol-d₄) δ 7.14 (td, J=7.8, 1.7 Hz, 1H), 7.06 (dd, J=7.4, 1.7 Hz, 1H), 6.90-6.79 (m, 2H), 5.29 (d, J=10.6 Hz, 1H), 5.19 (s, 1H), 4.76 (dt, J=14.2, 7.1 Hz, 1H), 4.62-4.49 (m, 1H), 4.10 (d, J=10.6 Hz, 1H), 3.97-3.88 (m, 1H), 3.79 (d, J=12.8 Hz, 1H), 3.68 (s, 1H), 3.59-3.44 (m, 2H), 3.25-3.09 (m, 2H), 3.07-2.90 (m, 3H), 2.78-2.63 (m, 1H), 2.61-2.50 (m, 1H), 2.34-2.25 (m, 1H), 2.17 (d, J=12.6 Hz, 1H), 1.85 (dd, J=28.3, 14.7 Hz, 3H), 1.65-1.32 (m, 7H).

### Human OX2R IP1 Assay

The G protein-coupled protein receptor (GPCR) OX2R signals through the Gq/11 signaling pathway. Activation of this pathway can be readily detected by measuring the accumulation of inositol monophosphate (IP-one or IP1) in the presence of excess LiCl. This can be quantified using the commercially available cell-based IP-One Gq kit (CisBio cat #: 62IPAPEB) coupled with a cell line expressing OX2R. IP1 generated within the cell by OX2R agonism competes with the IP1 analog coupled to a d2 fluorophore (FRET acceptor) for binding to an anti-IP1 monoclonal antibody labeled with Eu cryptated (FRET donor). The measured HTRF-FRET based signal is inversely proportional to the IP1 concentration produced.

Tetracycline-inducible suspension human embryonic kidney cells (Expi293F Inducible; ThermoFisher #A39241) stably expressing tet-responsive human OX2R were induced with 4 μg/mL doxycycline at 32° C. After 24 hours of induction, cells were harvested, aliquoted, and cryopreserved in Expi293 medium containing 10% DMSO. On the day of the assay, cells are thawed, washed with PBS, and resuspended in 1× Cisbio stimulation buffer. Cells were then plated at a concentration of 6,000 cells/well into a 384-well assay plate containing test compounds to give a final concentration of 0.5% DMSO. Following a one-hour incubation at 37° C., the reaction was terminated by addition of the Cisbio detection mix containing IP1-d2 and anti-IP1-cryptate in lysis buffer. Following a one-hour incubation at room temperature, HTRF-FRET values were measured using a ClarioStar Plus (BMG Labtech) plate reader and the data were expressed as the standard 665 nm/620 nm ratio.

Exemplified compounds, characterization data and assay data are provided in Table 2.

TABLE 2

| | | | | LCMS | pEC50 |
|---|---|---|---|---|---|
| # | Structure | IUPAC | NMR | (MH⁺) | Bin |
| 1 | 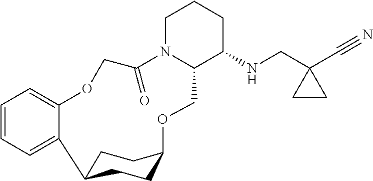 [SMILES: N#CC1(CN[C@H]2CCCN3C(=O)COc4ccccc4[C@H]4CC[C@H](CC4)OC[C@@H]23)CC1] | 1- ((((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)methyl) cyclopropane-1- carbonitrile | ¹H NMR (400 MHz, CD₃OD) δ 7.14 (td, J = 7.7, 1.7 Hz, 1H), 7.07 (dd, J = 7.4, 1.6 Hz, 1H), 6.90-6.80 (m, 2H), 5.29 (d, J = 10.6 Hz, 1H), 5.24-5.14 (m, 1H), 4.11 (d, J = 10.6 Hz, 1H), 3.98-3.88 (m, 1H), 3.85-3.74 (m, 1H), 3.69 (s. 1H), 3.56- 3.40 (m, 2H), 2.99- 2.87 (m, 2H), 2.81- 2.66 (m, 2H), 2.63- 2.50 (m, 1H), 2.41- 2.23 (m, 1H), 2.17 (d, J = 13.2 Hz, 1H). 1.94- 1.73 (m, 3H). 1.70- 1.17 (m, 9H), 1.08- 0.89 (m, 2H). | 424.3 | *** |
| 2 | 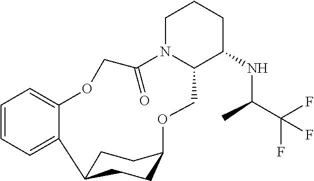 [SMILES: C[C@@H](N[C@H]1CCCN2C(=O)COc3ccccc3[C@H]3CC[C@H](CC3)OC[C@@H]12)C(F)(F)F] | (2¹S,2⁴S,5²R,5³S)-5³- (((R)-1,1,1- trifluoropropan-2- yl)amino)-3,8-dioxa- 5(2,1)-piperidina- 1(1,2)-benzena- 2(1,4)- cyclohexanacyclo- octaphan-6-one | ¹H NMR (400 MHz, DMSO-d₆) δ 7.13 (dd, J = 7.4, 5.9 Hz, 1H), 7.08- 7.03 (m, 1H), 6.81 (t, J = 7.7 Hz, 2H), 5.26 (d, J = 10.4 Hz, 1H), 5.10- 4.93 (m, 1H), 3.92 (d, J = 10.4 Hz, 1H), 3.83- 3.71 (m, 1H), 3.62 (d, J = 18.2 Hz, 2H), 2.86 (d, J = 10.6 Hz, 1H), 2.68- 2.55 (m, 1H), 2.28 (ddd, J = 22.1, 10.5, 6.6 Hz, 3H), 2.08 (d, J = 14.3 Hz, 1H), 1.71-1.59 (m, 3H), 1.51-1.01 (m, 12H) | 441.4 | ** |
| 3 | 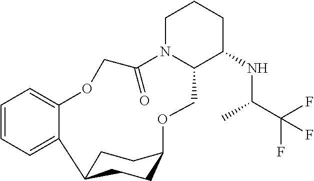 [SMILES: C[C@H](N[C@H]1CCCN2C(=O)COc3ccccc3[C@H]3CC[C@H](CC3)OC[C@@H]12)C(F)(F)F] | (2¹S,2⁴S,5²R,5³S)-5³- (((S)-1,1,1- trifluoropropan-2- yl)amino)-3,8-dioxa- 5(2,1)-piperidina- 1(1,2)-benzena- 2(1,4)- cyclohexanacyclo- octaphan-6-one | ¹H NMR (400 MHz, DMSO-d6) δ 7.13 (d, J = 7.7 Hz, 1H), 7.05 (d, J = 5.8 Hz, 1H), 6.80 (d, J = 8.5 Hz, 2H), 5.26 (d, J = 10.4 Hz, 1H), 4.84- 4.89 (m, 1H), 3.92 (d, J = 10.4 Hz, 1H), 3.79- 3.72 (m, 1H), 3.66- 3.53 (m, 2H), 3.44- 3.37 (m, 1H), 2.76- 2.79 (m, 1H), 2.22- 1.95 (m, 3H), 1.76- 1.61 (m, 3H), 1.08- 1.48 (m, 13H). | 441.4 | ** |
| 4 | 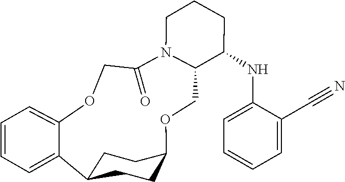 [SMILES: N#Cc1ccccc1N[C@H]1CCCN2C(=O)COc3ccccc3[C@H]3CC[C@H](CC3)OC[C@@H]12] | 2- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)benzonitrile | ¹H NMR (400 MHz, DMSO-d₆) δ 7.50 (t, J = 8.5 Hz, 2H), 7.13 (t, J = 7.7 Hz, 1H), 7.06 (d, J = 7.6 Hz, 1H), 6.89 (d, J = 8.4 Hz, 1H), 6.84-6.80 (m, 2H), 6.72 (t, J = 7.5 Hz, 1H), 5.70 (d, J = 7.3 Hz, 1H), 5.30 (d, J = 10.4 Hz, 1H), 5.20- 5.11 (m, 1H), 3.93 (dd, J = 24.1, 10.5 Hz, 2H), 3.71 (d, J = 10.5 Hz, 2H), 3.58 (s, 1H), 3.19 (dd, J = 23.0, 16.9 Hz, 2H), 2.68-2.57 (m, 1H), 2.25-2.06 (m, 2H), 1.91 (dd, J = 24.0, 12.7 Hz, 1H), 1.79 (d, J = 10.9 Hz, 2H), 1.63- 1.52 (m, 2H), 1.39- 1.06 (m, 5H). | 446.4 | *** |
| 5 | 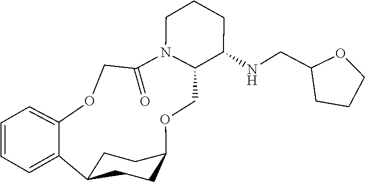 [SMILES: O=C1COc2ccccc2[C@H]2CC[C@H](CC2)OC[C@H]2[C@@H](NCC3CCCO3)CCCN12] | (2¹S,2⁴S,5²R,5³S)-5³- (((tetrahydrofuran-2- yl)methyl)amino)- 3,8-dioxa-5(2,1)- piperidina-1(1,2)- benzena-2(1,4)- cyclohexanacyclo- octaphan-6-one | ¹H NMR (400 MHz, CD₃OD) δ 7.18-7.12 (m, 1H), 7.08 (dd, J = 7.4, 1.7 Hz, 1H), 6.86 (td, J = 7.4, 1.1 Hz, 1H), 6.80 (dd, J = 8.1, 1.0 Hz, 1H), 5.17 (d, J = 10.3 Hz, 1H), 5.08 (dq, J = 9.1, 4.5 Hz, 1H), 4.03 (d, J = 10.3 Hz, 1H), 3.96-3.72 (m, 3H), 3.71-3.61 (m, 3H), 3.46-3.28 (m, 2H), 2.83-2.67 (m, 4H), 2.67-2.50 (m, 2H), 2.36-2.21 (m, 1H), 1.89-1.80 (m, 2H), 1.80-1.65 (m, 4H), 1.65-1.16 (m, 8H) | 429.5 | ** |
| 6 | 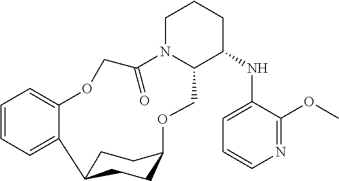 [SMILES: COc1ncccc1N[C@H]1CCCN2C(=O)COc3ccccc3[C@H]3CC[C@H](CC3)OC[C@@H]12] | (2¹S,2⁴S,5²R,5³S)-5³- ((2-methoxypyridin- 3-yl)amino)-3,8- dioxa-5(2,1)- piperidina-1(1,2)- benzena-2(1,4)- cyclohexanacyclo- octaphan-6-one | ¹H NMR (400 MHz, CD₃OD) δ 7.40 (dd, J = 5.1, 1.4 Hz, 1H), 7.14 (td, J = 7.8, 1.6 Hz, 1H), 7.06 (d, J = 7.5 Hz, 2H), 6.85 (td, J = 7.1, 1.5 Hz, 3H), 5.42-5.35 (m, 1H), 5.32 (d, J = 10.6 Hz, 1H), 4.12 (d, J = 10.6 Hz, 1H), 4.03- 3.98 (m, 1H), 3.96 (s, 3H), 3.84 (d, J = 13.9 Hz, 1H), 3.73-3.66 (m, 1H), 3.62 (s, 1H), 3.55 (td, J = 13.4, 2.5 Hz, 1H), 2.73 (qd, J = 13.1, 4.5 Hz, 1H), 2.60-2.49 (m, 1H), 2.26 (ddd, J = 26.1, 13.1, 4.2 Hz, 1H), 2.17 (d, J = 14.0 Hz, 1H), 1.97-1.90 (m, 2H), 1.89-1.70 (m, 3H), 1.47-1.29 (m, 3H), 1.29-1.19 (m, 1H). | 452.4 | ** |
| 7 | 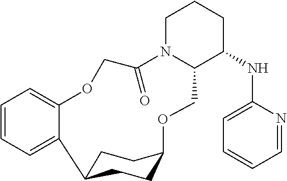 [SMILES: O=C1COc2ccccc2[C@H]2CC[C@H](CC2)OC[C@H]2[C@@H](Nc3ccccn3)CCCN12] | (2¹S,2⁴S,5²R,5³S)-5³- (pyridin-2-ylamino)- 3,8-dioxa-5(2,1)- piperidina-1(1,2)- benzena-2(1,4)- cyclohexanacyclo- octaphan-6-one | ¹H NMR (400 MHz, CD₃OD) δ 8.01-7.96 (m, 1H), 7.45 (ddd, J = 8.7, 7.1, 1.9 Hz, 1H), 7.14 (td, J = 7.8, 1.7 Hz, 1H), 7.06 (dd, J = 7.5, 1.4 Hz, 1H), 6.86 (dd, J = 11.4, 4.4 Hz, 2H), 6.61-6.55 (m, 2H), 5.47-5.39 (m, 1H), 5.32 (d, J = 10.5 Hz, 1H), 4.10 (dd, J = 10.0, 7.0 Hz, 2H), 3.98 (dd, J = 10.9, 9.1 Hz, 1H), 3.84 (d, J = 13.2 Hz, 1H), 3.63 (d, J = 3.7 Hz, 1H), 3.55 (t, J = 12.9 Hz, 1H), 3.38 (dd, J = 9.0, 3.9 Hz, 1H), 2.74 (qd, J = 13.2, 4.4 Hz, 1H), 2.55 (ddd, J = 17.3, 12.0, 5.2 Hz, 1H), 2.27 (qd, J = 13.0, 4.1 Hz, 1H), 2.16 (d, J = 13.9 Hz, 1H), 1.96- 1.86 (m, 2H), 1.80- 1.71 (m, 3H), 1.45- 1.37 (m, 2H), 1.33 (dd, J = 13.7, 4.3 Hz, 1H), 1.27-1.19 (m, 1H). | 422.4 | ** |
| 8 | 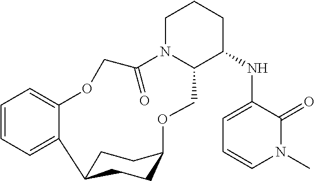 [SMILES: Cn1cccc(N[C@H]2CCCN3C(=O)COc4ccccc4[C@H]4CC[C@H](CC4)OC[C@@H]23)c1=O] | (2¹S,2⁴S,5²R,5³S)-5³- ((1-methyl-2-oxo- 1,2-dihydropyridin- 3-yl)amino)-3,8- dioxa-5(2,1)- piperidina-1(1,2)- benzena-2(1,4)- cyclohexanacyclo- octaphan-6-one | ¹H NMR (400 MHz, DMSO-d6) δ 7.13 (t, J = 7.7 Hz, 1H), 7.05 (d, J = 7.4 Hz, 1H), 6.94 (d, J = 6.7 Hz, 1H), 6.82 (dd, J = 7.1, 5.6 Hz, 2H), 6.34 (d, J = 7.3 Hz, 1H), 6.17 (t, J = 7.0 Hz, 1H), 5.29 (d, J = 10.4 Hz, 1H), 5.16 (dd, J = 15.7, 7.0 Hz, 2H), 3.95 (d, J = 10.4 Hz, 1H), 3.91-3.84 (m, 1H), 3.69 (d, J = 13.1 Hz, 1H), 3.52 (d, J = 10.3 Hz, 2H), 3.45 (s, 3H), 3.04 (dd, J = 9.1, 3.4 Hz, 1H), 2.68-2.52 (m, 2H), 2.24-2.05 (m, 2H), 1.90-1.73 (m, 3H), 1.57 (d, J = 12.5 Hz, 2H), 1.32 (t, J = 10.7 Hz, 2H), 1.22 (dd, J = 15.6, 11.8 Hz, 1H), 1.11 (d, J = 11.1 Hz, 1H). | 452.4 | * |
| 9 | 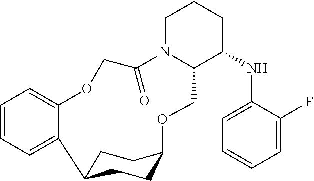 [SMILES: O=C1COc2ccccc2[C@H]2CC[C@H](CC2)OC[C@H]2[C@@H](Nc3ccccc3F)CCCN12] | (2¹S,2⁴S,5²R,5³S)-5³- ((2- fluorophenyl)amino)- 3,8-dioxa-5(2,1)- piperidina-1(1,2)- benzena-2(1,4)- cyclohexanacyclo- octaphan-6-one | ¹H NMR (400 MHz, CD₃OD) δ 7.13 (t, J = 7.8 Hz, 1H), 7.08-6.92 (m, 4H), 6.88-6.81 (m, 2H), 6.62 (dt, J = 13.6, 4.0 Hz, 1H), 5.45-5.36 (m, 1H), 5.30 (d, J = 10.6 Hz, 1H), 4.12 (d, J = 10.6 Hz, 1H), 4.02- 3.94 (m, 1H), 3.81 (d, J = 12.2 Hz, 1H), 3.77- 3.67 (m, 1H), 3.61 (s, 1H), 3.53 (dd, J = 18.8, 7.6 Hz, 1H), 3.33 (dd, J = 9.1, 3.9 Hz, 1H), 2.73 (qd, J = 13.1, 4.4 Hz, 1H), 2.59-2.47 (m, 1H), 2.26 (qd, J = 13.0, 4.1 Hz, 1H), 2.16 (d, J = 13.7 Hz, 1H), 1.90 (dd, J = 21.9, 9.5 Hz, 2H), 1.81 (dd, J = 12.4, 3.5 Hz, 1H), 1.77-1.68 (m, 2H), 1.41 (dd, J = 13.2, 3.4 Hz, 2H), 1.36- 1.28 (m, 1H), 1.27- 1.18 (m, 1H). | 439.3 | *** |
| 10 | 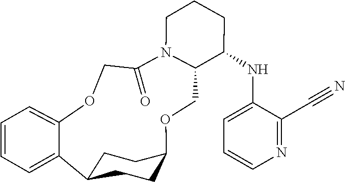 [SMILES: N#Cc1ncccc1N[C@H]1CCCN2C(=O)COc3ccccc3[C@H]3CC[C@H](CC3)OC[C@@H]12] | 3- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino) picolinonitrile | ¹H NMR (400 MHz, CD₃OD) δ 7.97-7.91 (m, 1H), 7.54 (d, J = 8.8 Hz, 1H), 7.51-7.45 (m, 1H), 7.14 (t, J = 7.7 Hz, 1H), 7.06 (d, J = 7.3 Hz, 1H), 6.86 (t, J = 8.5 Hz, 2H), 5.32 (dt, J = 10.2, 5.1 Hz, 2H), 4.13 (dd, J = 10.6, 2.0 Hz, 1H), 4.05 (t, J = 9.5 Hz, 1H), 3.86 (d, J = 12.6 Hz, 2H), 3.66 (s, 1H), 3.56 (t, J = 13.3 Hz, 1H), 3.37-3.32 (m, 1H), 2.71 (dt, J = 22.3, 11.0 Hz, 1H), 2.56 (dd, J = 14.5, 9.0 Hz, 1H), 2.32- 2.13 (m, 2H), 1.96 (dd, J = 13.6, 7.6 Hz, 4H), 1.75 (d, J = 12.8 Hz, 2H), 1.44 (d, J = 13.2 Hz, 2H), 1.40- 1.30 (m, 2H), 1.24 (d, J = 12.5 Hz, 1H). | 447.3 | *** |
| 11 | 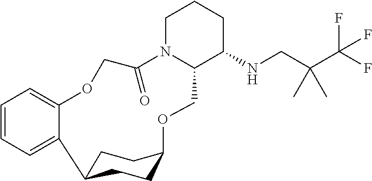 [SMILES: CC(C)(CN[C@H]1CCCN2C(=O)COc3ccccc3[C@H]3CC[C@H](CC3)OC[C@@H]12)C(F)(F)F] | (2¹S,2⁴S,5²R,5³S)-5³- ((3,3,3-trifluoro-2,2- dimethylpropyl)amino)- 3,8-dioxa-5(2,1)- piperidina-1(1,2)- benzena-2(1,4)- cyclohexanacyclo- octaphan-6-one | ¹H NMR (400 MHz, CD₃OD) δ 7.13 (td, J = 7.8, 1.4 Hz, 1H), 7.06 (dd, J = 7.4, 1.4 Hz, 1H), 6.88-6.79 (m, 2H), 5.28 (d, J = 10.6 Hz, 1H), 5.21-5.13 (m, 1H), 4.10 (d, J = 10.6 Hz, 1H), 3.95-3.87 (m, 1H), 3.77 (d, J = 14.4 Hz, 1H), 3.67 (s, 1H), 3.54-3.41 (m, 2H), 2.80-2.68 (m, 4H), 2.55 (ddd, J = 17.5, 12.0, 5.2 Hz, 1H), 2.30 (qd, J = 13.1, 4.1 Hz, 1H), 2.16 (d, J = 14.1 Hz, 1H), 1.87-1.78 (m, 3H), 1.67-1.57 (m, 1H), 1.43 (qdd, J = 18.3, 15.7, 5.2 Hz, 5H), 1.30- 1.23 (m, 1H), 1.15 (s, 6H). | 469.3 | *** |
| 12 | 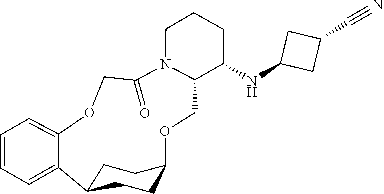 [SMILES: N#C[C@H]1C[C@H](N[C@H]2CCCN3C(=O)COc4ccccc4[C@H]4CC[C@H](CC4)OC[C@@H]23)C1] | (1S,3r)-3- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)cyclobutane- 1-carbonitrile | ¹H NMR (400 MHz, CD3OD) δ 7.19-7.11 (m, 1H), 7.07 (d, J = 7.4 Hz, 1H), 6.85 (dd, J = 13.6, 7.4 Hz, 2H), 5.29 (d, J = 10.6 Hz, 1H), 5.15-5.05 (m, 1H), 4.10 (d, J = 10.6 Hz, 1H), 3.96-3.87 (m, 1H), 3.87-3.73 (m, 2H), 3.69 (s, 1H), 3.55- 3.39 (m, 2H), 3.17- 3.10 (m, 1H), 2.89- 2.64 (m, 2H), 2.64- 2.49 (m, 3H), 2.40- 2.21 (m, 2H), 2.16 (d, J = 14.7 Hz, 1H), 1.89- 1.75 (m, 3H), 1.68- 1.37 (m, 5H), 1.36- 1.23 (m, 2H), 0.89 (d, J = 7.6 Hz, 1H) | 424.3 | * |
| 13 | 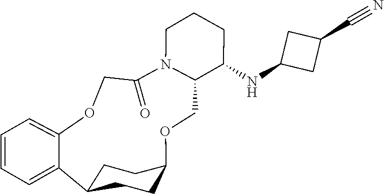 [SMILES: N#C[C@H]1C[C@@H](N[C@H]2CCCN3C(=O)COc4ccccc4[C@H]4CC[C@H](CC4)OC[C@@H]23)C1] | (1R,3s)-3- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)cyclobutane- 1-carbonitrile | ¹H NMR (400 MHz, CD₃OD) δ 7.18-7.10 (m, 1H), 7.07 (dd, J = 7.4, 1.7 Hz, 1H), 6.89- 6.79 (m, 2H), 5.29 (d, J = 10.6 Hz, 1H), 5.13- 5.03 (m, 1H), 4.10 (d, J = 10.6 Hz, 1H), 3.95- 3.85 (m, 1H), 3.78 (d, J = 10.8 Hz, 1H), 3.68 (s, 1H), 3.54-3.38 (m, 3H), 2.99-2.79 (m, 2H), 2.77-2.63 (m, 3H), 2.62-2.50 (m, 1H), 2.36-2.22 (m, 1H), 2.15 (ddd, J = 19.2, 13.9, 8.3 Hz, 3H), 1.89- 1.72 (m, 3H), 1.68- 1.21 (m, 7H). | 424.3 | ** |
| 14 | 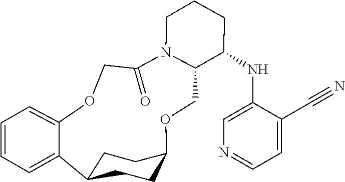 [SMILES: N#Cc1ccncc1N[C@H]1CCCN2C(=O)COc3ccccc3[C@H]3CC[C@H](CC3)OC[C@@H]12] | 3- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino) isonicotinonitrile | ¹H NMR (400 MHz, CD₃OD) δ 8.45 (s, 1H), 7.94 (d, J = 4.7 Hz, 1H), 7.44 (d, J = 5.0 Hz, 1H), 7.15 (td, J = 7.7, 1.7 Hz, 1H), 7.07 (dd, J = 7.3, 1.6 Hz, 1H), 6.87 (dd, J = 12.1, 4.6 Hz, 2H), 5.44-5.36 (m, 1H), 5.33 (d, J = 10.6 Hz, 1H), 4.14 (d, J = 10.6 Hz, 1H), 4.11-3.98 (m, 2H), 3.91-3.77 (m, 1H), 3.67 (s, 1H), 3.63- 3.51 (m, 1H), 3.36 (dd, J = 9.1, 3.7 Hz, 1H), 2.80-2.65 (m, 1H), 2.62-2.48 (m, 1H), 2.42-2.12 (m, 2H), 2.08-1.89 (m, 3H), 1.87-1.69 (m, 2H), 1.56-1.15 (m, 4H). | 447.3 | *** |
| 15 | 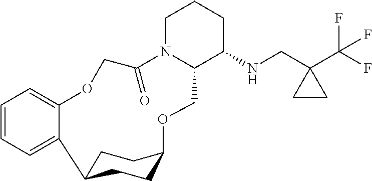 [SMILES: O=C1COc2ccccc2[C@H]2CC[C@H](CC2)OC[C@H]2[C@@H](NCC3(C(F)(F)F)CC3)CCCN12] | (2¹S,2⁴S,5²R,5³S)-5³- (((1- (trifluoromethyl) cyclopropyl) methyl)amino)- 3,8-dioxa-5(2,1)- piperidina-1(1,2)- benzena-2(1,4)- cyclohexanacyclo- octaphan-6-one | ¹H NMR (400 MHz, DMSO-d₆) δ 7.10 (ddd, J = 23.1, 12.4, 7.6 Hz, 2H), 6.81 (t, J = 8.4 Hz, 2H), 5.25 (d, J = 10.4 Hz, 1H), 5.04-4.92 (m, 1H), 3.92 (d, J = 10.4 Hz, 1H), 3.79-3.71 (m, 1H), 3.67-3.56 (m, 2H), 2.70 (ddt, J = 35.8, 32.1, 13.5 Hz, 5H), 2.31- 2.03 (m, 3H), 1.68 (dd, J = 26.3, 14.2 Hz, 3H), 1.50-1.07 (m, 8H), 0.90-0.79 (m, 4H). | 467.3 | *** |
| 16 | 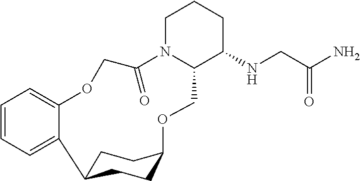 [SMILES: NC(=O)CN[C@H]1CCCN2C(=O)COc3ccccc3[C@H]3CC[C@H](CC3)OC[C@@H]12] | 2- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)acetamide | ¹H NMR (400 MHz, CD₃OD) δ 7.14 (td, J = 7.8, 1.7 Hz, 1H), 7.06 (dd, J = 7.4, 1.6 Hz, 1H), 6.89-6.80 (m, 2H), 5.28 (d, J = 10.6 Hz, 1H), 5.20 (s, 1H), 4.10 (d, J = 10.6 Hz, 1H), 3.95 (t, J = 8.9 Hz, 1H), 3.79 (d, J = 13.4 Hz, 1H), 3.70 (s, 1H), 3.60-3.35 (m, 4H), 2.94 (d, J = 10.0 Hz, 1H), 2.72 (qd, J = 12.9, 4.3 Hz, 1H), 2.61-2.51 (m, 1H), 2.29 (qd, J = 13.0, 4.1 Hz, 1H), 2.16 (d, J = 14.0 Hz, 1H), 2.01-1.75 (m, 3H), 1.69-1.51 (m, 2H), 1.52-1.31 (m, 4H), 1.30-1.22 (m, 1H). | 402.3 | ** |
| 17 | 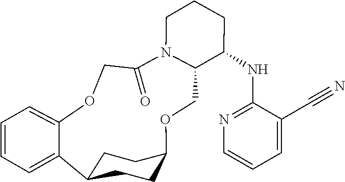 [SMILES: N#Cc1cccnc1N[C@H]1CCCN2C(=O)COc3ccccc3[C@H]3CC[C@H](CC3)OC[C@@H]12] | 2- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)nicotinonitrile | ¹H NMR (400 MHz, DMSO-d₆) δ 8.34 (dd, J = 4.9, 1.9 Hz, 1H), 7.95 (dd, J = 7.6, 1.9 Hz, 1H), 7.15 (td, J = 7.9, 1.7 Hz, 1H), 7.11-7.04 (m, 1H), 6.89-6.79 (m, 3H), 6.73 (dd, J = 7.6, 4.9 Hz, 1H), 5.32 (d, J = 10.3 Hz, 1H), 5.29- 5.20 (m, 1H), 4.34- 4.18 (m, 1H), 3.98- 3.86 (m, 2H), 3.71 (d, J = 11.8 Hz, 1H), 3.60 (s, 1H), 3.22 (dd, J = 9.4, 3.8 Hz, 1H), 2.69-2.58 (m, 2H), 2.27-2.03 (m, 2H), 1.96 (t, J = 11.3 Hz, 1H), 1.88-1.67 (m, 2H), 1.67-1.45 (m, 2H), 1.43-1.04 (m, 4H). | 447.2 | *** |
| 18 | 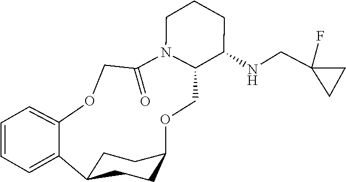 [SMILES: O=C1COc2ccccc2[C@H]2CC[C@H](CC2)OC[C@H]2[C@@H](NCC3(F)CC3)CCCN12] | (2¹S,2⁴S,5²R,5³S)-5³- (((1- fluorocyclopropyl) methyl)amino)-3,8- dioxa-5(2,1)- piperidina-1(1,2)- benzena-2(1,4)- cyclohexanacyclo- octaphan-6-one | ¹H NMR (400 MHz, DMSO-d₆) δ 9.04 (s, 1H), 7.24-7.04 (m, 2H), 6.95-6.78 (m, 2H), 5.32-5.25 (m, 1H), 4.00-3.44 (m, 5H), 2.66-2.61 (m, 1H), 2.38- 0.07 (m, 21H). | 417.4 | ** |
| 19 | 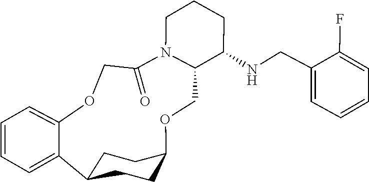 [SMILES: O=C1COc2ccccc2[C@H]2CC[C@H](CC2)OC[C@H]2[C@@H](NCc3ccccc3F)CCCN12] | (2¹S,2⁴S,5²R,5³S)-5³- ((2- fluorobenzyl)amino)- 3,8-dioxa-5(2,1)- piperidina-1(1,2)- benzena-2(1,4)- cyclohexanacyclo- octaphan-6-one | ¹H NMR (400 MHz, DMSO-d₆) δ 7.48-4.43 (m, 1H), 7.21-7.12 (m, 4H), 7.06 (d, J = 7.1 Hz, 1H), 6.81 (t, J = 7.7 Hz, 2H), 5.26 (d, J = 9.5 Hz, 1H), 5.05 (s, 1H), 3.97- 3.58 (m, 6H), 3.45- 3.40 (m, 1H), 2.80- 2.62 (m, 2H), 2.33- 2.01 (m, 3H), 1.78- 1.61 (m, 3H), 1.42-1.16 (m, 5H). | 453.5 | ** |
| 20 | 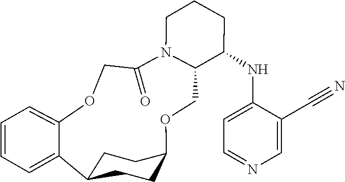 [SMILES: N#Cc1cnccc1N[C@H]1CCCN2C(=O)COc3ccccc3[C@H]3CC[C@H](CC3)OC[C@@H]12] | 4- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)nicotinonitrile | ¹H NMR (400 MHz, CD₃OD) δ 8.41 (s, 1H), 8.28 (d, J = 6.2 Hz, 1H), 7.14 (td, J = 7.7, 1.6 Hz, 1H), 7.06 (dd, J = 7.3, 1.6 Hz, 1H), 7.01 (d, J = 6.4 Hz, 1H), 6.89-6.81 (m, 2H), 5.32 (dd, J = 9.9, 6.4 Hz, 2H), 4.13 (d, J = 10.7 Hz, 1H), 4.08-4.01 (m, 1H), 4.01-3.93 (m, 1H), 3.90-3.82 (m, 1H), 3.66 (s, 1H), 3.55 (td, J = 13.4, 2.3 Hz, 1H), 3.32 (d, J = 4.4 Hz, 1H), 2.72 (qd, J = 13.0, 4.3 Hz, 1H), 2.61-2.50 (m, 1H), 2.25 (ddd, J = 32.2, 20.7, 9.6 Hz, 2H), 2.09- 1.89 (m, 3H), 1.84- 1.69 (m, 2H), 1.48- 1.30 (m, 3H), 1.29- 1.20 (m, 1H). | 447.3 | *** |
| 21 | 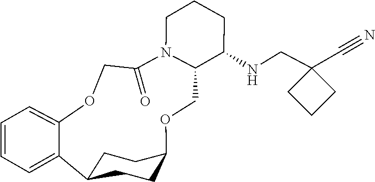 [SMILES: N#CC1(CN[C@H]2CCCN3C(=O)COc4ccccc4[C@H]4CC[C@H](CC4)OC[C@@H]23)CCC1] | 1- ((((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)methyl) cyclobutane-1- carbonitrile | ¹H NMR (400 MHz, CD₃OD) δ 7.14 (td, J = 7.8, 1.7 Hz, 1H), 7.06 (dd, J = 7.4, 1.6 Hz, 1H), 6.87-6.81 (m, 2H), 5.29 (d, J = 10.6 Hz, 1H), 5.25-5.17 (m, 1H), 4.10 (d, J = 10.6 Hz, 1H), 3.92 (dd, J = 11.0, 9.5 Hz, 1H), 3.79 (d, J = 12.9 Hz, 1H), 3.68 (s, 1H), 3.51 (ddd, J = 15.9, 11.2, 3.4 Hz, 2H), 3.02 (d, J = 6.3 Hz, 2H), 2.94-2.87 (m, 1H), 2.74 (ddd, J = 25.6, 12.9, 4.5 Hz, 1H), 2.56 (ddd, J = 17.5, 12.1, 5.3 Hz, 1H), 2.51-2.40 (m, 2H), 2.37-2.27 (m, 1H), 2.14 (ddddd, J = 25.2, 18.3, 9.2, 4.9, 2.5 Hz, 5H), 1.90-1.76 (m, 3H), 1.73-1.22 (m, 7H) | 438.3 | *** |
| 22 | 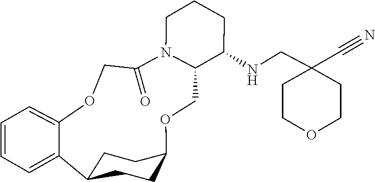 [SMILES: N#CC1(CN[C@H]2CCCN3C(=O)COc4ccccc4[C@H]4CC[C@H](CC4)OC[C@@H]23)CCOCC1] | 4- ((((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)methyl) tetrahydro-2H-pyran-4- carbonitrile | ¹H NMR (400 MHz, CD₃OD) δ 7.14 (td, J = 7.8, 1.7 Hz, 1H), 7.06 (dd, J = 7.4, 1.6 Hz, 1H), 6.88-6.81 (m, 2H), 5.29 (d, J = 10.6 Hz, 1H), 5.23-5.15 (m, 1H), 4.10 (d, J = 10.6 Hz, 1H), 4.00-3.88 (m, 3H), 3.78 (d, J = 14.9 Hz, 1H), 3.71-3.60 (m, 3H), 3.56-3.44 (m, 2H), 2.92-2.83 (m, 3H), 2.73 (qd, J = 12.7, 4.4 Hz, 1H), 2.61-2.51 (m, 1H), 2.31 (ddd, J = 25.9, 13.0, 4.2 Hz, 1H), 2.16 (d, J = 14.2 Hz, 1H), 1.94-1.78 (m, 5H), 1.73-1.22 (m, 9H). | 468.3 | *** |
| 23 | 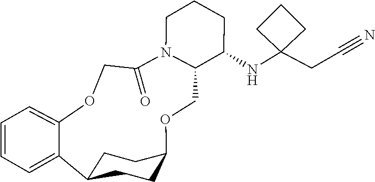 [SMILES: N#CCC1(N[C@H]2CCCN3C(=O)COc4ccccc4[C@H]4CC[C@H](CC4)OC[C@@H]23)CCC1] | 2-(1- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)cyclobutyl) acetonitrile | ¹H NMR (400 MHz, CD₃OD) δ 7.14 (td, J = 7.7, 1.7 Hz, 1H), 7.06 (dd, J = 7.4, 1.6 Hz, 1H), 6.88-6.80 (m, 2H), 5.28 (d, J = 10.6 Hz, 1H), 5.00-4.93 (m, 1H), 4.10 (d, J = 10.6 Hz, 1H), 3.96-3.89 (m, 1H), 3.75 (d, J = 13.6 Hz, 1H), 3.70 (dd, J = 9.0, 3.8 Hz, 2H), 3.52- 3.43 (m, 1H), 2.88 (dt, J = 10.2, 5.0 Hz, 1H), 2.84 (d, J = 1.7 Hz, 2H), 2.78-2.68 (m, 1H), 2.56 (ddd, J = 17.5, 12.1, 5.2 Hz, 1H), 2.31 (ddd, J = 25.8, 13.0, 4.1 Hz, 1H), 2.12 (ddd, J = 18.3, 15.2, 10.4 Hz, 3H), 2.01 (ddd, J = 16.7, 8.2, 4.3 Hz, 2H), 1.89- 1.75 (m, 5H), 1.67- 1.55 (m, 2H), 1.50- 1.39 (m, 2H), 1.34 (dt, J = 13.9, 7.0 Hz, 1H), 1.26 (dd, J = 10.7, 5.5 Hz, 1H). | 438.3 | *** |
| 24 | 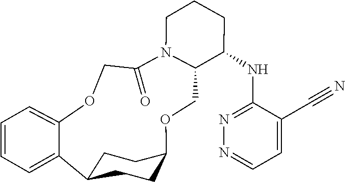 [SMILES: N#Cc1ccnnc1N[C@H]1CCCN2C(=O)COc3ccccc3[C@H]3CC[C@H](CC3)OC[C@@H]12] | 3- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)pyridazine- 4-carbonitrile | ¹H NMR (400 MHz, CD₃OD) δ 8.66 (d, J = 4.9 Hz, 1H), 7.71 (d, J = 4.8 Hz, 1H), 7.14 (td, J = 7.8, 1.7 Hz, 1H), 7.08- 7.04 (m, 1H), 6.88- 6.83 (m, 2H), 5.62- 5.51 (m, 1H), 5.34 (d, J = 10.5 Hz, 1H), 4.67- 4.54 (m, 1H), 4.11 (d, J = 10.5 Hz, 1H), 4.06 (dd, J = 10.8, 9.3 Hz, 1H), 3.87 (d, J = 13.3 Hz, 1H), 3.67 (s, 1H), 3.63-3.52 (m, 1H), 3.39 (dd, J = 9.1, 3.9 Hz, 1H), 2.81-2.69 (m, 1H), 2.61-2.50 (m, 1H), 2.33-2.14 (m, 2H), 2.07-2.00 (m, 1H), 2.00-1.90 (m, 1H), 1.84-1.69 (m, 2H), 1.49-1.19 (m, 5H) | 448.2 | *** |
| 25 | 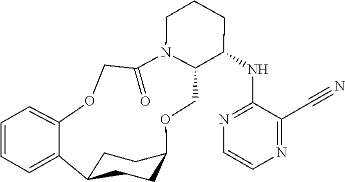 [SMILES: N#Cc1nccnc1N[C@H]1CCCN2C(=O)COc3ccccc3[C@H]3CC[C@H](CC3)OC[C@@H]12] | 3- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)pyrazine-2- carbonitrile | ¹H NMR (400 MHz, CD₃OD) δ 8.34 (d, J = 2.4 Hz, 1H), 7.92 (d, J = 2.4 Hz, 1H), 7.14 (td, J = 7.7, 1.7 Hz, 1H), 7.06 (dd, J = 7.3, 1.6 Hz, 1H), 6.86 (t, J = 7.5 Hz, 2H), 5.52-5.42 (m, 1H), 5.33 (d, J = 10.5 Hz, 1H), 4.38 (dt, J = 12.5, 5.0 Hz, 1H), 4.11 (d, J = 10.6 Hz, 1H), 4.03 (dd, J = 11.0, 9.3 Hz, 1H), 3.85 (d, J = 13.6 Hz, 1H), 3.66 (s, 1H), 3.56 (td, J = 13.4, 2.6 Hz, 1H), 3.35 (dd, J = 9.1, 3.9 Hz, 1H), 2.84- 2.63 (m, 1H), 2.63- 2.41 (m, 1H), 2.33- 2.13 (m, 2H), 2.08- 1.86 (m, 3H), 1.81- 1.64 (m, 2H), 1.50- 1.20 (m, 4H) | 448.2 | *** |
| 26 | 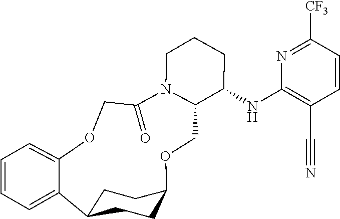 [SMILES: N#Cc1ccc(C(F)(F)F)nc1N[C@H]1CCCN2C(=O)COc3ccccc3[C@H]3CC[C@H](CC3)OC[C@@H]12] | 2- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)- 6- (trifluoromethyl) nicotinonitrile | 1H NMR (400 MHz, DMSO) δ 8.25 (d, J = 7.8 Hz, 1H), 7.50 (d, J = 7.3 Hz, 1H), 7.20-7.12 (m, 2H), 7.11-6.97 (m, 1H), 6.91-6.73 (m, 2H), 5.37-5.18 (m, 2H), 4.21 (d, J = 4.9 Hz, 1H), 3.97-3.86 (m, 2H), 3.71 (d, J = 11.8 Hz, 1H), 3.61 (s, 1H), 3.24 (dd, J = 9.3, 3.7 Hz, 1H), 2.73-2.62 (m, 1H), 2.27-1.95 (m, 3H), 1.93-1.48 (m, 5H), 1.45-1.04 (m, 5H). | 515.3 | *** |
| 27 | 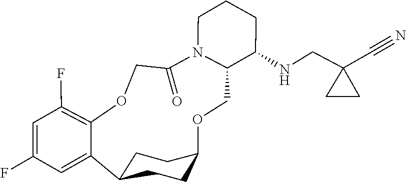 [SMILES: N#CC1(CN[C@H]2CCCN3C(=O)COc4c(F)cc(F)cc4[C@H]4CC[C@H](CC4)OC[C@@H]23)CC1] | 1- ((((2¹S,2⁴S,5²R,5³S)- 13,15-difluoro-6-oxo- 3,8-dioxa-5(2,1)- piperidina-1(1,2)- benzena-2(1,4)- cyclohexanacyclo- octaphane-5³- yl)amino)methyl) cyclopropane-1- carbonitrile | 1H NMR (300 MHz, Acetonitrile-d3) δ 6.80- 6.94 (m, 1H), 6.70- 6.80 (m, 1H), 5.39 (dd, J = 12.2, 3.4 Hz, 1H), 5.17-5.04 (m, 1H), 4.13 (dd, J = 12.2, 1.9 Hz, 1H), 3.76-3.89 (m, 2H), 3.70 (s, 1H), 3.35- 3.61 (m, 2H), 2.77- 2.91 (m, 2H), 2.71- 2.77 (m, 1H), 2.47- 2.70 (m, 2H), 2.08- 2.16 (m, 1H), 2.00- 2.08 (m, 1H), 1.68- 1.85 (m, 3H), 1.29- 1.65 (m, 7H), 1.17- 1.22 (m, 2H), 1.10- 1.17 (m, 1H), 0.88- 1.02 (m, 2H). 19F NMR (282 MHz, Acetonitrile- d3) δ −119.23, −127.68. | 460.25 | *** |
| 28 | 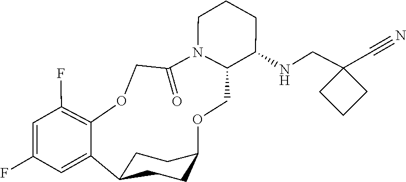 [SMILES: N#CC1(CN[C@H]2CCCN3C(=O)COc4c(F)cc(F)cc4[C@H]4CC[C@H](CC4)OC[C@@H]23)CCC1] | 1- ((((2¹S,2⁴S,5²R,5³S)- 13,15-difluoro-6-oxo- 3,8-dioxa-5(2,1)- piperidina-1(1,2)- benzena-2(1,4)- cyclohexanacyclo- octaphane-5³- yl)amino)methyl) cyclobutane-1- carbonitrile | ¹H NMR (400 MHz, CD₃OD) δ 6.86 (ddd, J = 11.5, 8.3, 3.1 Hz, 1H), 6.74 (ddd, J = 9.1, 3.0, 1.9 Hz, 1H), 5.47 (dd, J = 12.4, 3.2 Hz, 1H), 5.26-5.16 (m, 1H), 4.14 (dd, J = 12.4, 1.8 Hz, 1H), 3.95-3.82 (m, 2H), 3.71 (s, 1H), 3.58- 3.44 (m, 2H), 3.03 (s, 2H), 2.94-2.83 (m, 1H), 2.77-2.55 (m, 2H), 2.52-2.39 (m, 2H), 2.30-2.00 (m, 6H), 1.94-1.78 (m, 3H), 1.72-1.27 (m, 6H) | 474.2 | *** |
| 29 | 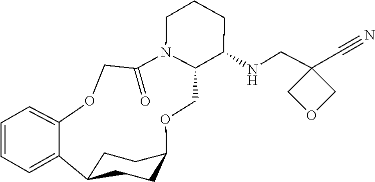 [SMILES: N#CC1(CN[C@H]2CCCN3C(=O)COc4ccccc4[C@H]4CC[C@H](CC4)OC[C@@H]23)COC1] | 3- ((((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)methyl) oxetane-3-carbonitrile | ¹H NMR (400 MHz, CD₃OD) δ 7.14 (td, J = 7.8, 1.7 Hz, 1H), 7.06 (dd, J = 7.4, 1.6 Hz, 1H), 6.89-6.81 (m, 2H), 5.29 (d, J = 10.6 Hz, 1H), 5.25-5.17 (m, 1H), 4.86 (dd, J = 6.4, 3.0 Hz, 2H), 4.60 (dd, J = 8.3, 6.4 Hz, 2H), 4.10 (d, J = 10.6 Hz, 1H), 3.93 (dd, J = 10.9, 9.6 Hz, 1H), 3.79 (d, J = 15.0 Hz, 1H), 3.68 (s, 1H), 3.54-3.44 (m, 2H), 2.97-2.87 (m, 1H), 2.73 (qd, J = 12.9, 4.4 Hz, 1H), 2.61-2.50 (m, 1H), 2.31 (ddd, J = 26.0, 13.1, 4.2 Hz, 1H), 2.16 (d, J = 14.0 Hz, 1H), 1.84 (ddd, J = 19.5, 15.2, 4.8 Hz, 3H), 1.71- 1.20 (m, 8H) | 440.3 | *** |
| 30 | 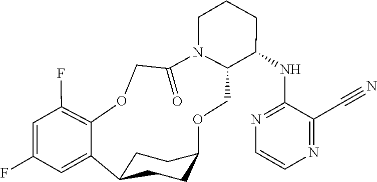 [SMILES: N#Cc1nccnc1N[C@H]1CCCN2C(=O)COc3c(F)cc(F)cc3[C@H]3CC[C@H](CC3)OC[C@@H]12] | 3- (((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)pyrazine-2- carbonitrile | ¹H NMR (400 MHz, CD₃OD) δ 8.34 (d, J = 2.4 Hz, 1H), 7.92 (d, J = 2.4 Hz, 1H), 6.86 (ddd, J = 11.5, 8.3, 3.1 Hz, 1H), 6.77-6.70 (m, 1H), 5.56-5.40 (m, 2H), 4.43-4.32 (m, 1H), 4.15 (dd, J = 12.4, 1.7 Hz, 1H), 4.03-3.92 (m, 2H), 3.68 (s, 1H), 3.62-3.49 (m, 1H), 3.36 (dd, J = 9.0, 3.6 Hz, 1H), 2.80-2.53 (m, 2H), 2.20 (d, J = 14.0 Hz, 1H), 2.14-1.68 (m, 7H), 1.55-1.26 (m, 4H). | 484.7 | *** |
| 31 | 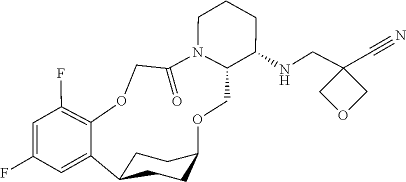 [SMILES: N#CC1(CN[C@H]2CCCN3C(=O)COc4c(F)cc(F)cc4[C@H]4CC[C@H](CC4)OC[C@@H]23)COC1] | 3- ((((2¹S,2⁴S,5²R,5³S)- 13,15-difluoro-6-oxo- 3,8-dioxa-5(2,1)- piperidina-1(1,2)- benzena-2(1,4)- cyclohexanacyclo- octaphane-5³- yl)amino)methyl) oxetane-3-carbonitrile | ¹H NMR (400 MHz, CD₃OD) δ 6.85 (ddd, J = 11.5, 8.3, 3.1 Hz, 1H), 6.78-6.69 (m, 1H), 5.46 (dd, J = 12.4, 3.2 Hz, 1H), 5.26-5.17 (m, 1H), 4.87-4.83 (m, 2H), 4.59 (dd, J = 7.9, 6.5 Hz, 2H), 4.14 (dd, J = 12.4, 1.7 Hz, 1H), 3.97-3.81 (m, 2H), 3.70 (s, 1H), 3.55-3.42 (m, 2H), 3.29 (d, J = 2.1 Hz, 2H), 2.96-2.85 (m, 1H), 2.73-2.56 (m, 2H), 2.21-2.02 (m, 2H), 1.94-1.79 (m, 3H), 1.70-1.28 (m, 7H) | 476.3 | *** |
| 32 | 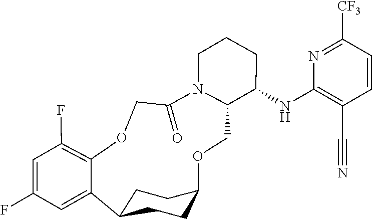 [SMILES: N#Cc1ccc(C(F)(F)F)nc1N[C@H]1CCCN2C(=O)COc3c(F)cc(F)cc3[C@H]3CC[C@H](CC3)OC[C@@H]12] | 2- (((2¹S,2⁴S,5²R,5³S)- 13,15-difluoro-6-oxo- 3,8-dioxa-5(2,1)- piperidina-1(1,2)- benzena-2(1,4)- cyclohexanacyclo- octaphane-5³- yl)amino)- 6- (trifluoromethyl) nicotinonitrile | 1H NMR (400 MHz, DMSO) δ 8.25 (d, J = 7.8 Hz, 1H), 7.55 (d, J = 7.4 Hz, 1H), 7.22-7.10 (m, 2H), 6.88 (d, J = 9.2 Hz, 1H), 5.44 (dd, J = 12.2, 3.0 Hz, 1H), 5.31- 5.19 (m, 1H), 4.27- 4.13 (m, 1H), 3.98 (d, J = 12.0 Hz, 1H), 3.89- 3.73 (m, 2H), 3.62 (s, 1H), 3.36-3.22 (m, 1H), 2.64-2.56 (m, 2H), 2.17-1.88 (m, 3H), 1.88-1.68 (m, 2H), 1.68-1.50 (m, 2H), 1.46-1.16 (m, 4H). | 551.7 | *** |
| 33 | 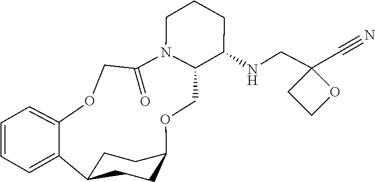 [SMILES: N#CC1(CN[C@H]2CCCN3C(=O)COc4ccccc4[C@H]4CC[C@H](CC4)OC[C@@H]23)CCO1] | 2- ((((2¹S,2⁴S,5²R,5³S)- 6-oxo-3,8-dioxa- 5(2,1)-piperidina- 1(1,2)-benzena- 2(1,4)- cyclohexanacyclo- octaphane-5³- yl)amino)methyl) oxetane-2-carbonitrile | ¹H NMR (400 MHz, CD₃OD) δ 7.14 (td, J = 7.8, 1.7 Hz, 1H), 7.06 (dd, J = 7.4, 1.7 Hz, 1H), 6.90-6.79 (m, 2H), 5.29 (d, J = 10.6 Hz, 1H), 5.19 (s, 1H), 4.76 (dt, J = 14.2, 7.1 Hz, 1H), 4.62-4.49 (m, 1H), 4.10 (d, J = 10.6 Hz, 1H), 3.97-3.88 (m, 1H), 3.79 (d, J = 12.8 Hz, 1H), 3.68 (s, 1H), 3.59-3.44 (m, 2H), 3.25-3.09 (m, 2H), 3.07-2.90 (m, 3H), 2.78-2.63 (m, 1H), 2.61-2.50 (m, 1H), 2.34-2.25 (m, 1H), 2.17 (d, J = 12.6 Hz, 1H), 1.85 (dd, J = 28.3, 14.7 Hz, 3H), 1.65- 1.32 (m, 7H) | 440.8 | *** |

pEC₅₀ Bins
*** = 0.01-1 nM (pEC₅₀ 11-9)
** = 1-100 nM (pEC₅₀ 7-9)
* = 100-1000 nM (pEC₅₀ 5-7)

### INCORPORATION BY REFERENCE

All of the U.S. patents and U.S. and PCI patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

While there have been shown and described what are at present considered the preferred embodiments of the invention, those skilled in the art may make various changes and modifications which remain within the scope of the invention defined by the appended claims.

What is claimed is:

1. A compound according to Formula (A): 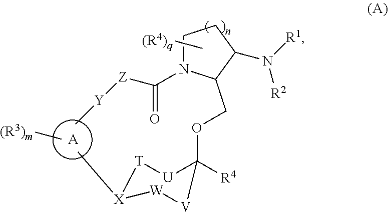
or deuterated derivative, or a pharmaceutically acceptable salt or prodrug thereof;
wherein:
A is selected from the group consisting of phenyl, pyridinyl, pyridazinyl, pyrimidinyl, and pyrazinyl;
n is 0, 1, 2, or 3;
R¹ is (C₁-C₆)alkyl, (C₃-C₈)cycloalkyl, phenyl, 4- to 7-membered heterocycloalkyl, or 5- to 6-membered heteroaryl; wherein each (C₁-C₆)alkyl is optionally substituted with one or more R^{1a}, C(O)NR^{1b}R^{1c}, halo, cyano, hydroxy, or (C₁-C₆)alkoxy; and each (C₃-C₈)cycloalkyl, phenyl, 4- to 7-membered heterocycloalkyl, and 5- to 6-membered heteroaryl, is optionally substituted with one or more of halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl;
R^{1a} is (C₃-C₈)cycloalkyl, phenyl, 4- to 7-membered heterocycloalkyl, or 5- to 6-membered heteroaryl, each of which is optionally substituted with one or more of halo, cyano, hydroxy, (C₁-C₆)alkyl, (C₁-C₆)haloalkyl, (C₁-C₆)hydroxyalkyl, (C₁-C₆)alkoxy(C₁-C₆)alkyl, or (C₁-C₆)alkoxy;
R^{1b} and R^{1c} are independently H or (C₁-C₆)alkyl, wherein each (C₁-C₆)alkyl is optionally substituted with one or more halo, cyano, hydroxy, or (C₁-C₆)alkoxy; or R^{1a} and R^{1b} taken together with the nitrogen to which they are attached form a 3- to 7-membered heterocyclic ring;
R² is H;
R³ is independently selected from the group consisting of halo, cyano, (C₁-C₆)alkyl, and (C₁-C₆)haloalkyl;
m is 0, 1, 2, 3, or 4;
q is 0, 1, or 2;
R⁴ is, in each instance, independently selected from the group consisting of hydrogen atom (H), halo, (C₁-C₆)alkyl, and (C₁-C₆)haloalkyl;
X is CH;
Y is O, or absent;
Z is O, or (CR⁷R⁸)ₚ;
p is 1, 2, 3, or 4;
R⁷ and R⁸ are independently for each occurrence H, (C₁-C₃)alkyl, or (C₁-C₃)haloalkyl; or R⁷ and R⁸ taken together with the carbon to which they are attached form a 3- to 6-membered cycloalkyl;
T is CR⁹R¹⁰ or absent
U is CR¹¹R¹²;
V is CR¹³R¹⁴;
W is CR¹⁵R¹⁶ or absent, provided that only one of T or W is absent; and
R⁹, R¹⁰, R¹¹, R¹², R¹³, R¹⁴, R¹⁵, and R¹⁶ are each independently H or fluoro.

2. The compound of claim 1, having the structure of Formula Ia: 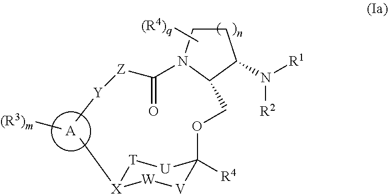
or deuterated derivative, or a pharmaceutically acceptable salt or prodrug thereof.

3. The compound of claim 1, having the structure of Formula Ia: 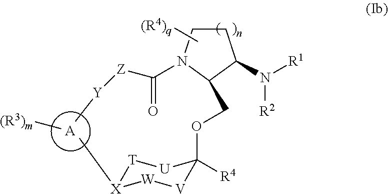
or deuterated derivative, or a pharmaceutically acceptable salt or prodrug thereof.

4. The compound of any one of claims 1-3, wherein n is 1.

5. The compound of any one of claims 1-3, wherein n is 2.

6. The compound of any one of claims 1-5, wherein A is phenyl.

7. The compound of any one of claims 1-6, wherein m is 0.

8. The compound of any one of claims 1-6, wherein in is 1.

9. The compound of any one of claims 1-6, wherein m is 2.

10. The compound of any one of claims 1-9, wherein R³ is halo.

11. The compound of claim 10, wherein R³ is fluoro.

12. A compound according to Formula (I): 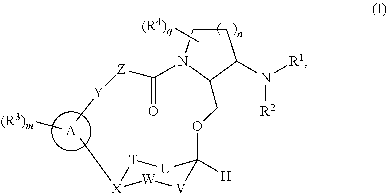
or a pharmaceutically acceptable salt thereof;
wherein:
A is selected from the group consisting of phenyl, pyridinyl, pyridazinyl, pyrimidinyl, and pyrazinyl;
n is 0, 1, 2, or 3;
R¹ is (C₁-C₆)alkyl, (C₃-C₈)cycloalkyl, phenyl, 4- to 7-membered heterocycloalkyl, or 5- to 6-membered heteroaryl; wherein each (C₁-C₆)alkyl is optionally substituted with one or more R^{1a}, C(O)NR^{1b}R^{1c}, halo, cyano, hydroxy, or (C₁-C₆)alkoxy; and each (C₃-C₈)cycloalkyl, phenyl, 4- to 7-membered heterocycloalkyl, and 5- to 6-membered heteroaryl, is optionally substituted with one or more of halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl;
R^{1a} is (C₃-C₈)cycloalkyl, phenyl, 4- to 7-membered heterocycloalkyl, or 5- to 6-membered heteroaryl, each of which is optionally substituted with one or more of halo, cyano, hydroxy, (C₁-C₆)alkyl, (C₁-C₆)haloalkyl, (C₁-C₆)hydroxyalkyl, (C₁-C₆)alkoxy(C₁-C₆)alkyl, or (C₁-C₆)alkoxy;
R^{1b} and R^{1c} are independently H or (C₁-C₆)alkyl, wherein each (C₁-C₆)alkyl is optionally substituted with one or more halo, cyano, hydroxy, or (C₁-C₆)alkoxy; or R^{1a} and R^{1b} taken together with the nitrogen to which they are attached form a 3- to 7-membered heterocyclic ring;
R² is H;
R³ is independently selected from the group consisting of halo, cyano, (C₁-C₆)alkyl, and (C₁-C₆)haloalkyl;
m is 0, 1, 2, 3, or 4;
q is 0, 1, or 2;
R⁴ is halo, (C₁-C₆)alkyl, or (C₁-C₆)haloalkyl;
X is CH;
Y is O, or absent;
Z is O, or (CR⁷R⁸)ₚ;
p is 1, 2, 3, or 4;
R⁷ and R⁸ are independently for each occurrence H, (C₁-C₃)alkyl, or (C₁-C₃)haloalkyl; or
R⁷ and R⁸ taken together with the carbon to which they are attached form a 3- to 6-membered cycloalkyl;
T is CR⁹R¹⁰ or absent
U is CR¹¹R¹²;
V is CR¹³R¹⁴;
W is CR¹⁵R¹⁶ or absent, provided that only one of T or W is absent; and
R⁹, R¹⁰, R¹¹, R¹², R¹³, R¹⁴, R¹⁵, and R¹⁶ are each independently H or fluoro.

13. The compound of claim 12, having the structure of Formula Ia-1: 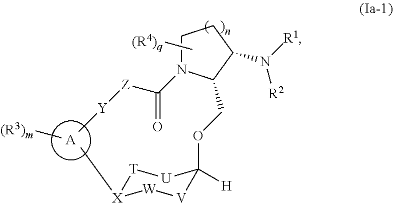
or a pharmaceutically acceptable salt thereof.

14. The compound of claim 1, having the structure of Formula Ib-1: 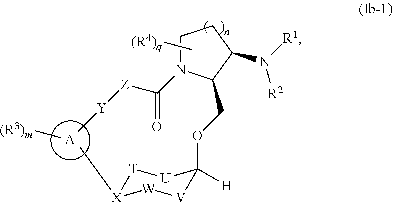
or a pharmaceutically acceptable salt thereof.

15. The compound of any one of claims 1-3 and 12-14, wherein n is 1.

16. The compound of any one of claims 1-3 and 12-14, wherein n is 2.

17. The compound of any one of claims 1-3 and 12-16, wherein A is phenyl.

18. The compound of any one of claims 1-6 and 12-17, wherein m is 0.

19. The compound of any one of claims 1-6 and 12-17, wherein m is 1.

20. The compound of any one of claims 1-6 and 12-17, wherein m is 2.

21. The compound of any one of claims 1-3 and 12-20, wherein R³ is halo.

22. The compound of claim 21, wherein R³ is fluoro.

23. The compound of any one of claims 1-5 and 12-22, wherein A is

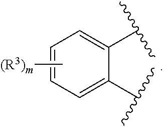

24. The compound of claim 23, wherein A is

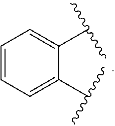 [SMILES: *c1ccccc1*]

25. The compound of claim 23, wherein A is

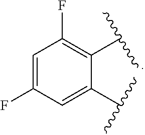 [SMILES: *c1cc(F)cc(F)c1*]

26. The compound of any one of claims 1-3 and 12-25, wherein R¹ is (C₁-C₆)alkyl optionally substituted with one or more of fluoro, cyano, hydroxy, R^{1a}, or C(O)NR^{1b}R^{1c}.

27. The compound of claim 26, wherein R¹ is methyl optionally substituted with one or more fluoro, cyano, hydroxy, R^{1a}, or C(O)NR^{1b}R^{1c}.

28. The compound of any one of claims 1-3 and 12-27, wherein R^{1a} is (C₃-C₈)cycloalkyl, phenyl, 4- to 5-membered heterocycloalkyl, or 5- to 6-membered heteroaryl, each of which is optionally substituted with one or more of fluoro, cyano, (C₁-C₃)alkyl, (C₁-C₃)fluoroalkyl, or (C₁-C₃)alkoxy.

29. The compound of any one of claims 1-3 and 12-27, wherein R^{1a} is cyclopropyl, phenyl, 4- to 5-membered heterocycloalkyl, or 5- to 6-membered heteroaryl, each of which is optionally substituted with one or more of fluoro, cyano, cyclopropyl, methyl, or trifluoromethyl.

30. The compound of any one of claims 1-3 and 12-27, wherein R^{1a} is cyclopropyl, cyclobutyl, phenyl, oxetanyl, tertrahydrofuranyl, or tetrahydropyranyl, each of which is optionally substituted with one or more of fluoro, cyano, cyclopropyl, methyl, or trifluoromethyl.

31. The compound of any one of claims 1-3 and 12-27, wherein R¹ is (C₁-C₆)alkyl substituted with C(O)NR^{1b}R^{1c}.

32. The compound of any one of claims 1-3, 12-27, and 31, wherein R^{1b} and R^{1c} are each hydrogen.

33. The compound of any one of claims 12-25, wherein R¹ is

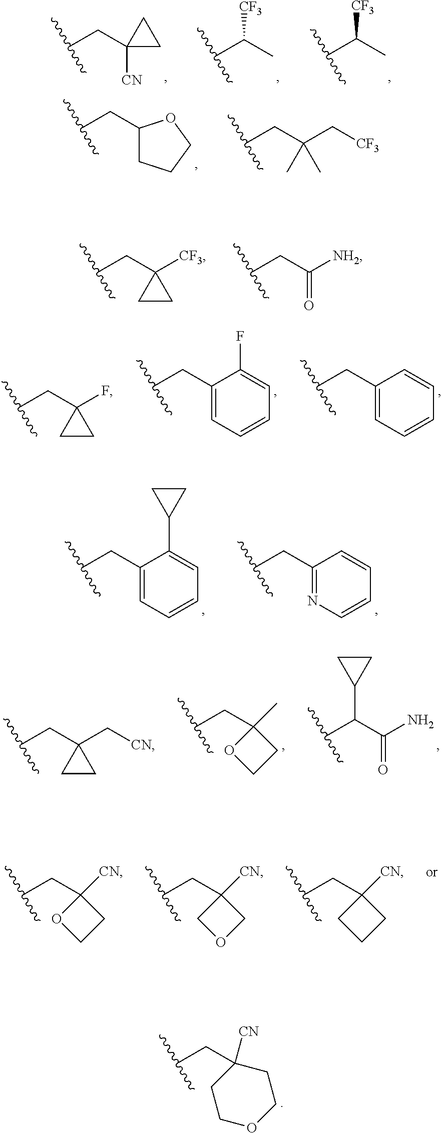

34. The compound of any one of claims 12-25, wherein R¹ is

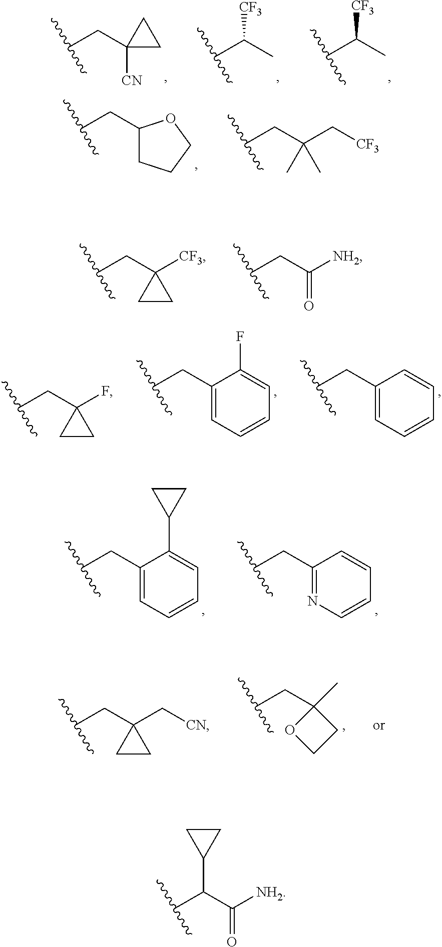

35. The compound of any one of claims 1-3 and 12-25, wherein R¹ is phenyl, 2-pyridyl, 3-pyridyl, 4-pyridyl, pyridazinyl, pyrazinyl, or diazoyl, each of which is optionally substituted with one or more of halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl.

36. The compound of any one of claims 1-3 and 12-25, wherein R¹ is phenyl, 2-pyridyl, 3-pyridyl, 4-pyridyl, or diazoyl, each of which is optionally substituted with one or more of halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl.

37. The compound of any one of claims 1-3 and 12-25, wherein R¹ is phenyl, 2-pyridyl, 3-pyridyl, 4-pyridyl, pyridazinyl, pyrazinyl, or diazoyl, each of which is optionally substituted with one or more of fluoro, cyano, hydroxy, (C₁-C₃)alkyl, (C₁-C₃)fluoroalkyl, or (C₁-C₃)alkoxy.

38. The compound of any one of claims 1-3 and 12-25, wherein R¹ is phenyl, 2-pyridyl, 3-pyridyl, 4-pyridyl, or diazoyl, each of which is optionally substituted with one or more of fluoro, cyano, hydroxy, (C₁-C₃)alkyl, (C₁-C₃)fluoroalkyl, (C₁-C₃)alkoxy or 4- to 7-membered heterocycloalkyl.

39. The compound of any one of claims 1-3 and 12-25, wherein R¹ is phenyl, 2-pyridyl, 3-pyridyl, 4-pyridyl, or diazoyl, each of which is optionally substituted with one or more of fluoro, cyano, methyl, trifluoromethyl, methoxy or oxetane.

40. The compound of any one of claims 1-3 and 12-25, wherein R¹ is

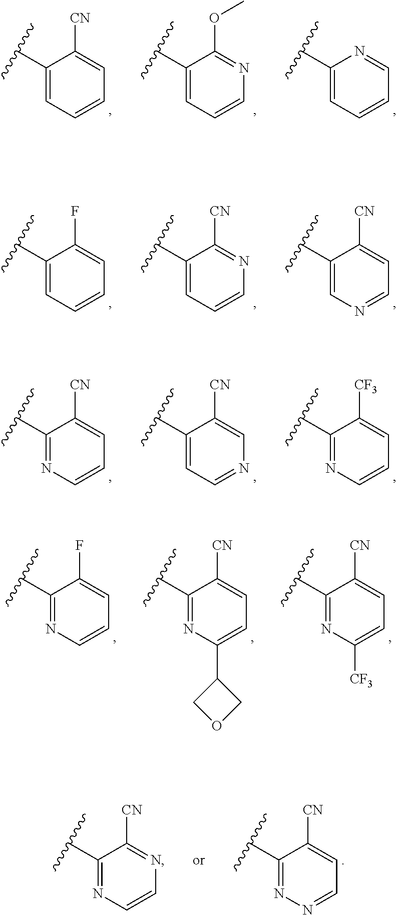

41. The compound of any one of claims 1-3 and 12-25, wherein R¹ is:

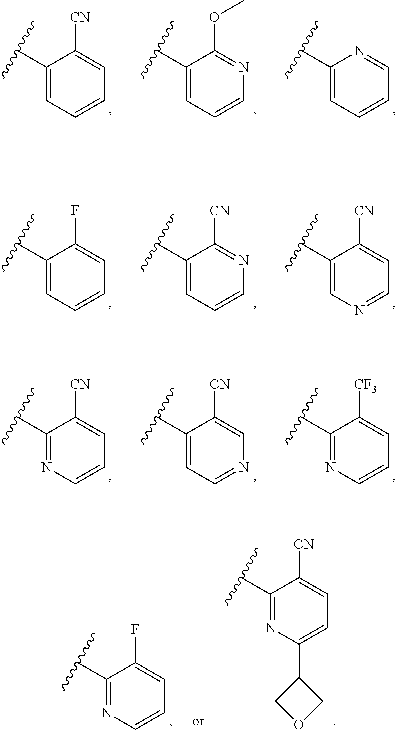

42. The compound of any one of claims 1-3 and 12-25, wherein R¹ is (C₃-C₅)cycloalkyl or 4- to 6-membered heterocycloalkyl, each of which is optionally substituted with one or more of halo, cyano, hydroxy, (C₁-C₆)alkyl, (C₁-C₆)haloalkyl, (C₁-C₆)hydroxyalkyl, (C₁-C₆)alkoxyalkyl, (C₁-C₆)alkoxy, (C₃-C₈)cycloalkyl, or 4- to 7-membered heterocycloalkyl.

43. The compound of claim 42, wherein R¹ is:

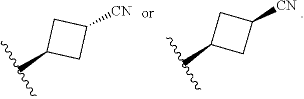 [SMILES: *[C@H]1C[C@H](C#N)C1 ; *[C@H]1C[C@@H](C#N)C1]

44. The compound of claim 42, wherein R¹ is:

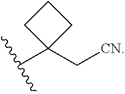 [SMILES: *C1(CC#N)CCC1]

45. The compound of claim 42, wherein R¹ is:

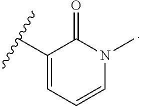 [SMILES: *c1cccn(C)c1=O]

46. The compound of any one of claims 1-3 and 12-45, wherein R¹ is substituted with at least one of fluoro or cyano.

47. The compound of any one of claims 1-3 and 12-46, wherein Y is O.

48. The compound of any one of claims 1-47, wherein R⁷ and R⁸ are each CH₃; or R⁷ and R⁸ taken together with the atom to which they are attached form a cyclopropyl.

49. The compound of any one of claims 1-47, wherein R⁷ and R⁸ are each H.

50. The compound of any one of claims 1-49, wherein p is 1.

51. The compound of any one of claims 1-50, wherein R⁹, R¹⁰, R¹¹, R¹², R¹³, R¹⁴, R¹⁵ and R¹⁶ are each H.

52. A compound selected from the following table:

| Cmpd # | Structure | IUPAC |
|---|---|---|
| 1 | 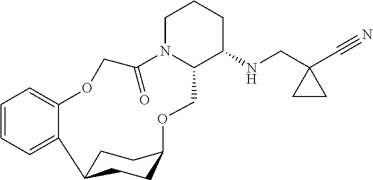 [SMILES: N#CC1(CN[C@H]2CCCN3C(=O)COc4ccccc4[C@H]4CC[C@H](CC4)OC[C@@H]23)CC1] | 1-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)cyclopropane-1-carbonitrile |
| 2 | 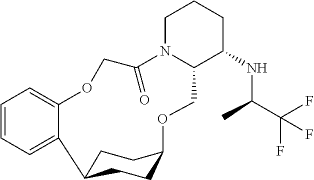 [SMILES: C[C@@H](N[C@H]1CCCN2C(=O)COc3ccccc3[C@H]3CC[C@H](CC3)OC[C@@H]12)C(F)(F)F] | (2¹S,2⁴S,5²R,5³S)-5³-(((R)-1,1,1-trifluoropropan-2- yl)amino)-3,8-dioxa-5(2,1)-piperidina-1(1,2)-benzena- 2(1,4)-cyclohexanacyclooctaphan-6-one |
| 3 | 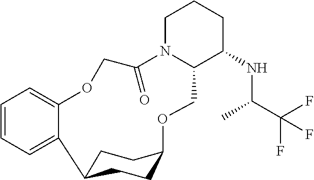 [SMILES: C[C@H](N[C@H]1CCCN2C(=O)COc3ccccc3[C@H]3CC[C@H](CC3)OC[C@@H]12)C(F)(F)F] | (2¹S,2⁴S,5²R,5³S)-5³-(((S)-1,1,1-trifluoropropan-2- yl)amino)-3,8-dioxa-5(2,1)-piperidina-1(1,2)-benzena- 2(1,4)-cyclohexanacyclooctaphan-6-one |
| 4 | 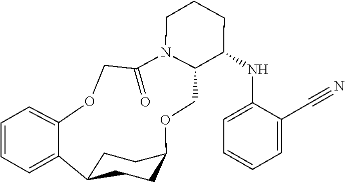 [SMILES: N#Cc1ccccc1N[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)benzonitrile |
| 5 | 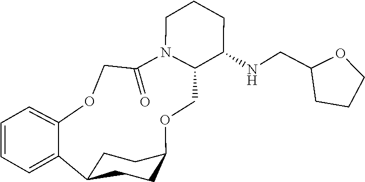 [SMILES: O=C1COc2ccccc2[C@H]2CC[C@H](CC2)OC[C@H]2[C@@H](NCC3CCCO3)CCCN12] | (2¹S,2⁴S,5²R,5³S)-5³-(((tetrahydrofuran-2- yl)methyl)amino)-3,8-dioxa-5(2,1)-piperidina-1(1,2)- benzena-2(1,4)-cyclohexanacyclooctaphan-6-one |
| 6 | 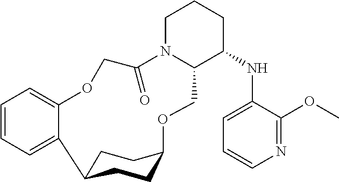 | (2¹S,2⁴S,5²R,5³S)-5³-((2-methoxypyridin-3-yl)amino)- 3,8-dioxa-5(2,1)-piperidina-1(1,2)-benzena-2(1,4)- cyclohexanacyclooctaphan-6-one |
| 7 | 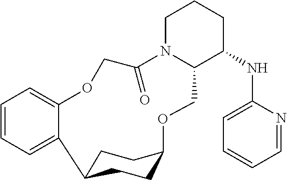 [SMILES: O=C1COc2ccccc2[C@H]2CC[C@H](CC2)OC[C@H]2[C@@H](Nc3ccccn3)CCCN12] | (2¹S,2⁴S,5²R,5³S)-5³-(pyridin-2-ylamino)-3,8-dioxa- 5(2,1)-piperidina-1(1,2)-benzena-2(1,4)- cyclohexanacyclooctaphan-6-one |
| 8 | 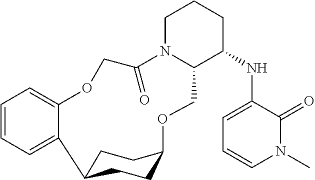 [SMILES: Cn1cccc(N[C@H]2CCCN3C(=O)COc4ccccc4[C@H]4CC[C@H](CC4)OC[C@@H]23)c1=O] | (2¹S,2⁴S,5²R,5³S)-5³-((1-methyl-2-oxo-1,2- dihydropyridin-3-yl)amino)-3,8-dioxa-5(2,1)- piperidina-1(1,2)-benzena-2(1,4)- cyclohexanacyclooctaphan-6-one |
| 9 | 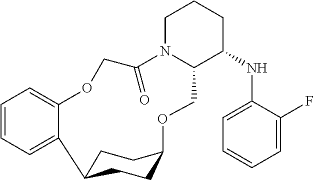 [SMILES: O=C1COc2ccccc2[C@H]2CC[C@H](CC2)OC[C@H]2[C@@H](Nc3ccccc3F)CCCN12] | (2¹S,2⁴S,5²R,5³S)-5³-((2-fluorophenyl)amino)-3,8- dioxa-5(2,1)-piperidina-1(1,2)-benzena-2(1,4)- cyclohexanacyclooctaphan-6-one |
| 10 | 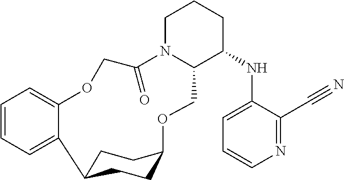 [SMILES: N#Cc1ncccc1N[C@H]1CCCN2C(=O)COc3ccccc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)picolinonitrile |
| 11 | 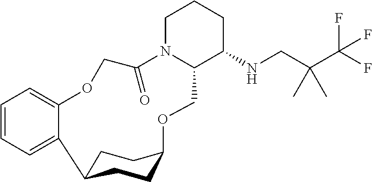 | (2¹S,2⁴S,5²R,5³S)-5³-((3,3,3-trifluoro-2,2- dimethylpropyl)amino)-3,8-dioxa-5(2,1)-piperidina- 1(1,2)-benzena-2(1,4)-cyclohexanacyclooctaphan-6-one |
| 12 | 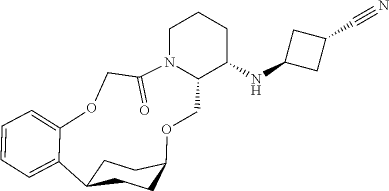 [SMILES: N#C[C@H]1C[C@H](N[C@H]2CCCN3C(=O)COc4ccccc4[C@H]4CC[C@H](CC4)OC[C@@H]23)C1] | (1S,3r)-3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)cyclobutane-1- carbonitrile |
| 13 | 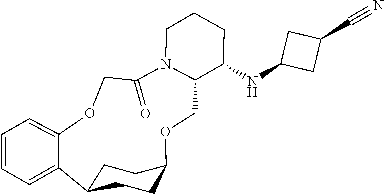 [SMILES: N#C[C@H]1C[C@@H](N[C@H]2CCCN3C(=O)COc4ccccc4[C@H]4CC[C@H](CC4)OC[C@@H]23)C1] | (1R,3s)-3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)cyclobutane-1- carbonitrile |
| 14 | 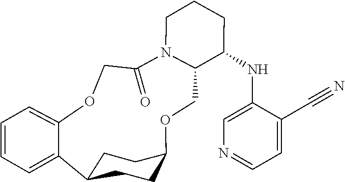 [SMILES: N#Cc1ccncc1N[C@H]1CCCN2C(=O)COc3ccccc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)isonicotinonitrile |
| 15 | 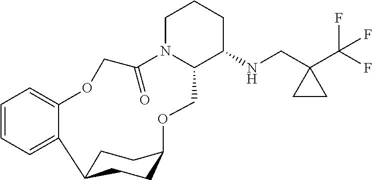 [SMILES: O=C1COc2ccccc2[C@H]2CC[C@H](CC2)OC[C@H]2[C@@H](NCC3(C(F)(F)F)CC3)CCCN12] | (2¹S,2⁴S,5²R,5³S)-5³-(((1- (trifluoromethyl)cyclopropyl)methyl)amino)-3,8-dioxa- 5(2,1)-piperidina-1(1,2)-benzena-2(1,4)- cyclohexanacyclooctaphan-6-one |
| 16 | 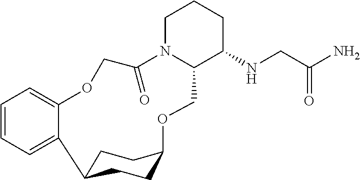 [SMILES: NC(=O)CN[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)acetamide |
| 17 | 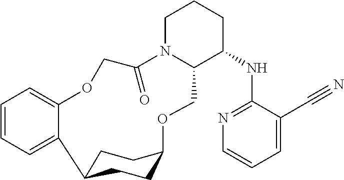 [SMILES: N#Cc1cccnc1N[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)nicotinonitrile |
| 18 | 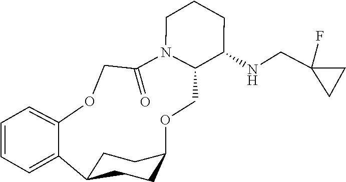 [SMILES: O=C1COc2ccccc2[C@H]2CC[C@H](CC2)OC[C@H]2[C@@H](NCC3(F)CC3)CCCN12] | (2¹S,2⁴S,5²R,5³S)-5³-(((1- fluorocyclopropyl)methyl)amino)-3,8-dioxa-5(2,1)- piperidina-1(1,2)-benzena-2(1,4)- cyclohexanacyclooctaphan-6-one |
| 19 | 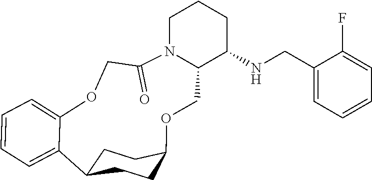 [SMILES: O=C1COc2ccccc2[C@H]2CC[C@H](CC2)OC[C@H]2[C@@H](NCc3ccccc3F)CCCN12] | (2¹S,2⁴S,5²R,5³S)-5³-((2-fluorobenzyl)amino)-3,8- dioxa-5(2,1)-piperidina-1(1,2)-benzena-2(1,4)- cyclohexanacyclooctaphan-6-one |
| 20 | 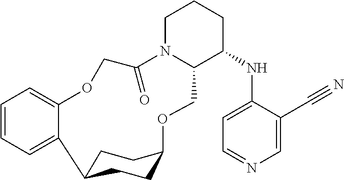 [SMILES: N#Cc1cnccc1N[C@H]1CCCN2C(=O)COc3ccccc3[C@H]3CC[C@H](CC3)OC[C@@H]12] | 4-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)nicotinonitrile |
| 21 | 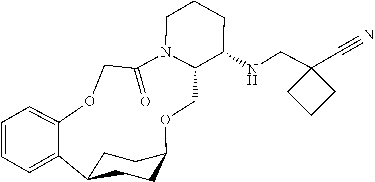 [SMILES: N#CC1(CN[C@H]2CCCN3C(=O)COc4ccccc4[C@H]4CC[C@H](CC4)OC[C@@H]23)CCC1] | 1-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)cyclobutane-1-carbonitrile |
| 22 | 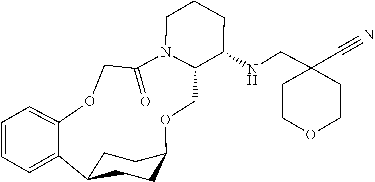 [SMILES: N#CC1(CN[C@H]2CCCN3C(=O)COc4ccccc4[C@H]4CC[C@H](CC4)OC[C@@H]23)CCOCC1] | 4-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)tetrahydro-2H-pyran-4-carbonitrile |
| 23 | 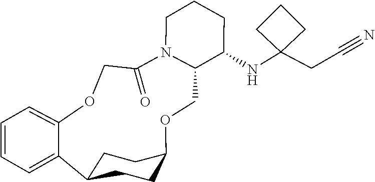 [SMILES: N#CCC1(N[C@H]2CCCN3C(=O)COc4ccccc4[C@H]4CC[C@H](CC4)OC[C@@H]23)CCC1] | 2-(1-(((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)cyclobutyl)acetonitrile |
| 24 | 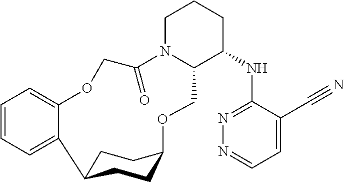 | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)pyridazine-4- carbonitrile |
| 25 | 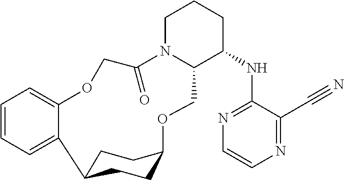 [SMILES: N#Cc1nccnc1N[C@H]1CCCN2C(=O)COc3ccccc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)pyrazine-2- carbonitrile |
| 26 | 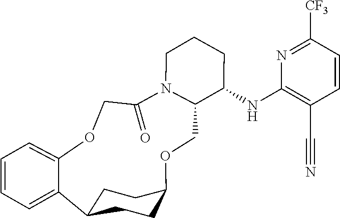 [SMILES: N#Cc1ccc(C(F)(F)F)nc1N[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)-6- (trifluoromethyl)nicotinonitrile |
| 27 | 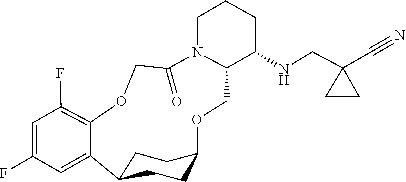 [SMILES: N#CC1(CN[C@H]2CCCN3C(=O)COc4c(F)cc(F)cc4[C@H]4CC[C@H](CC4)OC[C@@H]23)CC1] | 1-((((2¹S,2⁴S,5²R,5³S)-1³,1⁵-difluoro-6-oxo-3,8-dioxa- 5(2,1)-piperidina-1(1,2)-benzena-2(1,4)- cyclohexanacyclooctaphane-5³- yl)amino)methyl)cyclopropane-1-carbonitrile |
| 28 | 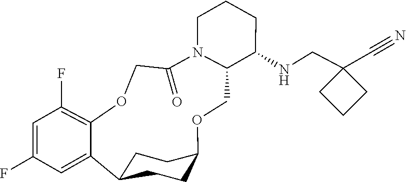 [SMILES: N#CC1(CN[C@H]2CCCN3C(=O)COc4c(F)cc(F)cc4[C@H]4CC[C@H](CC4)OC[C@@H]23)CCC1] | 1-((((2¹S,2⁴S,5²R,5³S)-1³,1⁵-difluoro-6-oxo-3,8-dioxa- 5(2,1)-piperidina-1(1,2)-benzena-2(1,4)- cyclohexanacyclooctaphane-5³- yl)amino)methyl)cyclobutane-1-carbonitrile |
| 29 | 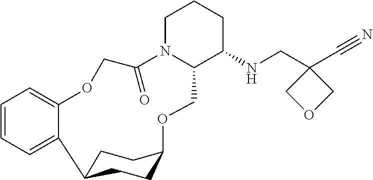 [SMILES: N#CC1(CN[C@H]2CCCN3C(=O)COc4ccccc4[C@H]4CC[C@H](CC4)OC[C@@H]23)COC1] | 3-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)oxetane-3-carbonitrile |
| 30 | 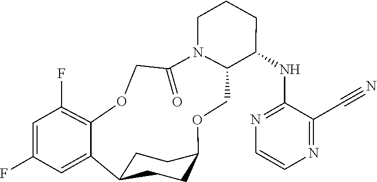 [SMILES: N#Cc1nccnc1N[C@H]1CCCN2C(=O)COc3c(F)cc(F)cc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)pyrazine-2- carbonitrile |
| 31 | 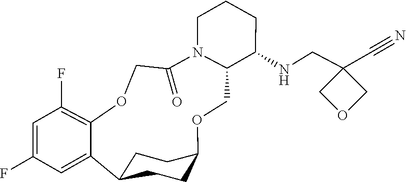 [SMILES: N#CC1(CN[C@H]2CCCN3C(=O)COc4c(F)cc(F)cc4[C@H]4CC[C@H](CC4)OC[C@@H]23)COC1] | 3-((((2¹S,2⁴S,5²R,5³S)-1³,1⁵-difluoro-6-oxo-3,8-dioxa- 5(2,1)-piperidina-1(1,2)-benzena-2(1,4)- cyclohexanacyclooctaphane-5³- yl)amino)methyl)oxetane-3-carbonitrile |
| 32 | 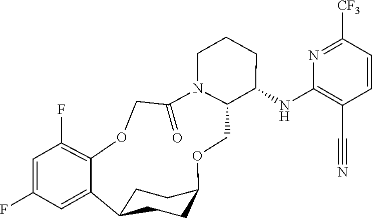 [SMILES: N#Cc1ccc(C(F)(F)F)nc1N[C@H]1CCCN2C(=O)COc3c(F)cc(F)cc3[C@H]3CC[C@H](CC3)OC[C@@H]12] | 2-(((2¹S,2⁴S,5²R,5³S)-1³,1⁵-difluoro-6-oxo-3,8-dioxa- 5(2,1)-piperidina-1(1,2)-benzena-2(1,4)- cyclohexanacyclooctaphane-5³-yl)amino)-6- (trifluoromethyl)nicotinonitrile |
| 33 | 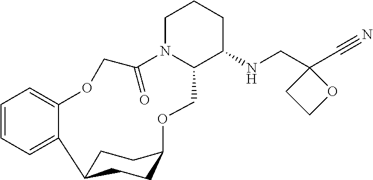 [SMILES: N#CC1(CN[C@H]2CCCN3C(=O)COc4ccccc4[C@H]4CC[C@H](CC4)OC[C@@H]23)CCO1] | 2-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)oxetane-2-carbonitrile |

or a pharmaceutically acceptable salt thereof.

53. A compound selected from the following table:

| Cmpd # | Structure | IUPAC |
|---|---|---|
| 1 | 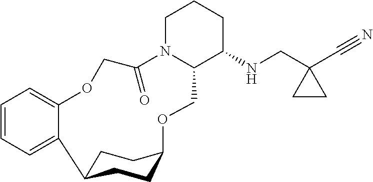 | 1-((((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)methyl)cyclopropane-1-carbonitrile |
| 2 | 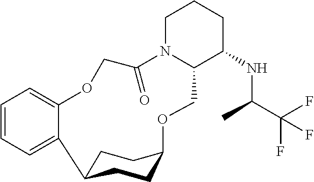 | (2¹S,2⁴S,5²R,5³S)-5³-(((R)-1,1,1-trifluoropropan-2- yl)amino)-3,8-dioxa-5(2,1)-piperidina-1(1,2)-benzena- 2(1,4)-cyclohexanacyclooctaphan-6-one |
| 3 | 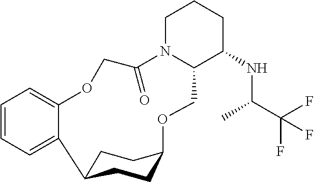 | (2¹S,2⁴S,5²R,5³S)-5³-(((S)-1,1,1-trifluoropropan-2- yl)amino)-3,8-dioxa-5(2,1)-piperidina-1(1,2)-benzena- 2(1,4)-cyclohexanacyclooctaphan-6-one |
| 4 | 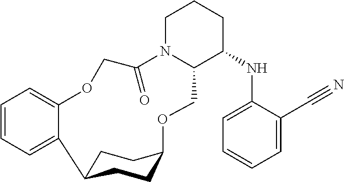 | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)benzonitrile |
| 5 | 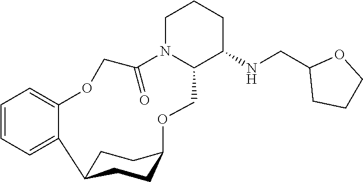 | (2¹S,2⁴S,5²R,5³S)-5³-(((tetrahydrofuran-2- yl)methyl)amino)-3,8-dioxa-5(2,1)-piperidina-1(1,2)- benzena-2(1,4)-cyclohexanacyclooctaphan-6-one |
| 6 | 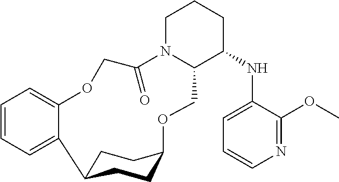 | (2¹S,2⁴S,5²R,5³S)-5³-((2-methoxypyridin-3-yl)amino)- 3,8-dioxa-5(2,1)-piperidina-1(1,2)-benzena-2(1,4)- cyclohexanacyclooctaphan-6-one |
| 7 | 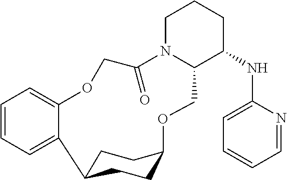 [SMILES: O=C1COc2ccccc2[C@H]2CC[C@H](CC2)OC[C@H]2[C@@H](Nc3ccccn3)CCCN12] | (2¹S,2⁴S,5²R,5³S)-5³-(pyridin-2-ylamino)-3,8-dioxa 5(2,1)-piperidina-1(1,2)-benzena-2(1,4)- cyclohexanacyclooctaphan-6-one |
| 8 | 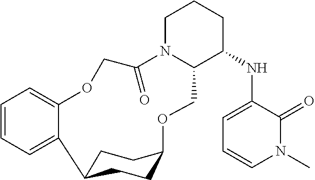 [SMILES: Cn1cccc(N[C@H]2CCCN3C(=O)COc4ccccc4[C@H]4CC[C@H](CC4)OC[C@@H]23)c1=O] | (2¹S,2⁴S,5²R,5³S)-5³-((1-methyl-2-oxo-1,2- dihydropyridin-3-yl)amino)-3,8-dioxa-5(2,1)- piperidina-1(1,2)-benzena-2(1,4)- cyclohexanacyclooctaphan-6-one |
| 9 | 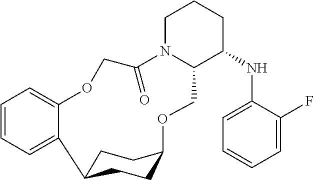 [SMILES: O=C1COc2ccccc2[C@H]2CC[C@H](CC2)OC[C@H]2[C@@H](Nc3ccccc3F)CCCN12] | (2¹S,2⁴S,5²R,5³S)-5³-((2-fluorophenyl)amino)-3,8- dioxa-5(2,1)-piperidina-1(1,2)-benzena-2(1,4)- cyclohexanacyclooctaphan-6-one |
| 10 | 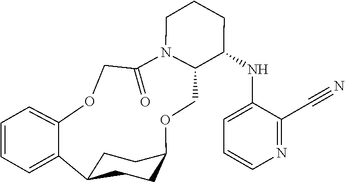 [SMILES: N#Cc1ncccc1N[C@H]1CCCN2C(=O)COc3ccccc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)picolinonitrile |
| 11 | 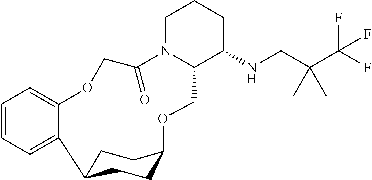 [SMILES: CC(C)(CN[C@H]1CCCN2C(=O)COc3ccccc3[C@H]3CC[C@H](CC3)OC[C@@H]12)C(F)(F)F] | (2¹S,2⁴S,5²R,5³S)-5³-((3,3,3-trifluoro-2,2- dimethylpropyl)amino)-3,8-dioxa-5(2,1)-piperidina- 1(1,2)-benzena-2(1,4)-cyclohexanacyclooctaphan-6- one |
| 12 | 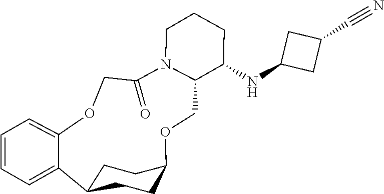 [SMILES: N#C[C@H]1C[C@H](N[C@H]2CCCN3C(=O)COc4ccccc4[C@H]4CC[C@H](CC4)OC[C@@H]23)C1] | (1S,3r)-3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)cyclobutane- 1-carbonitrile |
| 13 | 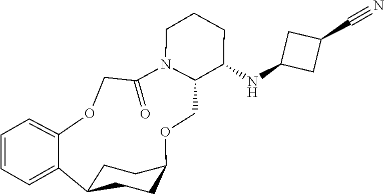 [SMILES: N#C[C@H]1C[C@@H](N[C@H]2CCCN3C(=O)COc4ccccc4[C@H]4CC[C@H](CC4)OC[C@@H]23)C1] | (1R,3s)-3-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)cyclobutane- 1-carbonitrile |
| 14 | 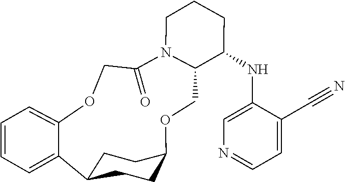 [SMILES: N#Cc1ccncc1N[C@H]1CCCN2C(=O)COc3ccccc3[C@H]3CC[C@H](CC3)OC[C@@H]12] | 3-(((2¹S,2⁴S,5²R,5³S)-6-oxo-3,8-dioxa-5(2,1)- piperidina-1(1,2)-benzena-2(1,4)- cyclohexanacyclooctaphane-5³- yl)amino)isonicotinonitrile |
| 15 | 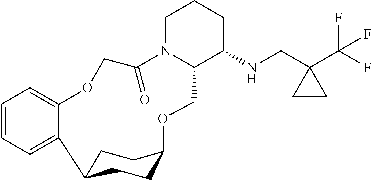 [SMILES: O=C1COc2ccccc2[C@H]2CC[C@H](CC2)OC[C@H]2[C@@H](NCC3(C(F)(F)F)CC3)CCCN12] | (2¹S,2⁴S,5²R,5³S)-5³-(((1- (trifluoromethyl)cyclopropyl)methyl)amino)-3,8-dioxa- 5(2,1)-piperidina-1(1,2)-benzena-2(1,4)- cyclohexanacyclooctaphan-6-one |
| 16 | 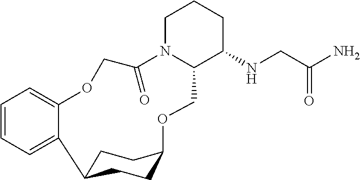 [SMILES: NC(=O)CN[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)acetamide |
| 17 | 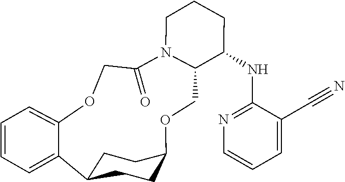 [SMILES: N#Cc1cccnc1N[C@H]1CCCN2C(=O)COc3ccccc3[C@H]3CC[C@H](CC3)OC[C@@H]12] | 2-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)nicotinonitrile |
| 18 | 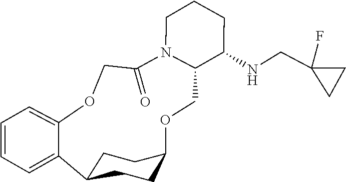 [SMILES: O=C1COc2ccccc2[C@H]2CC[C@H](CC2)OC[C@H]2[C@@H](NCC3(F)CC3)CCCN12] | (2¹S,2⁴S,5²R,5³S)-5³-(((1- fluorocyclopropyl)methyl)amino)-3,8-dioxa-5(2,1)- piperidina-1(1,2)-benzena-2(1,4)- cyclohexanacyclooctaphan-6-one |
| 19 | 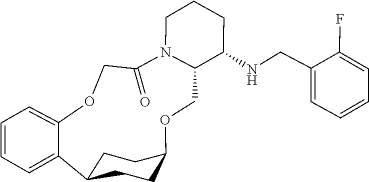 [SMILES: O=C1COc2ccccc2[C@H]2CC[C@H](CC2)OC[C@H]2[C@@H](NCc3ccccc3F)CCCN12] | (2¹S,2⁴S,5²R,5³S)-5³-((2-fluorobenzyl)amino)-3,8- dioxa-5(2,1)-piperidina-1(1,2)-benzena-2(1,4)- cyclohexanacyclooctaphan-6-one |
| 20 | 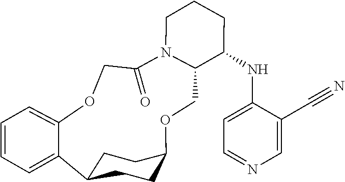 [SMILES: N#Cc1cnccc1N[C@H]1CCCN2C(=O)COc3ccccc3[C@H]3CC[C@H](CC3)OC[C@@H]12] | 4-(((2¹S,2⁴S,5²R,5³S)-6-oxo-3,8-dioxa-5(2,1)- piperidina-1(1,2)-benzena-2(1,4)- cyclohexanacyclooctaphane-5³-yl)amino)nicotinonitrile |

or a pharmaceutically acceptable salt thereof.

54. A pharmaceutical composition comprising a compound of any one of claims 1-53; and at least one pharmaceutically acceptable excipient.

55. A method of preventing or treating a disease selected from the group consisting of narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury, comprising administering to a subject in need thereof a therapeutically effective amount of a compound of any one of claims 1-53 or a pharmaceutical composition of claim 54.

56. A method of preventing or treating a disease selected from the group consisting of narcolepsy, idiopathic hypersomnia, hypersomnia, sleep apnea syndrome, narcolepsy syndrome involving narcolepsy-like symptoms, hypersomnia associated with Parkinson's disease, and hypersomnia associated with dementia with Lewy body, comprising administering to a subject in need thereof a therapeutically effective amount of a compound of any one of claims 1-53 or a pharmaceutical composition of claim 54.

57. The method of claim 56, wherein the disease is narcolepsy.

58. The method of claim 56, wherein the disease is idiopathic hypersomnia.

59. The method of claim 56, wherein the disease is hypersomnia.

60. The method of claim 56, wherein the disease is sleep apnea syndrome.

61. The method of claim 56, wherein the disease is narcolepsy syndrome involving narcolepsy-like symptoms.

62. The method of claim 56, wherein the disease is hypersomnia associated with Parkinson's disease.

63. The method of claim 56, wherein the disease is hypersomnia associated with dementia with Lewy body.

64. The method of any one of claims 55-63, wherein the compound is administered orally.

65. The method of any one of claims 55-63, wherein the compound is administered parenterally.

66. The method of any one of claims 55-63, wherein the disease is prevented.

67. The method of any one of claims 55-63, wherein the disease is treated.

68. A method of preventing or treating a disease associated with a deficiency in orexin 2 receptor signaling, comprising administering to a subject in need thereof a therapeutically effective amount of a compound of any one of claims 1-53 or a pharmaceutical composition of claim 54.

69. The method of claim 68, wherein the disease associated with a deficiency in orexin 2 receptor signaling is selected from the group consisting of narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury.

70. The method of claim 68, wherein the disease associated with a deficiency in orexin 2 receptor signaling is selected from the group consisting of narcolepsy, idiopathic hypersomnia, hypersomnia, sleep apnea syndrome, narcolepsy syndrome involving narcolepsy-like symptoms, hypersomnia associated with Parkinson's disease, and hypersomnia associated with dementia with Lewy body.

71. Use of a compound of any one of claims 1-53 or a pharmaceutical composition of claim 54 in the manufacture of a medicament for the treatment of a disease selected from selected from the group consisting of narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury.

72. A compound of any one of claims 1-53 or a pharmaceutical composition of claim 54 for use in the treatment of a disease selected from the group consisting of narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury.
